(19) United States
(12) Patent Application Publication
(10) Pub. No.: US 2021/0106703 A1
(43) Pub. Date: Apr. 15, 2021

(54) RADIOIMMUNOCONJUGATES IN COMBINATION WITH OTHER DRUGS AS TREATMENT AGAINST NHL

(71) Applicant: Nordic Nanovector ASA, Olso (NO)

(72) Inventors: Jean Pierre Pouget, Paris (FR); Alexandre Pichard, Paris (FR); Randi Syljuåsen, Oslo (NO); Gro Elise Rødland, Oslo (NO); Helen Heyerdahl, Oslo (NO); Sebastian Patzke, Oslo (NO); Jostein Dahle, Haslum (NO); Ada Repetto-Llamazares, Oslo (NO); Marion Masita Malenge, Oslo (NO)

(21) Appl. No.: 16/765,049

(22) PCT Filed: Nov. 21, 2018
(86) PCT No.: PCT/EP2018/082065

§ 371 Date: May 18, 2020

(30) Foreign Application Priority Data
Nov. 22, 2017 (EP) 17202982.9
Apr. 20, 2018 (EP) 18168529.8
Oct. 11, 2018 (EP) 18199873.3

(51) Int. Cl. A61K 51/10; A61K 31/635; C07K 16/28; A61K 39/395; A61P 35/02

(52) U.S. Cl. CPC A61K 51/1069; A61K 51/1027; A61K 31/635; C07K 16/2896; A61K 2039/507; A61K 39/39533; A61P 35/02; A61K 51/1096; C07K 16/2887

(57) ABSTRACT

The present invention relates to a combination of radioimmunoconjugates and a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, for use as a medicament. The medicament may be against Non-Hodgkin's lymphoma (NHL).

### FIELD OF THE INVENTION

The present invention relates to a combination of radioimmunoconjugates and a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, for use as a medicament. The medicament may be against Non-Hodgkin's lymphoma (NHL).

### BACKGROUND OF THE INVENTION

B-cell Non-Hodgkin Lymphoma (NHL) originates from B lymphocytes at various stages of differentiation, from precursor cells to mature stages. Currently, patients with NHL are treated by immunotherapy with the monoclonal antibody (mAb) rituximab in combination with chemotherapy.

Rituximab is a chimeric IgG1 mAb against CD20, a transmembrane protein of 33-37 kDa expressed at the surface of most malignant and normal B cells (pre-lymphocytes to pre-plasma cells). Rituximab efficacy is mediated by multiple cell death mechanisms, such as apoptosis, signal transduction pathways, antibody-dependent cell-mediated cytotoxicity (ADCC) and phagocytosis (ADCP) and complement-dependent cytotoxicity (CDC).

However, the response rate to rituximab alone is rather modest and after many cycles of treatment, some patients become refractory to this therapy. For instance, patients with recurrent follicular lymphoma (FL; a NHL type) who develop resistance to rituximab and chemotherapy and those who experience disease progression within 2 years after first-line therapy have the greatest need for new treatment approaches. The 5-year overall survival rates for patients with rituximab-refractory FL or with early disease progression are 58% and 50% compared to approximately 90% for all patients with FL.

Radioimmunotherapy (RIT), in which radiolabeled antibodies are used to combine radiation and antibody cytotoxic properties, shows significant efficacy in NHL treatment. Two anti-CD20 mAbs, ibritumomab tiuxetan radiolabeled with yttrium-90 (Zevalin, Spectrum Pharmaceuticals, USA) and tositumomab radiolabeled with iodine-131 (Bexxar, GlaxoSmithKline, UK), were approved for NHL treatment by FDA in 2002 and 2003, respectively. However, Zevalin and Bexxar are used after several rounds of treatment with rituximab, and the remaining circulating rituximab may impair the efficacy of subsequent anti-CD20 therapies.

Therefore, a conjugate that targets a different antigen could be desirable. Lutetium-177 [¹⁷⁷Lu]-lilotomab satetraxetan (Betalutin®, previously known as ¹⁷⁷Lu-DOTA-HH1) is a novel conjugate in which the murine mAb lilotomab targets CD37 receptors expressed on malignant B-cells, and ¹⁷⁷Lu is a beta-emitter with a mean beta energy of 0.133 MeV (mean and max beta-range in water: 0.23 and 1.9 mm). CD37 is a 31 kDa transmembrane protein that belongs to the tetraspanin family. It has a bivalent role on the phosphatidylinositol 3′-kinase (PI3K)/AKT survival pathway and of humoral immunity. As CD37 is highly expressed in NHL cells, it represents an attractive molecule for targeted therapy.

¹⁷⁷Lu-lilotomab is currently tested in a clinical phase 2b trial for the treatment of relapsed indolent B-cell NHL with promising safety and efficacy data especially in patients with FL. An enhancement of the effect of ¹⁷⁷Lu-lilotomab would be very valuable.

### SUMMARY OF THE INVENTION

The present invention relates to a composition comprising a radioimmunoconjugate comprising a monoclonal HH1 antibody, such as ¹⁷⁷Lu-lilotomab, and a protein or molecule capable of leading to progression of the cell cycle through the G2/M checkpoint, a composition comprising a radioimmunoconjugate comprising a monoclonal HH1 antibody, such as ¹⁷⁷Lu-Lilotomab, and a protein or molecule capable of inhibiting progression through Mitosis, a composition comprising a radioimmunoconjugate comprising a monoclonal HH1 antibody, such as ¹⁷⁷Lu-lilotomab, and a protein or molecule capable of inhibition poly ADP ribose polymerase (PARP), and a composition comprising a radioimmunoconjugate comprising a monoclonal HH1 antibody, such as ¹⁷⁷Lu-lilotomab, and a BCL2 protein inhibitor.

These compositions can be used as a medicament.

An aspect of the invention also relates to a combination of a radioimmunoconjugate comprising a monoclonal HH1 antibody, such as ¹⁷⁷Lu-lilotomab, and a protein or molecule which is a BCL2 inhibitor, a protein or molecule capable of inhibiting progression through Mitosis, or a protein or molecule capable of leading to progression of the cell cycle through the G2/M checkpoint, for use as a medicament.

An aspect of the invention also relates to a combination of a radioimmunoconjugate comprising a monoclonal HH1 antibody, such as ¹⁷⁷Lu-lilotomab, and a protein or molecule capable of inhibition poly ADP ribose polymerase (PARP). The inhibition of PARP decreases the cells capability to repair damaged DNA and increase the cells sensitivity ionizing radiation.

The medicament may be against Non-Hodgkin's lymphoma (NHL), and the NHL can be selected from the group consisting of transformed follicular lymphoma, diffuse large B-cell lymphoma, mantle cell lymphoma, marginal zone lymphoma, chronic lymphatic leukemia, cutaneous T-cell lymphoma, lymphoplasmacytic lymphoma, marginal zone B-cell lymphoma, MALT lymphoma, small cell lymphocytic lymphoma, Burkitt lymphoma, anaplastic large cell lymphoma, lymphoblastic lymphoma, peripheral T-cell lymphoma, transplant induced lymphoma.

One embodiment of the present invention is the use in a combination therapy where the composition is followed by simultaneous or post-treatment with antibody therapy, immunoconjugate therapy or a combination thereof. The composition can be followed by anti-CD20 antibody therapy in a single administration or in a repeated administration pattern, and the anti-CD20 antibody can be rituximab, obinutuzumab or ofatumumab.

The radioimmunoconjugate comprising a monoclonal HH1 antibody, such as ¹⁷⁷Lu-lilotomab, may be linked through a chelating linker, which can be selected from the group consisting of p-SCN-benzyl-DOTA, DOTA-NHS-ester, p-SCN-Bn-DTPA and CHX-A″-DTPA.

The protein or molecule can be an inhibitor of proteins involved in G2/M cell cycle arrest.

In one embodiment of the present invention is the protein or molecule selected from the group consisting of MK-1775, PD-166285, AMG 900, AT7519, AZD7762, CYC116, flavopiridol, GSK461364, Alisertib, BI2536, JNJ-7706621, LY2603618, NSC 23766, NU6027, PHA-793887, Tosyl-L-Arginine Methyl Ester (TAME), BI6727 (Volasertib), ON-01910 (Rigosertib), HA-1077 (Fasudil), SCH727965 (Dinaciclib), LY2835219, LEE011, Salirasib, K-115 (Ripasudil), PD0332991 (Palbociclib).

The composition can be formulated as a pharmaceutical composition, which may comprise one or more pharmaceutically acceptable carriers or adjuvants.

### DETAILED DESCRIPTION OF THE INVENTION

The object of the present invention is to identify molecular mechanisms involved in the therapeutic response to a radioimmunoconjugate comprising a monoclonal HH1 antibody, such as ¹⁷⁷Lu-lilotomab, ¹⁷⁷Lu-chHH1.1, and/or ²¹²Pb-chHH1.1, in order to identify i) the NHL type(s) that could most benefit from this treatment and ii) relevant combination partners.

Thus, the present invention relates to a composition comprising a radioimmunoconjugate, such as ¹⁷⁷Lu-lilotomab, ¹⁷⁷Lu-chHH1.1, and/or ²¹²Pb-chHH1.1 for use in the treatment of a specific NHL type cancer.

The present invention also relates to a composition comprising a radioimmunoconjugate, such as ¹⁷⁷Lu-lilotomab, ¹⁷⁷Lu-chHH1.1, and/or ²¹²Pb-chHH1.1, and an additional drug which can be: a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor.

A further aspect relates to the combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, a protein or molecule which is a BCL2 inhibitor, a protein or molecule capable of inhibiting progression through Mitosis, or a protein or molecule which is a PARP inhibitor, for use as a medicament.

### Radioimmunoconjugate

The radioimmunoconjugates of the present invention comprises an antibody and a radionuclide. These may be linked through a linker.

The monoclonal antibody (mAb or moAb) lilotomab was previously known as tetulomab or HH1 while ¹⁷⁷Lu-lilotomab satetraxetan was previously known as ¹⁷⁷Lu-labeled HH1 antibody, or named ¹⁷⁷Lu-tetulomab or by the tradename Betalutin.

Specific variants of HH1 are disclosed in PCT/IB2012/057230 and PCT/EP2011/051231 which hereby are incorporated by reference and disclosed as specific embodiments that are included in this invention. The variable sequences of HH1 are disclosed on page 30-31 (SEQ ID Nos: 1-4) of PCT/IB2012/057230.

It will therefore be possible to adjust the variant of HH1 included in the radioimmunoconjugates of the present invention based on the above-mentioned disclosures. In a preferred embodiment of the present invention is the murine variant or the chimeric variant of HH1. In another embodiment of the present invention is the chimeric variant of HH1 chHH1.1 which is chimeric HH1 isotype IgG1, as disclosed in Example 1 of PCT/IB2012/057230, or chHH1.3H which is chimeric HH1 isotype IgG3 with R435H mutation.

Thus, ¹⁷⁷Lu-lilotomab may refer to Betalutin where the antibody is murine HH1, but can also in another embodiment refer to where the antibody is the chimeric variant chHH1.1.

¹⁷⁷Lu-lilotomab satetraxetan is a radioimmunoconjugate (RIC) also known as antibody radionuclide conjugate (ARC) that is capable of binding to or targeting an antigen of interest. In the present case is this antigen CD37. Satetraxetan is a derivative of DOTA, p-SCN-benzyl-DOTA.

¹⁷⁷Lu-lilotomab may be linked through a chelating linker. The chelating linker selected from the group consisting of p-SCN-benzyl-DOTA, DOTA-NHS-ester, p-SCN-Bn-DTPA, p-SCN-benzyl-TCMC and CHX-A″-DTPA. In one embodiment of the chelating linker satetraxetan, also known as p-SCN-benzyl-DOTA. In this specific embodiment is ¹⁷⁷Lu-lilotomab the drug Betalutin.

In an embodiment of the present invention is the radioimmunoconjugate ¹⁷⁷Lu-chHH1.1. In another embodiment of the present invention is the radioimmunoconjugate ²¹²Pb-chHH1.1. In this specific embodiment p-SCN-benzyl-TCMC is the chelator. chHH1.1 is the chimeric IgG₁ version of the HH1 (lilotomab) antibody.

The radionuclide may therefore be ¹⁷⁷Lu or ²¹²Pb.

Thus, one embodiment of the present invention relates to the combination of a radioimmunoconjugate comprising a monoclonal HH1 antibody, such as ¹⁷⁷Lu-lilotomab, ¹⁷⁷Lu-chHH1.1, and/or ²¹²Pb-chHH1.1 and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, for the use according to the present invention, wherein ¹⁷⁷Lu-lilotomab are linked through a chelating linker.

Another embodiment of the present invention relates to the combination of a radioimmunoconjugate comprising a monoclonal HH1 antibody, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, for the use according to the present invention, wherein the chelating linker selected from the group consisting of p-SCN-benzyl-DOTA, DOTA-NHS-ester, p-SCN-Bn-DTPA, CHX-A″-DTPA and p-SCN-benzyl-TCMC.

Venetoclax blocks the anti-apoptotic B-cell lymphoma-2 (Bcl-2) protein, leading to programmed cell death. Overexpression of Bcl-2 in some lymphoid malignancies has sometimes shown to be linked with increased resistance to chemotherapy. The BCL2 inhibitor can therefore be venetoclax.

The chelators p-SCN-benzyl-DOTA, DOTA-NHS-ester, p-SCN-Bn-DTPA, CHX-A″-DTPA are preferred for chelation of ¹⁷⁷Lu while p-SCN-benzyl-TCMC is preferred for chelation of 212Pb.

A further embodiment of the present invention relates to the combination of a radioimmunoconjugate comprising a monoclonal HH1 antibody, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, for the use according to the present invention, wherein the chelating linker is satetraxetan, also known as p-SCN-benzyl-DOTA.

### Administration Route and Patterns

By administration of radioimmunoconjugate is meant intravenous infusion or intravenous injection. More specifically, the radioimmunoconjugate and antibody of the present invention can be administered directly in a vein by a peripheral cannula connected to a drip chamber that prevents air embolism and allows an estimate of flow rate into the patient. In one embodiment the radioimmunoconjugate and/or antibody can be administered in a repeated fashion.

In another embodiment the radioimmunoconjugate followed by monoclonal antibody (or immunoconjugate) can both be administered in a repeated fashion.

An embodiment of the present invention relates to the use of the radioimmunoconjugate of the present invention administered in combination with or in addition to other therapy.

In an embodiment of the present invention the other therapies are selected from pretreatment with lilotomab, premedication with antipyretics and antihistamine, chemotherapy, immune checkpoint inhibitors, monoclonal antibody therapy, surgery, radiotherapy, and/or photodynamic therapy.

In another embodiment of the present invention the other therapies are bone marrow transplantation or stem cell transplantation and/or therapy.

In one embodiment of the present invention is the composition for use according to the present invention, wherein the use is for a combination therapy where the composition is followed by simultaneous or post-treatment with antibody therapy, immunoconjugate therapy or a combination thereof. The composition may be followed by anti-CD20 antibody therapy in a single administration or in a repeated administration pattern.

The anti-CD20 antibody can be rituximab. The anti-CD20 antibody can also be obinutuzumab or ofatumumab or a rituximab biosimilar like Rixathon or Truxima.

### Administration Dosages

In the present invention is the radioimmunoconjugate comprising a monoclonal HH1 antibody, such as ¹⁷⁷Lu-lilotomab satetraxetan, used in medicaments that can be used in the treatment of Non-Hodgkin's lymphoma. An embodiment of the present invention relates to ¹⁷⁷Lu-lilotomab satetraxetan administered at a concentration selected from the group consisting of 10, 12.5, 15, 17.5, 20, 25, 30, 35, 40, 45, 50 MBq/kg.

In one embodiment of the present invention is the concentration 15 MBq/kg.

In another embodiment of the present invention is the concentration 17.5 MBq/kg.

In a further embodiment of the present invention is the concentration 20 MBq/kg.

Protein or molecule capable of leading to progression of the cell cycle through the G2/M checkpoint

The protein or molecule capable of leading to progression of the cell cycle through the G2/M checkpoint have the ability of influencing the G2/M checkpoint directly or indirectly. This can for example be by phosphorylation or dephosphorylation of key proteins involved in the cell cycle transition.

In one embodiment of the present invention the protein or molecule leads to lower WEE-1 mediated phosphorylation of cyclin-dependent kinase-1 (CDK1) and progression of the cell cycle through the G2/M checkpoint or inhibiting progression through Mitosis. In another embodiment of the present invention the composition according to the present invention, wherein the protein or molecule leads to lower MYT-1 mediated phosphorylation of cyclin-dependent kinase-1 (CDK1) and progression of the cell cycle through the G2/M cell cycle arrest. In a further embodiment of the present invention the protein or molecule leads to higher CDK7-containing CAK kinase mediated phosphorylation of cyclin-dependent kinase-1 (CDK1). The protein or molecule may also be an inhibitor of an AURORA-kinase (AURA, AURB, AURC or Polo-like Kinase PLK1,2,3,4).

The protein or molecule can also be an inhibitor of proteins involved in G2/M cell cycle arrest, such as proteins involved in the transition from G2 to M phase. However, the protein or molecule may also be an activator of proteins that are a limiting factor in the G2/M cell cycle transition.

The combination of or composition comprising a radioimmunoconjugate comprising a monoclonal HH1 antibody, such as ¹⁷⁷Lu-lilotomab, and a protein or molecule capable of leading to progression of the cell cycle through the G2/M checkpoint or capable of inhibiting progression of the cell cycle through M-phase for the use according to the present invention can comprise one or several proteins or molecules. These may be selected from the group consisting of MK-1775, PD-166285, AMG 900, AT7519, AZD7762, CYC116, flavopiridol, GSK461364, JNJ-7706621, LY2603618, NSC 23766, NU6027, PHA-793887, Tosyl-L-Arginine Methyl Ester (TAME), BI6727 (Volasertib), ON-01910 (Rigosertib), HA-1077 (Fasudil), SCH727965 (Dinaciclib), LY2835219, LEE011, Salirasib, K-115 (Ripasudil), PD0332991 (Palbociclib), BI2536, MLN8237 (Alisertib), or a protein 14-3-3 inhibitor such as difopein.

Thus, the protein or molecule can be MK-1775. The protein or molecule may also be PD-166285. The protein or molecule can also be AMG 900. The protein or molecule can also be AZD7762. The protein or molecule may also be JNJ7706621. The protein or molecule may also be CYC116. The protein or molecule can also be AT7519. The protein or molecule can also be LY2603618. The protein or molecule may also be flavopiridol. The protein or molecule may also be GSK461364. The protein or molecule can also be NSC 23766. The protein or molecule may also be NU6027. The protein or molecule can also be PHA-793887. The protein or molecule may also be Tosyl-L-Arginine. The protein or molecule can also be Methyl Ester (TAME). The protein or molecule can also be BI6727 (Volasertib). The protein or molecule may also be ON-01910 (Rigosertib). The protein or molecule may also be HA-1077 (Fasudil). The protein or molecule can also be SCH727965 (Dinaciclib). The protein or molecule may also be LY2835219. The protein or molecule may also be LEE011. The protein or molecule may also be Salirasib. The protein or molecule may also be K-115 (Ripasudil). The protein or molecule may also be PD0332991 (Palbociclib). The protein or molecule may also be a 14-3-3 inhibitor. The protein or molecule may also be difopein. The protein or molecule may also be PLK1 inhibitor BI2536. The protein or molecule may also be Aurora kinase inhibitor MLN8237 (Alisertib).

A Protein or Molecule which is a PARP Inhibitor (PARPi)

PARP inhibitors are a group of pharmacological inhibitors of the enzyme poly ADP ribose polymerase (PARP). The inhibitors are effective for several indications, including cancers.

The combination of or composition comprising a radioimmunoconjugate comprising a monoclonal HH1 antibody, such as ¹⁷⁷Lu-lilotomab, ¹⁷⁷Lu-chHH1.1, and/or ²¹²Pb-chHH1.1, and a protein or molecule which is a PARP inhibitor for the use according to the present invention can comprise one or several proteins or molecules. These may be selected from the group consisting of olaparib (AZD2281, Ku-0059436), Veliparib (ABT-888), Rucaparib (AG-014699, PF-01367338), Talazoparib (BMN 673), AG-14361, INO-1001 (3-aminobenzamide), A-966492, P334 HCl, Niraparib (MK-4827), UPF 1069, ME0328, NMS-P118, E7449, Picolinamide, benzamide, niraparib (MK-4827) tosylate, NU1025, iniparib (BSI-201), AZD2461, and BGP-15 2HCl.

Thus, the protein or molecule can be olaparib (AZD2281, Ku-0059436). The protein or molecule can also be Veliparib (ABT-888). The protein or molecule can also be Rucaparib (AG-014699, PF-01367338). The protein or molecule can also be Talazoparib (BMN 673). The protein or molecule can also be AG-14361. The protein or molecule can also be INO-1001 (3-aminobenzamide). The protein or molecule can also be A-966492. The protein or molecule can also be P334 HCl. The protein or molecule can also be Niraparib (MK-4827). The protein or molecule can also be UPF 1069. The protein or molecule can also be ME0328. The protein or molecule can also be NMS-P118. The protein or molecule can also be E7449. The protein or molecule can also be Picolinamide. The protein or molecule can also be benzamide. The protein or molecule can also be niraparib (MK-4827) tosylate. The protein or molecule can also be NU1025. The protein or molecule can also be iniparib (BSI-201). The protein or molecule can also be AZD2461. The protein or molecule can also be BGP-15 2HCl.

The combination of or composition comprising a radioimmunoconjugate comprising a monoclonal HH1 antibody, such as ¹⁷⁷Lu-lilotomab, ¹⁷⁷Lu-chHH1.1, and/or ²¹²Pb-chHH1.1, and a protein or molecule which is a BCL2 inhibitor for the use according to the present invention can comprise one or several proteins or molecules. These may be selected from the group consisting of venetoclax (ABT-199, GDC-0199), obatoclax mesylate (GX15-070), HA14(1), ABT-263 (navitoclax), ABT-737, TW-37, AT101, sabutoclax, WEHI-539, A-1155463, gossypolk and AT-101, apogossypol, S1, 2-methoxyantimycin A3, BXI-61, BXI-72, TW37, MIM1, UMI-77, and gambogic acid.

Thus, the protein or molecule can be venetoclax (ABT-199, GDC-0199). The protein or molecule can also be obatoclax mesylate (GX15-070). The protein or molecule can also be HA14(1). The protein or molecule can also be ABT-263 (navitoclax). The protein or molecule can also be TW-37. The protein or molecule can also be AT101. The protein or molecule can also be sabutoclax. The protein or molecule can also be gambogic acid. The protein or molecule can also be WEHI-539. The protein or molecule can also be A-1155463. The protein or molecule can also be gossypol and AT-101. The protein or molecule can also be apogossypol. The protein or molecule can also be S1. The protein or molecule can also be 2-methoxyantimycin A3. The protein or molecule can also be BXI-61. The protein or molecule can also be BXI-72. The protein or molecule can also be TW37. The protein or molecule can also be MIM1. The protein or molecule can also be UMI-77.

### Pharmaceutical Composition

Antibodies, radioimmunoconjugates, and other drugs are usually applied in the treatment of diseases formulated in pharmaceutical compositions. Such compositions are optimized for parameters such as physiological tolerance and shelf-life.

Thus, in one embodiment of the present invention is the radioimmunoconjugates and/or composition of the present invention formulated as one or more pharmaceutical compositions.

An embodiment of the present invention relates to a pharmaceutical composition as described above, further comprising one or more additional therapeutic agents. In another embodiment of the present invention are said one or more additional therapeutic agents selected from agents that induce apoptosis. Usually is an important element of a pharmaceutical composition a buffer solution, which to a substantial degree maintain the chemical integrity of the radioimmunoconjugate and is being physiologically acceptable for infusion into patients.

In one embodiment of the present invention the pharmaceutical composition comprises one or more pharmaceutically acceptable carriers and/or adjuvants. Acceptable pharmaceutical carriers include but are not limited to non-toxic buffers, fillers, isotonic solutions, etc. More specifically, the pharmaceutical carrier can be but are not limited to normal saline (0.9%), half-normal saline, Ringer's lactate, 5% Dextrose, 3.3% Dextrose/0.3% Saline. The physiologically acceptable carrier can contain a radiolytic stabilizer, e.g., ascorbic acid, which protect the integrity of the radiopharmaceutical during storage and shipment.

Preferably are sodium dihydrogen phosphate monohydrate, sodium chloride, recombinant human albumin, sodium ascorbate, diethylenetriamine pentaacetic acid (DTPA) and sodium hydroxide used as excipients in the formulation buffer. Preferably is phosphate included in the formulation buffer to maintain the pH of the finished product during the shelf life.

Preferably is recombinant human albumin included in the formulation buffer as a stabilizer for the lilotomab satetraxetan conjugate. The albumin also acts as a radioprotectant. Recombinant human albumin structurally identical to human serum albumin derived from yeast is used. No human- or animal-derived raw material is involved in its manufacture. The excipient is well known and is used in pharmaceutical products for human use.

Preferably is sodium ascorbate included in the formulation to act as a radiolytic scavenger to ensure the stability of Betalutin over the shelf-life of the product. Preferably is DTPA introduced as an excipient in the Betalutin formulation to chelate any free ¹⁷⁷Lu³⁺ ions and to reroute this impurity from accumulation in the bone to rapid renal clearance (Li et al 2001, Breeman et al 2003). Betalutin contains 9.3 μmol DTPA in 12 mL, while the maximum amount of no-carrier added (n.c.a)¹⁷⁷Lu³⁺ (>3,000 GBq/mg) applied (6.9 GBq) corresponds to less than 15 nmol Lu ions. This gives a more than 1000-fold molar excess of DTPA over Lu³⁺ ions. Furthermore, when taking into account that the majority of the Lu³⁺ ions 95%) is chelated to lilotomab satetraxetan, the molar excess is almost 100,000-fold. DTPA is therefore expected to chelate all free ¹⁷⁷Lu³⁺ ions quantitatively and ¹⁷⁷Lu-DTPA is thus specified as radiochemical impurity in the specification.

Preferably is the formulation buffer an aqueous solution with pH 6.9 to 7.0 and thus no incompatibilities between the drug substance and the formulation buffer are expected. One embodiment of the present invention comprises the pharmaceutical composition of the present invention and one or more additional antibodies or radioimmunoconjugates.

As aspect of the present invention relates to a pharmaceutical composition comprising (per mL): 0.75 mg Lutetium (¹⁷⁷Lu) lilotomab satetraxetan, 0.46 mg Ammonium acetate, and Trace amounts of HCl3. Another aspect of the present invention relates to a pharmaceutical composition comprising (per mL): 30.86 mg Sodium ascorbate, 0.31 mg DTPA, 0.17 mg NaOH, 60.82 mg Recombinant human albumin, 3.32 mg Sodium dihydrogen phosphate monohydrate, and 4.34 mg Sodium chloride with the pH is adjusted to 6.9-7.0.

A further aspect of the present invention relates to a pharmaceutical composition comprising; 14% of the pharmaceutical composition comprising (per mL): 0.75 mg Lutetium (¹⁷⁷Lu) lilotomab satetraxetan, 0.46 mg Ammonium acetate, and Trace amounts of HCl3, and 86% of the pharmaceutical composition comprising (per mL): 30.86 mg Sodium ascorbate, 0.31 mg DTPA, 0.17 mg NaOH, 60.82 mg Recombinant human albumin, 3.32 mg Sodium dihydrogen phosphate monohydrate, and 4.34 mg Sodium chloride with the pH is adjusted to 6.9-7.0.

The present invention also relates to the pharmaceutical compositions of the present examples, as well as the dosage administration patterns presented herein. This includes the use of the pharmaceutical compositions of the present invention for use in the treatment of Non-Hodgkin lymphoma.

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, venetoclax, or the protein or molecule which is a PARP inhibitor, can be used according to the present invention, and wherein the radioimmunoconjugate comprising a monoclonal HH1 antibody, such as ¹⁷⁷Lu-lilotomab, ¹⁷⁷Lu-chHH1.1, and/or ²¹²Pb-chHH1.1, and the protein or molecule may be formulated in one or more pharmaceutical compositions.

The combination of a radioimmunoconjugate, such as ¹⁷⁷Lu-lilotomab, ¹⁷⁷Lu-chHH1.1, and/or ²¹²Pb-chHH1.1 and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, may be used according to the present invention, wherein the pharmaceutical composition comprises one or more pharmaceutically acceptable carriers or adjuvants.

### Medical Applications

The person in need of treatment with a radioimmunoconjugate comprising a monoclonal HH1 antibody, such as ¹⁷⁷Lu-lilotomab satetraxetan, ¹⁷⁷Lu-chHH1.1, and/or ²¹²Pb-chHH1.1 is suffering from a CD37 related disease, typically a B-cell lymphoma such as Non-Hodgkin lymphoma (NHL).

NHL is a group of blood cancers that includes all types of lymphoma except Hodgkin's lymphomas. Symptoms include enlarged lymph nodes, fever, night sweats, weight loss, and feeling tired. Other symptoms may include bone pain, chest pain, or itchiness. Some forms are slow growing while others are fast growing. There are several types of NHL. Thus, another embodiment of the present invention relates to the lymphoma being a subtype selected from the group consisting of follicular grade I-IIIA, marginal zone, small lymphocytic, lymphoplasmacytic, Diffuse large B-cell lymphoma, and mantle cell.

Thus, the radioimmunoconjugate, compositions of the present invention and/or the combination of the present invention can be used as a medicament. The medicament can be against Non-Hodgkin's lymphoma (NHL).

The NHL may be selected from the group consisting of transformed follicular lymphoma, diffuse large B-cell lymphoma, mantle cell lymphoma, marginal zone lymphoma, chronic lymphatic leukemia, cutaneous T-cell lymphoma, lymphoplasmacytic lymphoma, marginal zone B-cell lymphoma, MALT lymphoma, small cell lymphocytic lymphoma, Burkitt lymphoma, anaplastic large cell lymphoma, lymphoblastic lymphoma, peripheral T-cell lymphoma, transplant induced lymphoma.

Thus, for the composition for use according to the present invention, or for the combination for use according to the present invention, the NHL may be transformed follicular lymphoma. For the composition for use according to the present invention, or for the combination for use according to the present invention, the NHL may be diffuse large B-cell lymphoma. In one embodiment of the present invention is the NHL mantle cell lymphoma. In another embodiment of the present invention is the NHL marginal zone lymphoma. In a further embodiment of the present invention is the NHL chronic lymphatic leukemia. In yet another embodiment of the present invention is the NHL cutaneous T-cell lymphoma. In one embodiment of the present invention is the NHL lymphoplasmacytic lymphoma. In one embodiment of the present invention is the NHL marginal zone B-cell lymphoma. In another embodiment of the present invention is the NHL MALT lymphoma. In yet another embodiment of the present invention is the NHL small cell lymphocytic lymphoma. In one embodiment of the present invention is the NHL Burkitt lymphoma. In another embodiment of the present invention is the NHL anaplastic large cell lymphoma. In one embodiment of the present invention is the NHL lymphoblastic lymphoma. In another embodiment of the present invention is the NHL peripheral T-cell lymphoma. In a further embodiment of the present invention is the NHL transplant induced lymphoma.

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, can be for use according to the present invention, wherein the medicament is against Non-Hodgkin's lymphoma.

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, can be for use according to the present invention, wherein the use is for a combination therapy where the composition is followed by simultaneous or post-treatment with antibody therapy, immunoconjugate therapy or a combination thereof.

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor, can be for use according to the present invention, wherein the composition is followed by anti-CD20 antibody therapy in a single administration or in a repeated administration pattern. The anti-CD20 antibody can be rituximab, or obinutuzumab or ofatumumab.

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, or a protein or molecule capable of inhibiting progression through Mitosis, can be for use according to the present invention, wherein the protein or molecule leads to lower WEE-1 mediated phosphorylation of cyclin-dependent kinase-1 (CDK1).

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, or a protein or molecule capable of inhibiting progression through Mitosis, can be for use according to the present invention, wherein the protein or molecule leads to lower MYT-1 mediated phosphorylation of cyclin-dependent kinase-1 (CDK1).

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint, or a protein or molecule capable of inhibiting progression through Mitosis, a protein or molecule which is a BCL2 inhibitor, can be for use according to the present invention, wherein the protein or molecule leads to higher CDK7-containing CAK kinase mediated phosphorylation of cyclin-dependent kinase-1 (CDK1).

The combination of a radioimmunoconjugate, such as ¹⁷⁷Lu-lilotomab, ¹⁷⁷Lu-chHH1.1, and/or ²¹²Pb-chHH1.1, and an additional drug which can be a protein or molecule capable of leading to progression of the cell cycle through the G2/M checkpoint can be for use according to the present invention, wherein the protein or molecule is an inhibitor of G2/M cell cycle arrest, a protein or molecule capable of inhibiting progression through Mitosis, a protein or molecule which is a BCL2 inhibitor, or a protein or molecule which is a PARP inhibitor.

The protein or molecule capable of leading to progression of the cell cycle through the G2/M checkpoint can specifically target enzymes that are involved in CDK1 T14 phosphorylation. The protein or molecule capable of leading to progression of the cell cycle through the G2/M checkpoint can specifically target enzymes that are involved in CDK1 Y15 phosphorylation. The protein or molecule capable of leading to progression of the cell cycle through the G2/M checkpoint can specifically target enzymes that are involved in CDK1 Y161 phosphorylation.

The combination may be in the comprised in the same composition, or seen as a combinational treatment where the compounds are administered separately.

Certain types of cancer have specific traits where the use of radioimmunoconjugates can be beneficial. These include cancer types where G2/M cell cycle arrest is inhibited. The cancer type may also be where G1 cell cycle arrest is inhibited.

Thus, one aspect of the present invention relates to a composition comprising ¹⁷⁷Lu-lilotomab satetraxetan for use in the treatment of Non-Hodgkin's lymphoma showing reduced inhibitory CDK1 phosphorylation. The reduced inhibitory CDK1 phosphorylation can be from lower WEE-1 mediated phosphorylation of cyclin-dependent kinase-1 (CDK1). The reduced inhibitory CDK1 phosphorylation can be from lower MYT-1 mediated phosphorylation of cyclin-dependent kinase-1 (CDK1).

Another aspect of the present invention relates to composition comprising ¹⁷⁷Lu-lilotomab satetraxetan for use in the treatment of Non-Hodgkin's lymphoma showing higher CDK7-containing CAK kinase mediated phosphorylation of cyclin-dependent kinase-1 (CDK1).

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### BRIEF DESCRIPTION OF THE FIGURES

FIG. 1 shows apoptosis measurement in Ramos, DOHH2 and Rec-1 cells, after exposure for 18 h to unlabelled mAbs (treatment period: bars below x-axis).

FIG. 2 shows apoptosis measurement in Ramos, DOHH2 and Rec-1 cells, after exposure to 6 MBq/mL of radiolabelled mAbs for 18 h (treatment period: bars below x-axis).

FIG. 3 shows distribution of Ramos, DOHH2 and Rec-1 cells in cell cycle phases, after exposure for 18 h to 0 or 6 MBq/mL of ¹⁷⁷Lu-lilotomab or ¹⁷⁷Lu-rituximab (treatment period: bars below x-axis).

FIG. 4 shows distribution of Ramos, DOHH2 and Rec-1 cells in cell cycle phases, after exposure for 18 h to 0 or 40 μg/mL of lilotomab or rituximab (treatment period: bars below x-axis)

FIG. 5 shows expression of CDK1 and its phosphorylations in Ramos, DOHH2 and Rec-1 cells at different time after exposure to 6 MBq/mL of ¹⁷⁷Lu-lilotomab for 18 h.

FIG. 6 shows expression of p345-Chk1, Wee-1, Myt-1 and CDK7 in Ramos, DOHH2 and Rec-1 cells at different time after exposure to 6 MBq/mL of ¹⁷⁷Lu-lilotomab for 18 h.

FIG. 7 shows effect of inhibitors of G2/M arrest on cell proliferation.

FIG. 8 shows expression of p14-CDK1 and p15-CDK1 in Ramos, DOHH2 and Rec-1 cells after treatment for 18 h with ¹⁷⁷Lu-lilotomab+MK-1775 or ¹⁷⁷Lu-lilotomab+PD-166285.

FIG. 9 shows ratio between the number of treated cells in G2/M and the number of untreated cells in G2/M. Treatments were ¹⁷⁷Lu-lilotomab alone or combined with MK-1775 or PD-166285.

FIG. 10 shows proposed mechanism of action of ¹⁷⁷Lu-lilotomab.

FIG. 11 shows quantification of CD133 and CD44 at the Ramos, DOHH2 and Rec-1 cell surface after RIT (treatment period: bar below x-axis).

FIG. 12 shows Combination Index calculated using the Chou-Talalay method for combination of 0.5 μg/ml or 1 μg/ml Humalutin with different doses of olaparib.

FIG. 13 shows proliferation of of non-treated (Minus_Betalutin) and Betalutin treated U2932 (A,B) and RIVA (C,D) cells, treated with 1 μg/ml (U-2932) and 0.5 μg/ml (RIVA) Betalutin, respectively (Plus Betalutin). Three days after seeding RealTimeGlo was added to the media and relative luminescence units (RLU) measured on 3 consecutive days starting at day 3 (A,C). Error bars depict standard deviation of replicates. The assay measures a luminescent signal that is dependent upon continuous reduction of the MT cell viability substrate by viable cells and rapid turnover by NanoLuc Luciferase. Panels B and D show relative luminescence measured normalized to start of observation period (day 3).

FIG. 14 shows relative proliferation of treated U-2932 (2A) and RIVA (2B) at respective days 3, 4, 5, and 6 (relative cell growth.

FIG. 15 shows relative proliferation of treated U-2932 (A,B) and RIVA (C,D) cells in respect to untreated control cells at days 3, 4, 5, and 6. AG-14361 had only a minor inhibitory effect on cell proliferation of U-2932 or RIVA cells in absence of Betalutin when used at 10 nM (A,B). AG-14361 potentiated the proliferation inhibitory effect of Betalutin in U-2932 cells at 1 μM f.c. (C) and RIVA cells, when co-administered at 100 nM f.c. (D).

FIG. 16 shows relative proliferation of treated U-2932 (A,B) and RIVA (C,D) cells in respect to untreated control cells at days 3, 4, 5, and 6. Rucaparib had no inhibitory effect on cell proliferation of U-2932 or RIVA cells in absence of Betalutin at all tested concentrations. Rucaparib potentiated the proliferation inhibitory effect of Betalutin in U-2932 cells at 1 μM f.c. (C) and RIVA cells, when co-administered at 100 nM f.c. (D). FIG. 17 shows BLISS scores for combinations of Betalutin and PARP inhibitors AG-14361 (right pillar) or Rucaparib (middle pillar) in U-2932 (A) and RIVA (B). Left pillars indicate the arbitrary cut-off of two standard deviations of the effect of Betalutin single agent treatment measured at the indicated days.

FIG. 18 shows Combination Index calculated using the Chou-Talalay method for combination of 0.5 μg/ml and 1 μg/ml Humalutin with different doses of venetoclax.

FIG. 19 shows effect of inhibitors of G2/M arrest on cell cycle in vitro and on tumor progression in vivo. (A) The ratio of Ramos, DOHH2, Rec-1 and OCI-Ly8 cells in the G2/M cell cycle upon exposure to ¹⁷⁷Lu-lilotomab or ¹⁷⁷Lu-lilotomab+1 μM MK-1775 (WEE-1 inhibitor) or PD-166285 (WEE-1 and MYT-1 inhibitor) versus untreated cells (Ctrl; set to 1) was determined. Data are the mean±SD of three independent experiments in triplicate. (B) Athymic mice bearing Ramos tumor xenografts received i) one intravenous injection of ¹⁷⁷Lu-lilotomab at 250 MBq/kg or 500 MBq/kg, ii) 30 mg/kg MK-1775 (twice a day) from day 1 to 5 post-injection iii) combination of ¹⁷⁷Lu-lilotomab at 250 MBq/kg+30 mg/kg MK-1775 (twice a day) from day 1 to 5 post-injection. Tumor growth (left panel) was monitored as a function of time post-xenograft and Kaplan Meyer survival curves were established (right panel), (n=6-9/group). (C) Athymic mice bearing OCI-Ly8 tumor xenografts received i) one intravenous injection of ¹⁷⁷Lu-lilotomab at 250 MBq/kg or 500 MBq/kg ii) 30 mg/kg MK-1775 (twice a day) from day 1 to 5 post-injection iii) combination of ¹⁷⁷Lu-lilotomab at 250 MBq/kg+30 mg/kg MK-1775 (twice a day) from day 1 to 5 post-injection iv) unlabeled mAbs (0.5 mg/kg). Tumor growth (left panel) was monitored as a function of time post-xenograft and Kaplan Meyer survival curves were established (right panel) (n=6-8/group).

FIG. 20 shows efficacy of ¹⁷⁷Lu-lilotomab combined or not with inhibitors G2/M cell cycle arrest on cells grown from patient biopsies. (A) Fluorescence-activated cell sorting analysis of the proportion of CD3−/CD20+ cells in biopsies of patients with DLBCL or FL. Cells were exposed for 18 h to ¹⁷⁷Lu-lilolotmab and analysis was done 3 days later (day 4). (B) Theoretical (using Bliss independence mathematical model) and experimental additive anti-proliferative effects of ¹⁷⁷Lu-lilotomab and MK-1775 or PD-166285 after 18 h cells exposure. Analysis was done on day 1 or 4 post treatment.

FIG. 21: (A) Proliferation potential of U-2932 and RIVA cells treated with Betalutin alone or in combination with JNJ-7706621 at indicated dose. Proliferation potential is given normalized to untreated control cells (see also Example 2). (B) Bar-diagram depicting BLISS independence score of indicated combination (observed effect size substracted by expected additive effect size). Light grey bars indicate cut-off for significant surpass of additivity as defined as a surpass larger than twice the standard deviation of the effect of Betalutin alone.

FIG. 22: (A,B) Proliferation potential of U-2932 and RIVA cells treated with Betalutin alone or in combination with PLK1 inhibitors GSK461364 (A) or BI2536 (B) at indicated dose. Proliferation potential is given normalized to untreated control cells (see also Example 2). (C,D) Bar-diagram depicting BLISS independence score of indicated combination (observed effect size substracted by expected additive effect size). Bars to the right for each day indicate cut-off for significant surpass of additivity as defined as a surpass larger than twice the standard deviation of the effect of Betalutin alone.

FIG. 23: (A) Proliferation potential of U-2932 and RIVA cells treated with Betalutin alone or in combination with Aurora B kinase inhibitor MLN8237 (Alisertib) at indicated dose. Proliferation potential is given normalized to untreated control cells (see also Example 2). (B) Bar-diagram depicting BLISS independence score of indicated combination (observed effect size substracted by expected additive effect size). Light grey bars indicate cut-off for significant surpass of additivity as defined as a surpass larger than twice the standard deviation of the effect of Betalutin alone.

FIG. 24: Absolute (left panel) and relative (right panel) proliferation curves of U-2932 cells between days 3 and 6 treated or not with 0.5, 1, or 2 μg/ml Betalutin. Error bars depict standard deviation (n=3).

FIG. 25: Average effect (Fa)-Combination index-plots and dose-response curves of U-2932 cells treated with indicated combinations of Betalutin and BI2536 (A) or GSK461364 (B). Proliferation was assessed at day 5 and normalized to untreated control cells. Error bars depict standard deviation (n=3).

FIG. 26: Average effect (Fa)-Combination index-plots and dose-response curves of U-2932 cells treated with indicated combinations of Betalutin and MLN8237 (Alisertib). Proliferation was assessed at day 5 and normalized to untreated control cells. Error bars depict standard deviation (n=3).

FIG. 27: Average effect (Fa)-Combination index-plots and dose-response curves of U-2932 cells treated with indicated combinations of Betalutin and JNJ-7706621. Proliferation was assessed at day 5 and normalized to untreated control cells. Error bars depict standard deviation (n=3).

FIG. 28: Average effect (Fa)-Combination index-plots of three independent experiments of U-2932 cells treated with Betalutin and JNJ-7706621. U-2932 validation plot is as in FIG. 27 and shown for comparison with plots from confirmation experiments. Ranges of antagonism (CI >1.1), additivity (CI 0.9-1.1), and synergism (CI<0.9). Grades of grey shading define ranges of very strong synergism (CI<0.1), strong synergism (CI 0.3-0.1), synergism (CI 0.7-0.3), moderate synergism (CI 0.85-0.7).

FIG. 29: Drug response curves and fittings of venetoclax and humalutin alone and the combination of both treatments.

FIG. 30: Drug response curves and fittings of olaparib and humalutin alone and the combination of both treatments.

### EXAMPLES

### Example 1

The advantage of ¹⁷⁷Lu-lilotomab over rituximab in B-cell non-Hodgkin lymphoma involves modulation of radiation-mediated G2/M cell cycle arrest

### Materials and Methods

### I. B-Cell Lymphoma Models, Monoclonal Antibodies and Animal Models

### A. Cell Lines

Ramos, DOHH2 and Rec-1 cell lines were obtained from ATCC (American type culture collection) and ECACC (European collection of authenticated cell cultures). They express CD20 and CD37 antigens and could then be targeted with rituximab and lilotomab, respectively. The cells were grown between 2-10×10⁵ cells/mL at 37° C. in a humidified atmosphere of 95% air/5% CO₂ in RPMI supplemented with 10% heat-inactivated foetal bovine serum, 100 μg/ml of L-glutamine, and antibiotics (0.1 U/ml penicillin and 100 μg/ml streptomycin). They were routinely tested for mycoplasma contamination using the Mycotest assay (Life Technologies).

Ramos cell line was collected from a Burkitt's lymphoma of a 3-year-old boy. These cells are characterised by the expression of IgMλ and the presence of the t(8,14) translocation overexpressing c-Myc oncogene.

DOHH2 cell line was established from the pleural effusion of a 60-year-old man with refractory immunoblastic B cell lymphoma progressed from follicular centroblastic/centrocytic lymphoma (follicular lymphoma derived of GC). This cell line is characterised by the secretion of IgGλ and by the athypic presence of the t(14;18)(q32;q21) and t(8;14)(q24;q32) translocations leading to an overexpression of c-Myc and also Bcl-2. This anomaly induces that the DOHH2 cell line is a transformed FL (follicular lymphoma) progressing to transformed DLBCL (diffuse large B cell lymphoma).

Rec-1 cell line was established from the lymph node or peripheral blood from a 61-year-old man with terminal DLBCL progressing to transformed mantle lymphoma. This cell line is characterised by the presence of the t(11;14)(q13;q32) overexpressing the cyclin D1.

### B. Antibodies

Rituximab is a chimeric anti-CD20 IgG1 recognising the epitope (170)ANPS(173) and (182)YCYSI(186), with a nanomolar equilibrium dissociation constant. This mAb is developed by Roche (Basel, Switzerland) under the trademark name MabThera® in Europe.

The lilotomab is a murine anti-CD37 IgG1 mAb directed against the epitope 206HLARSRH212 of the CD37 receptor, with a nanomolar equilibrium dissociation constant. This mAb is developed by Nordic Nanovector ASA (Oslo, Norway) and commercialised as Lutetium-177 [¹⁷⁷Lu]-lilotomab satetraxetan (Betalutin®, previously known as ¹⁷⁷Lu-DOTA-HH1).

The cetuximab (Erbitux®, Merck KGaA, Darmstadt, Germany) has been used as non-specific mAb. This mAb is directed again the epidermal growth factor receptor (EGFR) which is highly expressed in many cancers but not in the NHL cells. In this project, it was used radiolabelled with ¹⁷⁷Lu to investigate the radiation-induced effects of ¹⁷⁷Lu alone since it did not bind any of the three B-cell lymphoma models used.

### C. Animal Models

Athymic Nude-Foxn1 mice (6 weeks/old female) and C.B-17/IcrHanHsd-Prkdc-scid mice (6 weeks/old female) from Envigo (Gannat, France) were used. Mice acclimated for 1 week before experimental use. They were housed at 22° C. and 55% humidity with a light-dark cycle of 12 h. They were maintained under pathogen-free conditions and food and water were supplied ad libitum.

### II. In Vitro Studies

### A. Monoclonal Antibody Radiolabelling

MAbs (rituximab, lilotomab and the non-specific cetuximab) conjugated with p-SCN-benzyl-DOTA were obtained from Nordic Nanovector at a concentration of 10 mg/mL and were maintained at 4° C. ¹⁷⁷LuCl₃ was obtained from Perkin Elmer at a volumic activity of 370 MBq in 8 μL of 0.05 M HCl and at specific activity >740 GBq/mg. Radiochemical purity was >97% with radionuclidic purity >99.94%. Arbitrarily, mAbs were labelled with ¹⁷⁷Lu at a specific activity of 200 MBq/mg. Typically, 10 μl of 10 mg/mL DOTA-mAb were mixed with 25 μl 0.25 M NH4OAc (pH 5.5) and pre-heated for 5 min at 37° C. 1 μl of ¹⁷⁷LuCl₃ was added to the reaction mixture (200 MBq/mg) and incubated further at 37° C. for 45 min. Reaction was stopped by adding formulation buffer (100 μL) (PBS, 7.5% BSA, 1 mM DTPA, pH 7.5). Reaction mixture was purified with a PD-10 column (GE Healthcare UK Ltd., Buckinghamshire, England) with PBS as the eluate. Radiochemical purity was determined by applying 1 μl of the reaction onto thin layer chromatography (TLC) and separation was done in a migration vial containing 1 ml of PBS. The strip was cut in two and the activity of each part was measured in a gamma detector. The radiolabelling yield was obtained by dividing the value for the lower part by the total value. It was generally above 99%. Yield was determined as the ratio between activity of ¹⁷⁷Lu added and activity of ¹⁷⁷Lu-mAbs collected. Immunoreactivity of ¹⁷⁷Lu-mAbs was determined using binding assay. Typically, 4 counting tubes containing at least 16×10⁶ Ramos cells in 200 μL PBS/BSA (0.5%) were used. Two tubes were treated with 20 μg of unlabelled mAbs. 15 min later, 10 ng of radiolabelled mAb were added into the 4 tubes and incubated 1 h at room temperature. Radioactivity was measured with a gamma counter before and after washing (three times with PBS-BSA 0.5%). Immunoreactivity was defined as the ratio between bound/total of radioactivity (%).

### B. Determination of the Number of CD20 and CD37 Receptors at the Surface of Ramos, DOHH2 and Rec-1 Cells

The number of CD20 and CD37 receptors at the surface of Ramos, DOHH2 and Rec-1 cells was determined using Scatchard binding assay. Typically, 1×10⁶ Ramos, DOHH2 and Rec-1 cells grown in tubes containing 100 μL culture medium were incubated with increasing amount (0-6.25 nM; average specific activity of 200 MBq/mg) of radiolabelled mAbs (lilotomab or rituximab) for 1 h at room temperature. Radioactivity was next gamma-counted and the cells were washed twice with PBS in order to remove unbound radioactivity. Cells were next resuspended in 1 mL of culture medium and an aliquot fraction was used for cells numbering and bound radioactivity was next determined. The ratio between bound and free radioactivity was determined. It was next expressed as a function of bound radioactivity.

The Scatchard method allows the calculation of the dissociation constant (Kd) and the total number of antigen. Indeed, a determined number of cells is placed in presence of increase concentration of radiolabelled mAb. For each concentration, the ratio bound/free activity is calculated. Finally, a Scatchard plot is traced, corresponding to bound activity as a function of the ratio bound/free activity. Knowing the number of cells in each well and the characteristics of the mAb radiolabelling (number of ¹⁷⁷Lu per mAb), the number of receptors per cell and the Kd could be calculated.

### C. Determination of the Therapeutic Efficacy of ¹⁷⁷Lu-mAbs on B-Cell Lymphomas

Therapeutic efficacy of ¹⁷⁷Lu-lilotomab, ¹⁷⁷Lu-rituximab and ¹⁷⁷Lu-cetuximab was investigated using clonogenic assay. Since cells were in suspension, we developed a protocol using MethoCult® medium (H4435, Stemcell technologies) a methylcellulose medium with recombinant cytokines and EPO for human cells. A concentration of 1×10⁶ Ramos and DOHH2 cells/mL were grown in 12 micro-well plates containing 1 mL of RPMI medium before being incubated with increasing activities (0; 0.5; 1; 2; 4 and 6 MBq/mL) of ¹⁷⁷Lu-labelled mAbs (lilotomab, rituximab or irrelevant cetuximab) for 18 h at 37° C./5% CO₂. Next, cells were collected and centrifuged and washed twice with medium before being resuspended in 5 mL of RPMI medium and counted. From 1500 to 45000 cells were mixed with 4.5 mL of MethoCult® medium and seeded (1.5 mL/dish). The number of seeded cells/dish range between 500 and 15 000, depending on the mAb and on the test activity. Petri dishes were next kept for 12 to 16 days for determining the number of colonies. Colonies containing 50 or more cells were scored and the surviving fraction was calculated. All the experiments were repeated at least three times in triplicate. Using this approach, the cytotoxicity of mAbs or radiolabelled mAbs that could kill cells within the first 18 h was not taken into account.

D. Molecular Mechanisms Involved in Cell Response after RIT

### 1. Cell Cycle Analyse

Cell cycle arrest was assessed in 1×10⁶ Ramos, DOHH2 or Rec-1 cells/mL grown in 12-well plates and exposed for 18 h to 0, 2 (data not shown) and 6 MBq/mL of ¹⁷⁷Lu-lilotomab, ¹⁷⁷Lu-rituximab and ¹⁷⁷Lu-cetuximab or with corresponding amounts of unlabelled mAbs (0, 15 and 40 μg/mL). Cells were harvested at day 0, 2 h, 18 h, 1 d, 2 d, 3 d, of RIT then fixed in 70% ethanol at −20° C. for 3 hours and stained with Cell Cycle kit (with PI) reagent from Merck Millipore in the dark for 30 min at room temperature before analysis using a Muse® flow cytometer. The percentage of cells in G0/G1, S and G2/M phases was then calculated (mean of three experiments in triplicate).

### 2. Apoptosis Induction

Apoptosis induction was assessed in 1×10⁶ Ramos, DOHH2 or Rec1 cells/mL grown in 12-well plates and exposed for 18 h to 0, 2 (data not shown) and 6 MBq/mL of ¹⁷⁷Lu-lilotomab, ¹⁷⁷Lu-rituximab, ¹⁷⁷Lu-cetuximab or with corresponding amounts (0, 15 and 40 μg/mL) of unlabelled mAbs. Cells were harvested at 0, 2 h, 18 h, 1 d, 2 d, 3 d of RIT. At each time point, apoptosis was detected with cell cycle kit reagent from Merck Millipore (Annexin V Kit with 7-AAD) in the dark for 30 min at room temperature before analysis using Muse® flow cytometer

### 3. Analysis of Protein Expressions by Western Blot

Protein expression was assessed in 1×10⁶ Ramos, DOHH2 or Rec-1 cells/mL grown in 12-well plates and exposed for 18 h to 0 and 6 MBq/mL of ¹⁷⁷Lu-lilotomab. Cells were harvested at 0, 2 h, 18 h, 1 d, 2 d and 3 d of RIT. At each time point, cells were rinsed and incubated in RIPA buffer (Santa Cruz) at 4° C. for 30 minutes. Cells were centrifuged and supernatant (containing proteins) were collected. Proteins were electrophoresed through SDS-PAGE using 12% poly-acrylamide gels and electrotransferred onto nitrocellulose membranes. Blots were incubated with anti-CDK1, anti-p-CDK1(Tyr15) (clone 10A11), anti-p-CDK1(Thr14), anti-p-CDK1(Thr161), anti-CDK7, anti-Wee-1 (clone D10D2), anti-Myt-1, anti-p-CHK1 (Ser345) and anti-human GAPDH (1/1000, Cell Signaling Technologies, Leiden, The Netherlands) primary antibodies. Blot were washed and incubated with horseradish peroxidase-conjugated anti-mouse Ig (115-036-072, Jackson ImmunoResearch) or with horseradish peroxidase-conjugated anti-rabbit Ig (7074, Cell Signaling). Signal detection of immunoblots was carried out using the enhanced chemiluminescence protocol according to the manufacturer's instructions (Clarity™ Western ECL blotting substrates, 1705061, BioRad). PXi analyser (Ozyme) was used to measure levels of protein expression.

### 4. Wee-1 and Myt-1 Inhibitors

Cells were treated for 18 h with 1 μM of the selective Wee-1 kinase inhibitor MK-1775 (Selleckchem, Houston, USA) or of the dual Wee-1/Myt-1 inhibitor PD-166285 (EMD Merck Millipore/Calbiochem, Molsheim, France) alone or in combination with 6 MBq/mL, 2 MBq/mL and 0.5 MB/mL of ¹⁷⁷Lu-lilotomab for Ramos, Rec-1 and DOHH2 cells respectively. At different times after start of incubation, proteins were extracted to measure the CDK1 phosphorylation levels, in parallel, proliferation was determined and at 18 h and 24 h after start of incubation, the percentage of cells in G2/M was assessed (experiments were performed three time except the cell cycle analysis).

### 5. Expression of Stem Cell Markers: CD44 and CD33

Stem cell markers (CD133 and CD44) were assessed in 1×10⁶ Ramos, DOHH2 or Rec-1 cells/mL grown in 12-well plates and exposed for 18 h to 0 and 6 MBq/mL of ¹⁷⁷Lu-lilotomab, ¹⁷⁷Lu-rituximab or ¹⁷⁷Lu-cetuximab. Cells were harvested at 0, 2 h, 18 h and each day from 1 d to 10 d of RIT. At each time point, cells were fixed with PFA 4% for 10 minutes, washed twice with PBS and sterilely stored in PBS at 4° C. Stem cell markers were detected with anti-CD133-FITC (clone: AC133, Milteniy) and anti-CD44-APC (clone: IM7, Merck Millipore). 0.5×106 cells were saturated for 1 h in PBS-BSA 0.5%. Next cells were centrifuged and incubated with anti-CD133-FITC (2 μL) and anti-CD44-APC (0.125 μg) for 1 h at room temperature in the dark. Finally, cells were rinsed twice and analysed using Gallios® flow cytometer (Beckman Coulter).

### E. Cellular Dosimetry

### 1. Cellular Uptake of Radioactivity and Cells Numbering

As a preliminary step towards cellular dosimetry, the uptake of radioactivity was determined in Ramos, DOHH2 and Rec-1 cells exposed for 18 h either to ¹⁷⁷Lu-lilotomab, ¹⁷⁷Lu-rituximab or ¹⁷⁷Lu-cetuximab. Typically, 1×10⁶ Ramos, DOHH2 and Rec-1 cells/mL of culture medium were incubated for 18 h with increasing activities (0; 0.5; 1; 2; 4 and 6 MBq/mL) of ¹⁷⁷Lu-labelled mAbs. Then, cells were washed twice with culture medium and seeded in 12 micro-well plates containing 1 mL of culture medium at a concentration of 200×10³ cells/mL. At various times, (2 h, 18 h, 24 h, 48 h, 72 h and 144 h), cells were collected, washed twice with PBS, resuspended in 1 ml of PBS and gamma counted (Hewlett Packard, Palo Alto, Calif.). An aliquot fraction (8 μL) was used for cells numbering using cell counter (Muse, Merck Millipore). The activity per cell (Bq/cell) was calculated and plotted as a graph expressing the uptake of radioactivity per cell (Bq/cell) as a function of time (hours). For all cell lines and each mAb, experiments were done in triplicates and repeated at least three times.

### 2. Cell Geometry and Behaviour in Flask Culture

The cell and nucleus dimensions of Ramos cells were determined after propidium iodide staining and fluorescent microscopic analysis of Ramos cells. For both cell and nucleus, the area and the size corresponding to the largest and smallest diameters were determined. For DOHH2 and Rec-1 cells, the cell dimension were determined with Scepter™ 2.0 Cell Counter (Merck) and the nucleus dimensions were determined after Dapi staining and fluorescent microscopic analysis.

During incubation with the radiopharmaceuticals the cells showed the tendency to accumulate at the centre of the culture, and to form clusters of different sizes. Since the density of cells (isolated), and clusters was very heterogeneous within the culture well, a preliminary determination of these parameters was performed on the basis of pictures acquired by optical microscopy. Four sets of pictures were acquired, corresponding to Ramos and DOHH2 cells treated with ¹⁷⁷Lu-rituximab and ¹⁷⁷Lu-lilotomab. Three regions were identified in the culture well: centre, halfway and edge. For each region, and for each of the four cell/antibody combinations, two pictures were taken at ×50 and ×200 magnifications, in order to measure the cell density in each area.

### III. In Vivo Studies

### A. Determination of Maximal Tolerated Activity (MTA)

a. In Athymic Nude Mice

Mice were intravenously injected with 100 μL of ¹⁷⁷Lu-lilotomab, ¹⁷⁷Lu-rituximab or ¹⁷⁷Lu-cetuximab in NaCl. Three different activities were assessed: 400, 500 or 600 MBq/kg (n=4 for each test activity and for each mAb) and additionally 300 and 350 MBq/kg for ¹⁷⁷Lu-rituximab. Then mice were weighed every three days to evaluate the potential toxicity of the treatment (end points were weight loss higher than 20% or any signs of sickness or discomfort).

b. In Scid Mice

One day before treatment with ¹⁷⁷Lu-mAbs, mice were intraperitoneally injected with 10 mg/kg of murine unspecific IgG2a (M7769, Sigma-Aldrich, Saint-Louis USA). Then, mice were intravenously injected with 100 μL of ¹⁷⁷Lu-lilotomab, ¹⁷⁷Lu-rituximab or ¹⁷⁷Lu-cetuximab in NaCl. Three different activities were assessed, 75, 125 or 150 MBq/kg (n=4 for each test activity and for each monoclonal antibody). Finally, mice were weighed every three days to evaluate the potential toxicity of the treatment (end points were weight loss higher than 20% or any signs of sickness or discomfort).

### B. Survival Experiments

a. In Athymic Nude Mice

Mice were subcutaneously xenografted with 10×10⁶ Ramos NHL cells resuspended in 100 μL of fresh serum-free medium in the right flank. Mice were treated 13 days post xenograft when tumour volume reaches 100-200 mm³. In control groups, mice (n=8/treatment group) were intravenously injected with 100 μL of NaCl, lilotomab or rituximab mAb at the same quantity used during the RIT and at high concentration (10 mg/kg). For RIT experiments, mice (n=6-9/treatment group) were intravenously injected (100 μL) with MTA of either ¹⁷⁷Lu-lilotomab, ¹⁷⁷Lu-rituximab or ¹⁷⁷Lu-cetuximab mAb. Tumour growth was evaluated by measuring the tumour volume with a calliper (a, b, c in the formula below represent the three diameters) and animal weight was determined two or three times per week.

The tumour volume was calculated using the following equation:

$$Tumour volume = \frac{( a \times b \times c )}{2}$$

Mice were sacrificed using CO₂ asphyxiation or cervical dislocation, when the tumour volume reaches 2000 mm³ or when the weight loss was higher than 20% or signs of sickness or discomfort.

b. In Scid Mice

Mice were subcutaneously xenografted with 10×10⁶ DOHH2 NHL cells in 100 μL of fresh serum-free medium in the right flank. One day before treatment (D+6 post-xenograft), mice were intraperitoneally injected with 10 mg/kg of unspecific IgG2a (M7769, Sigma-Aldrich, Saint-Louis USA). Mice were treated 7 days post xenograft (n=7/treatment group), in control groups, mice were intravenously injected with 100 μL of NaCl, lilotomab or rituximab mAb at the same quantity used in the RIT experiment and at high concentration (10 mg/kg). For RIT experiments, mice were intravenously injected (100 μL) with MTA of ¹⁷⁷Lu-lilotomab, ¹⁷⁷Lu-rituximab or ¹⁷⁷Lu-cetuximab. Tumour growth was assessed by measuring the tumour volume using a calliper (a, b, c in the formula above represent the three diameters) and animal weight was determined two or three times a week. The tumour volume was calculated using the previous equation. Mice were sacrificed using CO₂ asphyxiation, when the tumour volume reached 2000 mm³ or when the weight loss was higher than 20% or signs of sickness or discomfort.

c. Haematological Toxicity

Blood samples (about 12 μL) were collected from tail vein twice a week the first month post-treatment. They were analysed using the Scil Vet abc system (SCIL Animal Care Co., Altorf) to determine haematological toxicity.

### C. In Vivo Dosimetry

### 1. Biodistribution Experiments

### a) In Athymic Nude Mice

Mice were subcutaneously xenografted with 10 million of Ramos cells as in therapeutic experiment. 13 days later, mice were intravenously injected with therapeutic experiment used in survival experiment. Two protocols were then performed.
First protocol: 25 mice were treated with ¹⁷⁷Lu-lilotomab. At each time point post-treatment (1 h, 1 d, 2 d, 3 d and 6 d), SPECT-CT images were performed on five mice and these mice were necropsied. Each organs were collected, weighed and their uptake of radioactivity were measured through γ-counting. This protocol allows the comparison between the ex vivo counting and the in vivo image-based biodistributions.
Second protocol: mice were intravenously injected with ¹⁷⁷Lu-rituximab or ¹⁷⁷Lu-cetuximab (5 mice per treatment). At different times post-treatment (1 h, 1 d, 2 d, 3 d and 6 d), each mouse were SPECT-CT imaged and at the last time point, mouse were necropsied. This protocol was to perform an in vivo Biodistribution (to reduce necropsied mice number).
   a. In Scid Mice

75 mice were subcutaneously xenografted with 10 million of DOHH2 cells. 6 days later, mice were intraperitoneally injected with 10 mg/kg of IgG2a (M7769, Sigma-Aldrich, Saint-Louis USA). Next day, mice were intravenously injected with the therapeutic activities used in survival experiment of ¹⁷⁷Lu-lilotomab, ¹⁷⁷Lu-rituximab or ¹⁷⁷Lu-cetuximab (25 mice per radiolabelled mAb). Then, at different times post-treatment (1 d, 2 d, 3 d and 6 d), mice were sacrificed by lethal injection (2.5 mL/kg) of ketamine (26 mg/mL)/medetomidine (0.30 mg/mL) (n=5 mice/time of dissection). Finally, organs were collected, weighed and their uptake of radioactivity were measured.

For each organ, percentage of injected activity per gram of tissue as a function of time were plotted (corrected decays).

### 2. S-Factor Determination

S-factors S(t→s) correspond to the average absorbed dose in a target region t per radioactive disintegration in a source region s. S-factor takes into account contributions of all types of emitted particles by the source and composition of the matter. S-values used in this project were obtained by Monte-Carlo simulations in the MOBY voxelised phantom with ¹⁷⁷Lu.

### IV. Statistical Analysis

The statistical analysis was performed by the Department of statistics of ICM Val d'Aurelle, Montpellier. Data were described using median, mean and standard deviation. A linear mixed-regression model was used to determine the relationship between tumour growth and number of days after graft in in vivo experiments. Survival rates were estimated from the xenograft date to the date of the event of interest (i.e., a tumour volume of 2000 mm³) using the Kaplan-Meier method. The log-rank test was used to compare survival curves between groups. In vitro data (cell cycle and inhibitor effects) were compared with the non-parametric Kruskal-Wallis test. The significance level was set at p<0.05. Statistical analyses were performed using the Stata software v13.0 (Stata Corporation College Station, USA).

### Results & Discussion

This aimed at investigating in vitro the molecular mechanisms involved in the cytotoxic effects of ¹⁷⁷Lu-lilotomab and ¹⁷⁷Lu-rituximab in Ramos, DOHH2 and Rec-1 cells.

Cycle progression, apoptosis induction, expression of cell signalling proteins, and stem cells marker expression have been investigated in cells exposed to ¹⁷⁷Lu-mAbs. ¹⁷⁷Lu-cetuximab was used as a non-specific antibody, i.e. a control of nonspecific irradiation.

### A. Apoptosis Induction

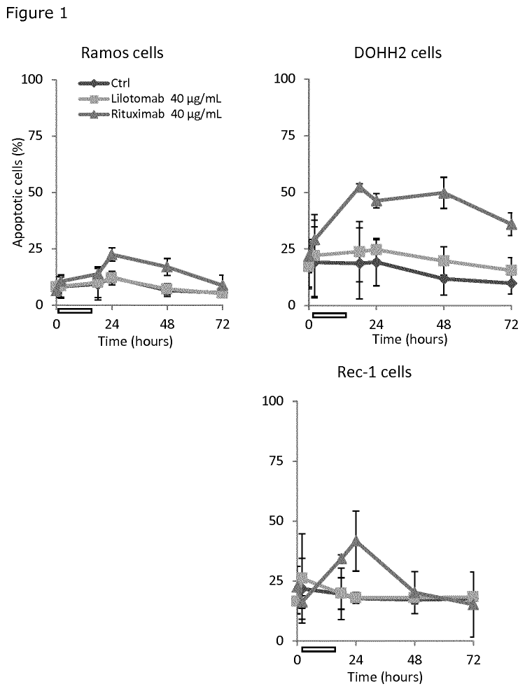

Since the role of apoptosis has extensively been highlighted in radiation-induced cell death of haematological cells and during cell response to rituximab, we measured apoptosis in Ramos, DOHH2 and Rec-1 cells following incubation with unlabelled or radiolabelled mAbs. Incubation of DOHH2 with rituximab was accompanied by a strong apoptosis induction with a plateau at 18 h (52±1%) lasting until day 2. Apoptosis level was lower in Ramos cells (22±3%) at peak time of 24 h), and intermediate for Rec-1 cells (42±12%) at peak time of 24 h). No apoptosis was induced following treatment with lilotomab (FIG. 1).

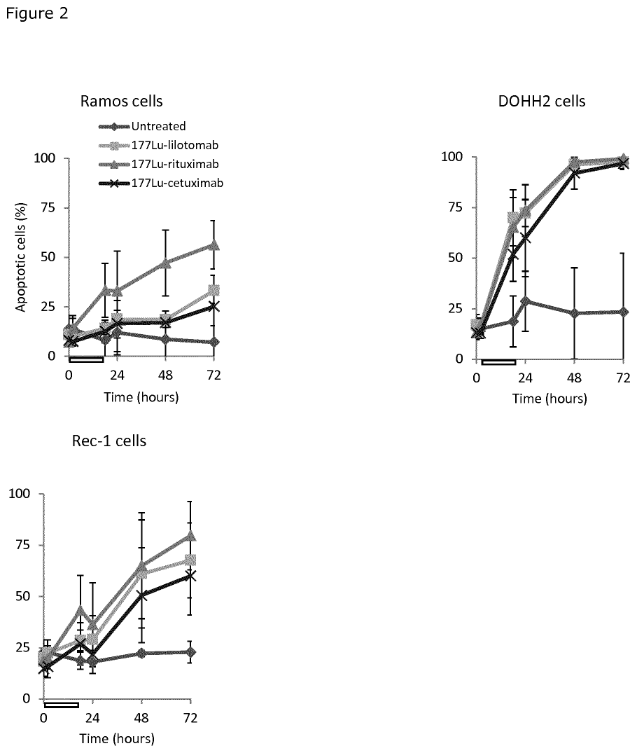

Apoptosis was induced in the three cell lines (FIG. 2) treated with radiolabelled mAbs. After exposure to ¹⁷⁷Lu-rituximab, the highest level of apoptosis was measured in DOHH2 cells (97±3% at peak time of 72 h) while the lowest was observed in Ramos cells (56±12%, at peak time of 72 h), and an intermediate one in Rec-1 cells (79±16%, at peak time of 72 h). In DOHH2 cells, a similar trend was observed for the three ¹⁷⁷Lu-mAbs. In Ramos cells, apoptosis reached a maximal level at 72 h with similar levels after exposure to ¹⁷⁷Lu-lilotomab and ¹⁷⁷Lu-cetuximab (33±8% and 27±10%, respectively). In Rec-1 cells, a peak of apoptosis was measured at 72 h with values of 79±16%, 67±18% and 60±19% following exposure to ¹⁷⁷Lu-rituximab, ¹⁷⁷Lu-lilotomab and ¹⁷⁷Lu-cetuximab, respectively.

### B. Cell Cycle Effect in Ramos, DOHH2 and Rec-1 Cell Lines

The distribution of Ramos, DOHH2 and Rec-1 cells through cell cycle following exposure to 0, 2 (data not shown) and 6 MBq/mL of ¹⁷⁷Lu-mAbs was investigated.

1. Following Exposure to Radiolabelled mAbs

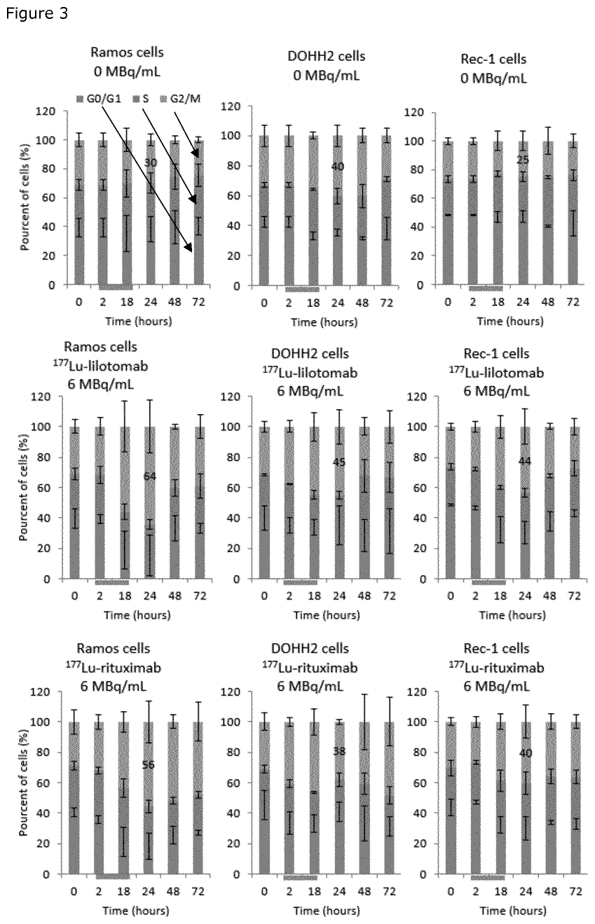

FIG. 3 shows that for non-exposed Ramos, DOHH2 and Rec-1 cells, the distribution in cell cycle phases was: 39-48% in G0/G1, 25-35% in S, and 23-40% in G2/M. When cells were exposed to 6 MBq/mL of ¹⁷⁷Lu-lilotomab, the proportion of cells showing a G2/M cell cycle arrest at 24 h was 64±16%, 45±11% and 44±11% for Ramos, DOHH2 and Rec-1 cells, respectively. Corresponding values in untreated cells were 30%, 30% and 25%, respectively. Moreover, when cells were exposed to 6 MBq/mL of ¹⁷⁷Lu-rituximab, the proportion of cells showing a G2/M cell cycle arrest at 24 h was 56±13%, 38±2% and 40±11% for Ramos, DOHH2 and Rec-1 cells, respectively.

2. Following Exposure to Unlabelled mAbs

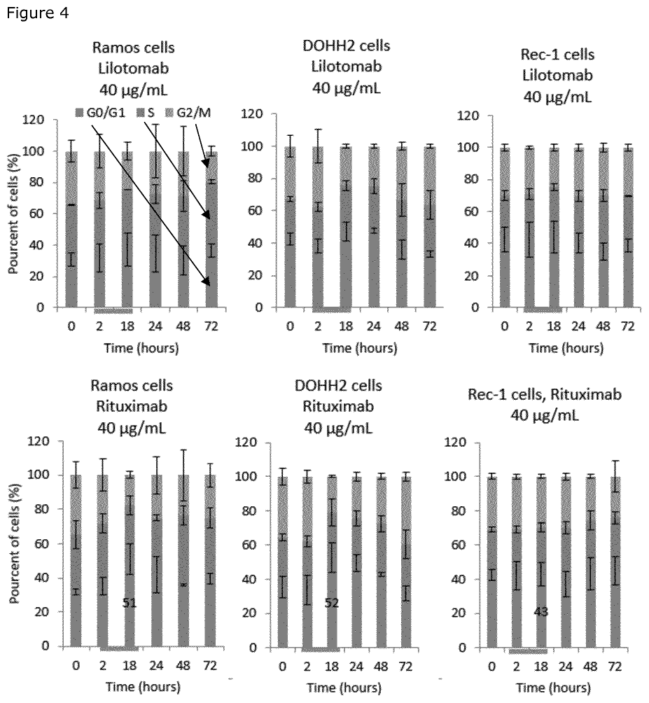

When the three cell lines were exposed to 40 μg/mL lilotomab (FIG. 4), the distribution of cells in the cycle phases was similar to the one of untreated cells. Moreover, when cells were exposed to 40 μg/mL rituximab, the proportion of cells showing a G1/G0 cell cycle arrest at 18 h was 51±9%, 52±9% and 43±7% for Ramos, DOHH2 and Rec-1 cells, respectively. Corresponding values for untreated cells were 31±2%, 35±6% and 42±3%, respectively.

### 3. Key Points

The radiobiological response of cells to mAbs or ¹⁷⁷Lu-mAbs treatment was assessed:
Apoptosis was induced after treatment with rituximab but not after lilotomab treatment in the three cell lines.
¹⁷⁷Lu-mAbs induced apoptosis in the three cell lines with a higher level in the radiosensitive DOHH2 cell line and a lower level in the radioresistant Ramos cell line.
¹⁷⁷Lu-lilotomab induced as much apoptosis as ¹⁷⁷Lu-rituximab in the radiosensitive DOHH2 cell line whereas in the two other cell lines, the induction was lower.
¹⁷⁷Lu-mAbs led to an increase of the number of Ramos cells in G2/M compared to untreated cells (×2), whereas this increase did not occur in the radiosensitive DOHH2 cell line (×1.1) (Rec-1 was between both; ×1.7).
A cell cycle arrest in G1 phase was observed after treatment with rituximab in Ramos (×1.6) and DOHH2 (×1.5) but not in Rec-1 cell line.

Apoptosis induction was inversely proportional to G2/M cell cycle arrest. Indeed, Ramos cell line being the most radioresistant model, showed a weak induction of apoptosis after treatment with ¹⁷⁷Lu-mAbs but displayed the highest accumulation of cells in G2/M. Conversely, DOHH2 cell line which was the most radiosensitive model, showed the highest level of induction of apoptosis after treatment with ¹⁷⁷Lu-mAbs but also demonstrated the lowest arrest in G2/M. We hypothesised that the G2/M cell cycle arrest was a major component of the cell radiosensitivity. The proteins involved in this arrest were investigated.

### C. Proteins Involved in G2/M Cell Accumulation

The CDK1 kinase is a major protein involved in the control of the G2/M transition. This kinase is tightly regulated by the inhibitory phosphorylations on its Thr14 and Tyr15 residues respectively by the Myt-1 and Wee-1 kinases and by the activating phosphorylation on Thr161 by the CDK-activating kinase.

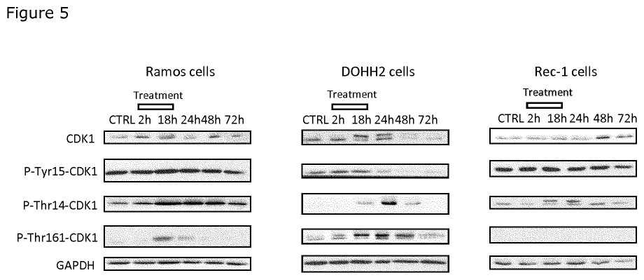

The expression of CDK1 kinase and its phosphorylations were investigated (FIG. 5).

CDK1 level was quite stable in Ramos and Rec-1 cells, whereas it decreased in DOHH2 at 48 h and 72 h after ¹⁷⁷Lu-lilotomab addition. In Ramos cells high persistent levels of the inhibitory pTyr15 and pThr14 CDK1 phosphorylations were present whereas transient low levels of the activating pThr161 phosphorylation were observed at 18-24 h after exposure to ¹⁷⁷Lu-lilotomab. Similar results were shown with the Rec-1 cells, however, pThr161-CDK1 was undetectable. Conversely, the opposite was observed in DOHH2 cells. Phosphorylation levels of Tyr15 and Thr14 dropped respectively at 24 h and 48 h after ¹⁷⁷Lu-lilotomab exposure. This decrease in levels of inhibitory phosphorylations was associated with a persistent increase in the expression of the activating pThr161 phosphorylation.

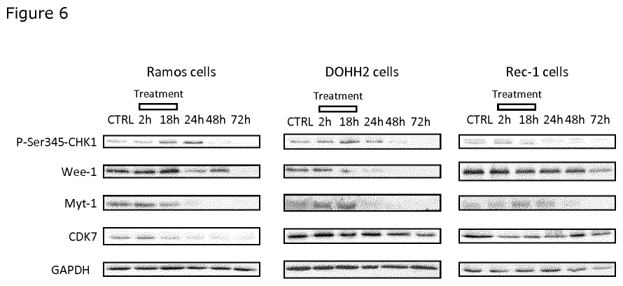

The levels of CDK7, which is part of the complex involved in the phosphorylation of CDK1 on its Thr161 residue, was stable or increased in DOHH2 and Rec-1 compared with Ramos cells (FIG. 6). Also, expression of Wee-1, which mediates CDK1 phosphorylation at Tyr15 leading to G2/M arrest, was comparable in control and treated Ramos and Rec-1 cells between 18 and 48 h. Conversely, in DOHH2 cells, baseline was low and became undetectable at 24 h post-treatment. These observations supported our previous results on the accumulation of Ramos, but not DOHH2 cells, in G2/M cell cycle phase following exposure to ¹⁷⁷Lu-lilotomab. The level of phosphorylated CHK1 (Ser345), which is involved in Wee-1 and Myt-1 activation after cell irradiation, was increased in Ramos and DOHH2 cells at 18-24 h, but not in Rec-1 cells where it remained stable until 18 h before progressively decreasing. Expression of Myt-1, the kinase involved in Thr14-CDK1 phosphorylation that also contributes to a G2/M arrest, decreased in all three cell lines 18 h after treatment.

### D. Wee-1 and Myt-1 Inhibitors

Since Wee-1 and Myt-1 kinases seemed to be involved in the G2/M cell cycle phase accumulation in Ramos cells exposed to ¹⁷⁷Lu-lilotomab, cells were treated with the MK-1775 and PD-166285 pharmacological inhibitors of theses kinases in combination with ¹⁷⁷Lu-lilotomab. MK-1775 inhibits the Wee-1 catalytic activity and subsequently the pTyr15-CDK1 phosphorylation. PD-166285 inhibits both Wee-1 and Myt-1 and subsequently the phosphorylations of CDK1 on both Tyr15 and Thr14.

### 1. Proliferation Study

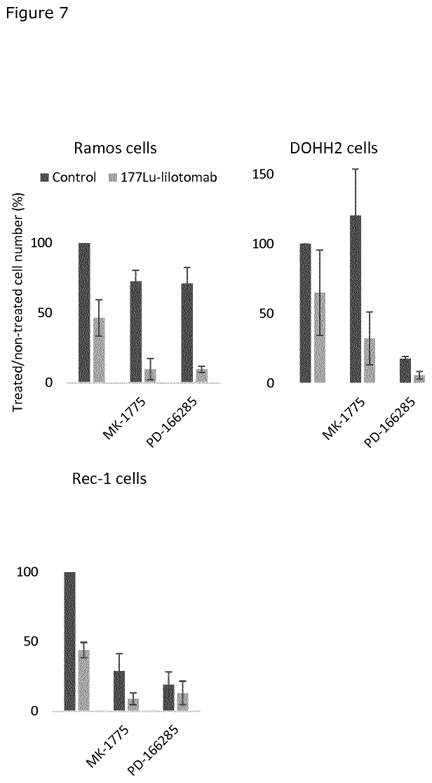

FIG. 7 shows the ratio between the number of cells exposed for 18 h to ¹⁷⁷Lu-lilotomab or to the inhibitor alone or to the combination and the number of untreated cells, 6 days after start of treatment. The activity used for the treatment with ¹⁷⁷Lu-lilotomab was chosen to decrease the proliferation to about 50% for the three cell lines compared with non-treated cells.

In Ramos cells, both inhibitors induced a decrease of the proliferation to 70% and the combinations of RIT with MK-1775 or RIT with PD-166285 induced a similar decrease close to 10%. Those decreases in proliferation were statistically different from treatments using ¹⁷⁷Lu-lilotomab or the inhibitors alone (p=0.0495) but no difference was observed between the two combinations (p=0.5127)

For DOHH2 cells, only the PD-166285 was shown to modify proliferation with a strong decrease (18%). Although the anti-proliferative effect of ¹⁷⁷Lu-lilotomab was more pronounced in the presence of the two inhibitors, no statistical difference was shown compared with the inhibitors alone (p=0.1213) or with ¹⁷⁷Lu-lilotomab alone (p=0.1213).

A significant reduction in proliferation was also observed for Rec-1 cells exposed to inhibitors (p=0.0495). However, only the combination of ¹⁷⁷Lu-lilotomab and MK-1775 statistically reduced the proliferation compared with the inhibitor alone (p=0.0495) or to the ¹⁷⁷Lu-lilotomab alone (p=0.0495).

### 2. Phosphorylation of CDK1

The therapeutic efficacy of both combinations (RIT+MK1775 or RIT+PD166285) corroborated the phosphorylation of the CDK1 in the three cell lines.

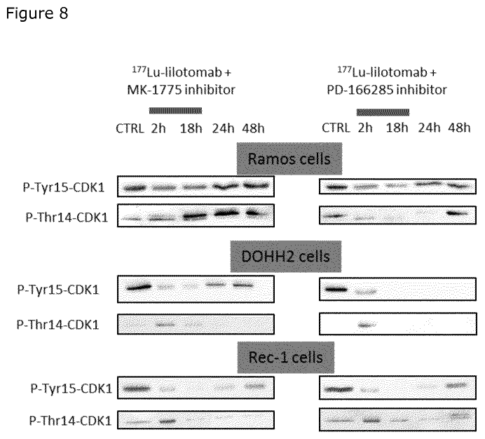

The relatively high persistent levels of pTyr15-CDK1 and pThr14-CDK1 in Ramos cells exposed to ¹⁷⁷Lu-lilotomab were then decreased in the presence of the corresponding inhibitors at 2 and 18 h before re-increasing (FIG. 8). In DOHH2 cells, this re-increase after 18 h was less pronounced or even not observable for pTyr15-CDK1; for pThr14-CDK1, the basal level was too low to be modulated and detected. For Rec-1 cells, since basal level of pThr14-CDK1 was low, inhibitors did not show any effect on phosphorylation expression whereas expression of pTyr15-CDK1 was decreased during treatment and increased at 48 h.

### 3. Inhibition of G2/M Cell Cycle Arrest

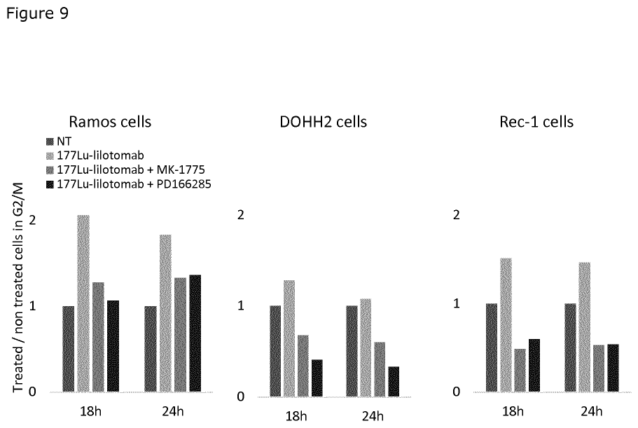

FIG. 9 shows the variations between the number of cells in G2/M after treatment with ¹⁷⁷Lu-lilotomab alone or combined with MK-1775 or PD-166285, and the untreated cells. As previously shown, the proportion of Ramos and Rec-1 cell in G2/M phase was increased following treatment with ¹⁷⁷Lu-lilotomab. When the inhibitors were combined with ¹⁷⁷Lu-lilotomab, the proportion of cell in G2/M decreased. In Ramos cells, after treatment with both combinations, the proportion of G2/M cells became similar to that of untreated cells. For Rec-1 cells, both combinations decreased by half the proportion of cells in G2/M. In DOHH2 cells, the decrease in the proportion of G2/M cells induced by the combination ¹⁷⁷Lu-lilotomab and PD-166285 is higher than that induced by the combination with the MK-1775.

### 4. Key Points

Both combinations showed a strong decrease in proliferation of Ramos cells that was associated with a reduction of CDK1 phosphorylations leading to a decrease in G2/M cells proportion. No difference in proliferation of cells treated with the combinations 177Lu-lilotomab and MK-1775 or ¹⁷⁷Lu-lilotomab and PD-166285 was observed, showing that the principal phosphorylation playing a role in the G2/M cell cycle arrest was the pTyr15-CDK1 in the Ramos cell line.

In DOHH2 cells, the basal level of the two inhibitory phosphorylations (p14 and p15) was weak. When the inhibitors were used, cell proliferation was decreased (particularly for the PD-166285) but this was not statistically significant and was in lower extent than in Ramos cell line.

For Rec-1 cell line, the major inhibitory phosphorylation was the P-Tyr15-CDK1. When the inhibitors were associated with ¹⁷⁷Lu-lilotomab, the MK-1775 was as efficient as the PD-166285 in reducing cell proliferation corroborating the importance of the P-Tyr15-CDK1 in Rec-1 cell response.

### E. Discussion

In this part we investigated the biological mechanisms that could explain why ¹⁷⁷Lu lilotomab is more efficient in DOHH2 cells than in Ramos cells, Rec-1 cells showing intermediary response. Moreover, we showed in part I that synergy between radiation and rituximab was observed, although at different extent, in both Ramos and DOHH2 cells while synergy was only observed in DOHH2 cells for ¹⁷⁷Lu-lilotomab. The latter results were supported by in vivo data where ¹⁷⁷Lu-lilotomab was as efficient as ¹⁷⁷Lu-rituximab in DOHH2 tumour xenograft model, although unlabelled rituximab was more efficient than lilotomab.

Apoptosis Induction is Higher in Radiosensitive DOHH2 Model after Treatment with ¹⁷⁷Lu-mAbs

Since haematological disease is known to respond to irradiation through apoptosis, apoptosis induction was measured in Ramos, DOHH2 and Rec-1 cells after treatment with unlabelled or radiolabelled mAbs. In agreement with previous studies, rituximab was shown to induce strong apoptosis induction in the three cell lines whereas lilotomab not. However, when mAbs were radiolabelled, apoptosis level was increased in the three cell lines but mostly for DOHH2 cells in a similar way for both ¹⁷⁷Lu-lilotomab and ¹⁷⁷Lu-rituximab. Apoptosis level was lower in Ramos cells and in between for Rec-1 cells. These results are in agreement with in vitro and in vivo results indicating that the therapeutic efficacy of ¹⁷⁷Lu-lilotomab and ¹⁷⁷Lu-rituximab is higher in DOHH2 models and can be correlated with observed cell radiosensitivity.

Radioresistant Ramos Model is Characterised by an Increase Number of Cells in G2/M after Treatment with 177Lu-mAbs

Since apoptosis is tightly under the control of cell cycle checkpoints, the distribution of treated cells through cell cycle phases (G0/G1, S and G2/M) was analysed. In response to ¹⁷⁷Lu-mAb treatments, the number of cells in G2/M was strongly increased compared to untreated cells in the radioresistant cell line while it was not in the most radiosensitive cell line. During G2 phase, CDK1 is activated by binding to cyclins A2 and B. When entering M phase, cyclin A2 is degraded and the CDK1-cyclin B complex remains that will be further degraded during late mitosis. Besides the association of CDK1 with cyclins, G2/M cell cycle progression is promoted when CDK1 is phosphorylated on Thr161. Conversely, a phosphorylation on Tyr 15 by Wee-1 and/or on Thr14 by Myt-1 blocks the cells in G2/M. In the radiosensitive DOHH2 cells, after treatment with ¹⁷⁷Lu-lilotomab, pTyr15-CDK1 and pThr14-CDK1 levels are decreased, whereas pThr161-CDK1 ones are increased. Conversely in Ramos and Rec-cells, the expression of pTyr15-CDK1 and pThr14-CDK1 is high whereas the expression of pThr161-CDK1 is low. These proteins phosphorylation are in agreement with cell cycle analysis. Then, G2/M arrest would be the major checkpoint affecting the radiosensitivity of the cell lines. G2/M arrest provides cells time to repair DNA damage in response to ¹⁷⁷Lu-mAb treatment, before progressing through cell cycle. In DOHH2 cells, lack of G2/M arrest is accompanied by strong apoptosis induction. In order to confirm the role of G2/M arrest, inhibitors of the phosphorylation of the CDK1 on Thr14 and Tyr15 were used. The MK-1775, a specific inhibitor of Wee-1 and PD-166285 inhibiting both Wee-1 and Myt-1 were used. These inhibitors were used in combination with ¹⁷⁷Lu-lilotomab. First, inhibition of the Wee-1 and Myt-1 kinase activity was confirmed by Western Blotting since a decrease in CDK1 phosphorylations was observed in the three treated cell lines. Subsequently, the percentage of cells in G2/M was shown to decrease in cells treated with the combinations compared to the cells only treated with ¹⁷⁷Lu-lilotomab in all the cell lines. Next, the anti-proliferative effects of these inhibitors on cells exposed to ¹⁷⁷Lu-lilotomab was shown with a more marked effect in the radioresistant Ramos cell line. The results confirm the implication of the CDK1 phosphorylations in cell response after treatment with radiolabelled mAbs.

Inhibition of G2/M cell cycle arrest radiosensitises radioresistant Ramos model. The Bliss independence mathematical model was next used to investigate the role of MK-1775 or PD-166285 on the response to ¹⁷⁷Lu-lilotomab. A theoretical therapeutic efficacy was calculated for the two combinations (MK-1775 or PD-166285) in the three cell lines. And the comparison between the experimental and the theoretical curves was done.

Theoretical efficacy_{RIT+inhib}=Efficacy_{RIT}+Efficacy_{inhib}−(Efficacy_{RIT}×Efficacy_{inhib})

A synergy (p=0.0495) between inhibitors and ¹⁷⁷Lu-lilotomab was shown in Ramos cells. In DOHH2 and Rec-1 cells, the combination was shown to be additive only. This can probably be explained by the fact that G2/M cell cycle arrest is less marked in Rec-1 cells and absent in DOHH2 cells.

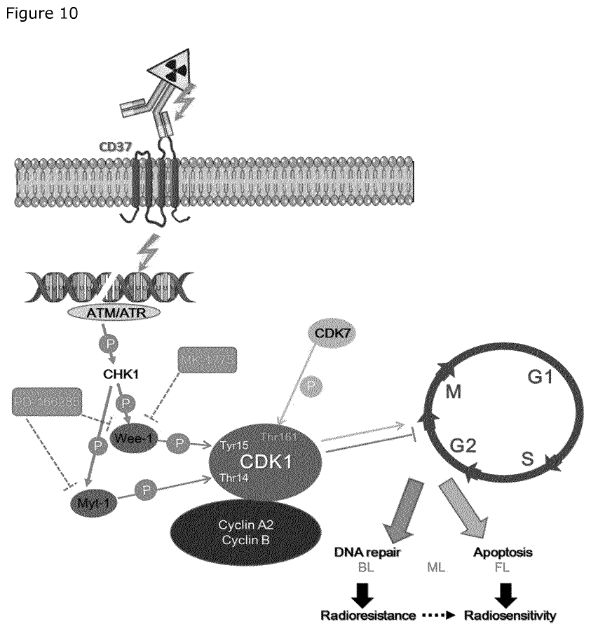

Finally, the mechanism of action of ¹⁷⁷Lu-lilotomab described in FIG. 10 was proposed.

### Conclusion & Perspectives

This project aimed at investigating the molecular mechanisms underlying tumour cell response.

¹⁷⁷Lu-lilotomab was more efficient than rituximab in transformed follicular lymphoma preclinical models. ¹⁷⁷Lu-lilotomab was also efficient in Burkitt's lymphoma cells, but much higher doses were required. Moreover, reduced CDK1-mediated G2/M cell cycle arrest was shown to predict ¹⁷⁷Lu-lilotomab efficacy. Release of Ramos and Rec-1 cells from G2/M cell cycle arrest using a Wee-1 pharmacological inhibitor (MK-1775) sensitised these cells to ¹⁷⁷Lu-lilotomab. These results support clinical studies showing that ¹⁷⁷Lu-lilotomab was particularly active in relapsed indolent lymphoma. Finally, it must be noted that in our experimental approach, immunological response was reduced because we used immunodeficient mice, although some ADCC effects could be expected because NK cells are active in both mouse strains. In a clinical setting, the immunological response could be enhanced by using the chimeric version of lilotomab that can activate ADCC.

Presence of Stem Cell Markers on Radiosensitive DOHH2 Cells after Treatment

A preliminary study to determine how cells can survive after treatment was performed. Cancer stem cells are described by the American Association of Cancer Research, as cells that “constitute a reservoir of self-sustaining cells with the exclusive ability to self-renew and maintain the tumour”. To better understand why the radiolabelled mAb treatment did not eradicate the tumour cells in the petri dish even in the radiosensitive cell line, the expression of the cancer stem cell markers at the surface of the treated cells was analysed from the beginning of the treatment to 9 days post-treatment.

A variety of cancer stem cells have been identified in an increasing number of epithelial tumours, including breast, prostate, pancreatic, and head and neck carcinomas, the majority of them express the cell-surface glycoprotein CD44. Another cell surface marker, the CD133 glycoprotein, defined the tumour-initiating cells of brain and colon carcinomas. In lymphoma, a first study could indicate the existence of the CD45+/CD19− subpopulation in Mantle lymphoma which are highly tumorigenic and display self-renewal capacity in vivo.

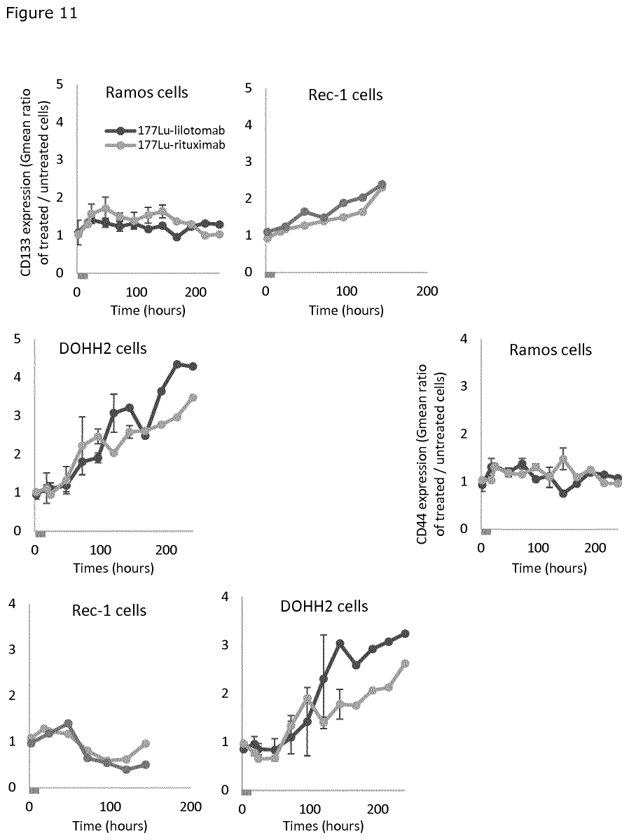

For a preliminary study, investigation of the expression of CD133 and CD44 at the surface of cells treated for 18 h with 6 MBq/mL ¹⁷⁷Lu-lilotomab and ¹⁷⁷Lu-rituximab was done. FIG. 11 shows the ratio between the number of receptors at the treated cell surface and the number of receptors at the untreated cell surface.

The proportion of cells expressing CD44 and CD133 strongly increased up to 9 days post-RIT in the radiosensitive DOHH2 cell line exposed to ¹⁷⁷Lu-lilotomab or ¹⁷⁷Lu-rituximab and also in Rec-1 cells to a lower extent, but not in Ramos cells. These results suggest that after RIT in the radiosensitive cell line, the population of surviving cells is modified. The hypothesis is that this population is resistant to the treatment and allows forming afresh the tumour; in the radioresistant cell line, the treatment did not select enough stem cell population to show a modification of the cell population.

In order to go further, it would be interesting to determine if this new population (CD133+/CD44+) is more radioresistant than the primary population and to determine the differences of characteristics between this new population and the primary one (time of culture growth, xenograft growth, response to treatment, . . . ). Finally, another question is to know if the population becomes CD133+/CD44+, or if the treatment selects the cells CD133+/CD44+ already present.

### Combination PIT and Wee-1 Inhibitor in the Clinic

Anyway, it would be interesting to consider the combination (RIT+ cell cycle arrest inhibitor) for clinical treatment. Indeed, in all the cell lines the combination is always more efficient on cell proliferation than RIT alone. In clinic, we could think that the results will be similar. Even, without data on the tumour radiosensibility:
if the tumour is radioresistant, the combination of ¹⁷⁷Lu-lilotomab and the cell cycle arrest inhibitor potentiates the effect of ¹⁷⁷Lu-lilotomab and radiosensitises the tumour;
if the tumour is radiosensitive, the combination is also more effective than the RIT alone, at least due to the additivity of the effects, but in a lower extent than in the previous case.

Furthermore, this association is especially interesting, as inhibitors of proteins required for cell cycle progression, such as CD4/CDK6, pan-CDK and Wee-1 inhibitors, have gained interest for cancer therapy of haematological tumours and are assessed in clinical trials. Particularly, MK-1775 enhances the efficacy of SRC inhibitors in Burkitt's lymphoma and the combination of CHK1 and Wee-1 inhibitors is synergistic in mantle cell lymphoma.

Moreover, clinical trials are assessing CHK1 pharmacological inhibitors to sensitize tumour cells to DNA damage, because CHK1 is involved in Wee-1 and Myt-1 phosphorylations. However, it must be noted that in our experimental model, P-CHK1 expression were not modified by exposure to ¹⁷⁷Lu-mAbs.

### Implication of the Protein 14-3-3 in Cell Radiosensitivity?

Other proteins could be targeted to potentiate the effect of ¹⁷⁷Lu-lilotomab. For example, the protein 14-3-3 can also be a great candidate of targeting during a RIT. A critical role of 14-3-3 proteins in cancer has been studied particularly in breast, lung and head and neck cancers. In support of a predominant role of 14-3-3, high expression of 14-3-3 is associated with poor prognosis of breast cancer patients.

This protein is implicated in the cytoplasmic sequestration of CDC25C and prevents mitotic entry through the non dephosphorylation of CDK1 in position 14 and 15 by the CDC25C. Moreover, 14-3-3 binds also to the phosphorylated protein Bad in order to inhibit its pro-apoptotic function because Bad promotes the release of the cytochrome C through its inactivation of Bcl-xL or Bcl-2, leading to apoptosis induction. To conclude, the inhibition of 14-3-3 during RIT treatment could decrease the cell cycle arrest and increase the apoptosis induction driving cells even more sensitive to the radiation damages.

To support this reflexion, in the study of, the therapeutic efficacy of the difopein (a 14-3-3 antagonist) on human glioma cells was studied. The authors showed that this 14-3-3 antagonist had strong effects to induce glioma cell apoptosis through down-regulating Bcl-2, up-regulating Bax and activating caspase-9 and caspase-3. Moreover, the treated cells showed a reduce percentage of cells in G2/M and this inhibitor decreased the tumour xenograft growth compared to control.

### Example 2

Measurement of the effect of combining 177Lu-satetraxetan-lilotomab (Betalutin) with different drugs that regulate the progression through the cell cycle by inhibiting cell cycle proteins

### Materials and Methods

### Cells and Reagents

DLBCL cell lines U2932 and RIVA were maintained in RPMI 1640-GlutaMAX medium (Gibco) supplemented with 15% fetal bovine serum (Biowest) and 1% penicillin-streptomycin (Gibco) at 37° C. in a humidified atmosphere containing 5% CO2. Cells were split twice a week 1:3-1:5 depending on cell density. Betalutin with a specific activity of 600 MBq/mg was prepared by incubation of ¹⁷⁷Lu with p-SCN-benzyl-DOTA conjugated lilotomab for 20 minutes at 37° C. Real Time Glo was purchased from Promega. 384 well plates were purchased from Greiner Bio-One.

### Betalutin Treatment

Cells were treated for 18-20 h with Betalutin at a final concentration of 1 μg/ml and 2 μg/ml in 6-well cell culture plates with shaking. After treatment, PBS was added to the cells, and the cells pelleted. Cells were first resuspended in 1 ml PBS, then washed twice in PBS and finally diluted in growth medium at the desired concentration. All cell lines were treated at a cell concentration of 2.5*10⁶ cells/ml.

Treatment with Inhibitors of Cell Cycle Regulator Proteins and Real Time Glo Viability Assay

Cells density measurements were performed the day before seeding and based on these measurements 2000 cells were seeded in each well in a 384-well plate, which where pre-loaded with selected drugs to result in either 100 nM or 1 μM final concentration. Regardless of cell number the culturing volume was 25 μl. For Real Time Glo, the Cell Viability Substrate and NanoLuc® Enzyme were diluted 1:500 in growth medium, and 25 μL of diluted reagents added to each well. All reagents were equilibrated to 37° C. Cells were incubated with the reaction mix for 1 h at 37° C. before the first measurement of luminescence. Measurements were repeated as often as required within 72 h after adding the reagents. Digitonin was added to cells at 200 μg/ml to record background luminescence of dead cells. A Tecan SPARK 10M plate reader was used to measure luminescence, with an integration time set to 1 sec.

### Data Analysis

Growth inhibition was calculated by dividing the luminescence signal of the treated cells by the luminescence signal of the control cells for each day of measurement after start of treatment [Table 1]. A value of 1 means no change in growth as compared with control cells, a higher value represents increased growth and a lower value means inhibition of growth.

To evaluate effects of the drugs alone [(−)Betalutin] and their combination with Betalutin [(+)Betalutin], we calculated the Z-score for each sample (ZdrugX=1-(+)BetalutindrugX/(−)BetalutindrugX), where (+)BetalutindrugX and (−) BetalutindrugX are the measured luminescence intensity values for drug X in presence and absence of Betalutin, respectively. The calculation of Z-score values separately for each plate enabled comparison of results across plates, despite variations between the plates in overall signal intensity. Drugs with a Z-score that is 2×STDEV higher than the mean effect of Betalutin alone (Zcontrol=1−(+)Betalutincontrol/(−)Betalutincontrol) at one of the four consecutive days was considered as a hit [Z-score Table 2].

To identify greater than additive effects between Betalutin and the drugs, we calculated the expected additive effect to compare it to the measured effect (Bliss independence test; expected additive effect: FBetalutin&Drug=FBetalutin+FDrug−FBetalutin×FDrug). Drugs with a score (“measured effect−expected additive effect”) which is 2×STDEV higher than the mean effect of Betalutin alone at one of the four consecutive days was considered a hit [Bliss-score Table 2].

### Results

TABLE 1
Growth inhibition of U2932 and RIVA cells after treatment with Betalutin and cell cycle
inhibitors alone and in combination. Dy = Day after start of treatment.

| | | Dy3 | Dy4 | Dy5 | Dy6 | Dy3 | Dy4 | Dy5 | Dy6 |
|---|---|---|---|---|---|---|---|---|---|
| U2932 | Drug 100 nM | (−)BETALUTIN | | | | (+)BETALUTIN | | | |
| | MK-1775 | 1.00 | 0.90 | 0.92 | 1.03 | 1.20 | 0.95 | 0.85 | 0.71 |
| | AMG 900 | 0.96 | 0.97 | 0.92 | 0.72 | 0.89 | 0.78 | 0.77 | 0.52 |
| | AZD7762 | 1.01 | 1.03 | 0.97 | 1.03 | 1.00 | 0.87 | 0.76 | 0.62 |
| | Mean controls | 1.00 | 1.00 | 1.00 | 1.00 | 1.08 | 0.90 | 0.87 | 0.72 |
| | CYC116 | 0.94 | 0.97 | 1.06 | 1.21 | 0.92 | 0.94 | 0.80 | 0.75 |
| | JNJ-7706621 | 0.90 | 0.91 | 1.07 | 1.16 | 0.83 | 0.82 | 0.73 | 0.72 |
| | Mean controls | 1.00 | 1.00 | 1.00 | 1.00 | 0.95 | 0.99 | 0.86 | 0.81 |
| U2932 | Drug 1 μM | (−)BETALUTIN | | | | (+)BETALUTIN | | | |
| | MK-1775 | 0.86 | 0.89 | 0.96 | 1.15 | 1.04 | 0.94 | 0.89 | 0.80 |
| | AMG 900 | 0.83 | 0.96 | 0.96 | 0.81 | 0.77 | 0.77 | 0.80 | 0.58 |
| | AZD7762 | 0.04 | 0.03 | 0.03 | 0.03 | 0.03 | 0.02 | 0.02 | 0.02 |
| | Mean controls | 1.00 | 1.00 | 1.00 | 1.00 | 0.93 | 0.92 | 0.94 | 0.81 |
| | CYC116 | 0.85 | 0.89 | 0.83 | 0.61 | 0.72 | 0.82 | 0.74 | 0.56 |
| | JNJ-7706621 | 0.53 | 0.36 | 0.25 | 0.20 | 0.39 | 0.26 | 0.16 | 0.11 |
| | Mean controls | 1.00 | 1.00 | 1.00 | 1.00 | 0.96 | 0.98 | 0.88 | 0.75 |
| RIVA | Drug 100 nM | −BETALUTIN | | | | +BETALUTIN | | | |
| | MK-1775 | 1.00 | 0.89 | 0.94 | 0.94 | 0.49 | 0.38 | 0.24 | 0.23 |
| | AMG 900 | 0.82 | 0.85 | 0.65 | 0.51 | 0.22 | 0.16 | 0.09 | 0.08 |
| | AZD7762 | 0.59 | 0.60 | 0.68 | 0.68 | 0.52 | 0.48 | 0.34 | 0.34 |
| | Mean controls | 1.00 | 1.00 | 1.00 | 1.00 | 0.17 | 0.12 | 0.07 | 0.06 |
| | CYC116 | 0.93 | 0.94 | 1.14 | 0.92 | 0.13 | 0.10 | 0.06 | 0.06 |
| | JNJ-7706621 | 0.89 | 0.82 | 0.99 | 0.89 | 0.13 | 0.08 | 0.04 | 0.04 |
| | Mean controls | 1.00 | 1.00 | 1.00 | 1.00 | 0.15 | 0.11 | 0.07 | 0.06 |
| RIVA | Drug 1 μM | −BETALUTIN | | | | +BETALUTIN | | | |
| | MK-1775 | 0.08 | 0.04 | 0.02 | 0.01 | 0.02 | 0.01 | 0.01 | 0.01 |
| | AMG 900 | 0.60 | 0.57 | 0.49 | 0.40 | 0.09 | 0.08 | 0.05 | 0.04 |
| | AZD7762 | 0.03 | 0.02 | 0.02 | 0.01 | 0.01 | 0.01 | 0.01 | 0.01 |
| | Mean controls | 1.00 | 1.00 | 1.00 | 1.00 | 0.16 | 0.11 | 0.08 | 0.07 |
| | CYC116 | 0.62 | 0.78 | 0.70 | 0.65 | 0.22 | 0.20 | 0.14 | 0.14 |
| | JNJ-7706621 | 0.50 | 0.31 | 0.17 | 0.14 | 0.29 | 0.13 | 0.04 | 0.03 |
| | Mean controls | 1.00 | 1.00 | 1.00 | 1.00 | 0.18 | 0.13 | 0.09 | 0.08 |

TABLE 2
Overview of synergy for the combination between
Betalutin and cell cycle inhibitors. Bliss = Synergy is
indicated with the Bliss method. Z-score = Synergy is
indicated with the Z-score method. Bliss + Z-score =
Synergy is indicated with both methods.

| | U2932 | | RIVA | |
|---|---|---|---|---|
| Drug | 100 nM | 1 μM | 100 nM | 1 μM |
| MK-1775 | — | — | — | — |
| AMG 900 | Bliss + Z-score | Z-score | — | — |
| AZD7762 | Bliss + Z-score | — | — | — |
| CYC116 | Bliss + Z-score | Z-score | — | — |
| JNJ-7706621 | Bliss | — | Bliss + Z-score | Z-score |

### Conclusion

Inhibition of cell cycle regulatory enzymes, such as WEE1, CHK1, CDK and AURORA-kinases, by specific inhibitors significantly potentiates the cell proliferation inhibitory effect of Betalutin in two aggressive Diffuse Large B Cell Lymphoma cell lines.

Hence, inhibition of cell cycle regulatory enzymes in combination with Betalutin appear as a novel and promising avenue to investigate for the treatment of difficult to treat B cell lymphoma.

### Example 3—Combination of Humalutin and Olaparib can be Synergistic

### Objective

The combination treatment with External Beam Radiation and the PARP inhibitor olaparib can be synergistic. In the present example the aim is to explore if the combination of the radioimmunoconjugate Humalutin, (chHH1.1-satetraxetan labelled with ¹⁷⁷Lu) as a vehicle to deliver selectively radiation to tumor cells, and the PARP inhibitor olaparib is also synergistic.

### Materials and Methods

### Cell Lines

The cells were grown in RPMI 1640 medium and DMEM culture media supplemented with Glutamax (Gibco, Paisley, UK), 10% heat activated FBS (Gibco) and 1% penicillin-streptomycin (Gibco). The cells were cultured at 37° C. and 5% CO₂. Cell suspensions were diluted 1:3, 1:4 or 1:5 with pre-heated medium twice a week and diluted 2-4 days before start of the experiment, to ensure they are in exponential growth at the beginning of the experiment.

TABLE 3
Cell lines used, particulars and culture conditions.

| | Lymphoma | Relevant | |
|---|---|---|---|
| Cell line | type | information | medium |
| REC-1 | MCL | Functional | RPMI 1640, 100 U/ml |
| | | ATM | penicillin/streptomycin, |
| | | | 10% FBS |
| Granta 519 | MCL | Partial | DMEM (high glucose), |
| | | ATM loss | 100 U/ml |
| | | | penicillin/streptomycin, |
| | | | 10% FBS |
| DOHH2 | FL transformed to | t(14; 18), BH3 | RPMI 1640, 100 U/ml |
| | GCB-DLBCL | sensitive | penicillin/streptomycin, |
| | | | 10% FBS |
| SUDHL4 | FL transformed to | t(14; 18), BH3 | RPMI 1640, 100 U/ml |
| | GCB-DLBCL | insensitive | penicillin/streptomycin, |
| | | | 10% FBS |
| U2932 | ABC-DLBCL | BCL2, BCL6 | RPMI 1640, 100 U/ml |
| | | overexpression, | penicillin/streptomycin, |
| | | BH3 sensitive | 10% FBS |
| WSU- | GCB-DLBCL | t(14; 18), BH3 | RPMI 1640, 100 U/ml |
| DLCL2 | | insensitive | penicillin/streptomycin, |
| | | | 10% FBS |

MCL: Mantle Cell Lymphoma
GCB: Germinal Center B-cell
ABC: Activated B-cell
DLBCL: Diffuse Large B-Cell Lymphoma
FL: Folicular Lymphoma

Labeling of chHH1.1-Satetraxetan with ¹⁷⁷Lu

The chelator p-SCN-Bn-DOTA (satetraxetan, Macrocyclics, TX, USA) was dissolved in 0.005 M HCl, added to the antibody in a 6:1 ratio and pH-adjusted to approximately 8.5 using carbonate buffer. After 45 minutes of incubation at 37° C. the reaction was stopped by the addition of 50 μl per mg of Ab of 0.2 M glycine solution. To remove free satetraxetan the conjugated antibody was washed using Vivaspin 20 centrifuge tubes (Sartorius Stedim Biotech, Gottingen Germany) 4-5 times with NaCl 0.9%. Before labeling with ¹⁷⁷Lu the pH was adjusted to 5.3±0.3 using 0.25 M ammonium acetate buffer. Around 200 MBq of ¹⁷⁷Lu (ITG, Garching, Germany) was added to 0.25 mg of satetraxetan-chHH1, and incubated for 15 to 30 minutes at 37° C. The radiochemical purity (RCP) of the conjugate was evaluated using instant thin layer chromatography and was higher than 95%. The specific activity was set at 600 MBq/mg (dilution with cold chHH1-satetraxetan was done as required).

### Immunoreactive Fraction of Humalutin

The immunoreactivity of the radioimmunoconjugates was measured using Ramos cells and a one point modified Lindmo method. The cell concentration used was 75 million cells/ml. The immunoreactivity of the conjugates was higher than 70%.

### Cytotoxicity Studies

Cells were treated with either Humalutin, olaparib or a combination of both and seeded into 96 well-plates. At each time point after treatment, cells were incubated with Alamar Blue (Thermo Fisher, DALL1100) for 4 hours and fluorescence measurements were performed using a multiplate reader Fluoroskan ascent FL to assess cell proliferation and viability. All experiments were done in duplicates using 2 samples in each experiment. Data were normalized to the untreated controls. The IC50 was calculated using a log scale transform and non-linear fit with top at 100% and bottom at 0%.

Treatment with Humalutin

Cells were incubated with 0.25, 0.5, 1, 2.5 or 5 μg/ml of Humalutin in cell culture flasks and incubated at 37° C./5% CO₂. After 18-20 hours cells were washed and resuspended in fresh medium. Cell bound activity after washing was measured using a calibrated gamma detector (Cobra II auto-gamma detector, Packard Instrument Company, Meriden, Conn., USA). Cell concentration and viability was also measured after washing using Guava ViaCount Cell Dispersal Reagent for Flow Cytometry (Merck GaA, Darmstadt, Germany) and measured in a Guava EasyCyte 12HT (Merck KGaA, Darmstadt, Germany) to determine how well the cells survived the incubation. Cells were seeded in 96 well plates.

Treatment with Olaparib

Cells were seeded in 96 well plates pre-coated with from 1 to 100 μM olaparib in 0.2 ml medium and incubated at 37° C./5% CO₂ for 72 hours before the cytotoxic effect was measured using alamar blue cell viability assay.

Treatment with Humalutin and Olaparib

Cells were incubated with either 0, 0.5 μg/ml or 1 μg/ml Humalutin and the same procedure as described before for treatment with humalutin alone and olaparib alone was followed. Concentrations of olaparib used were between 1 and 100 μM.

### Statistics

The Chou-Talalay model was used for synergy calculations using the Compusyn software. R (goodness of fit) was calculated for the individual treatments and should be over 0.90 in in vitro culture experiments in order to use the calculated combination index (CI). The CI is an indication of synergy: 0-0.9 is considered synergy. Synergysm grading was used as described in WO2006004917A2.

### Results and Discussions

### Humalutin and Olaparib Alone

The sensitivity to Humalutin and olaparib varied among the different cell lines (Table 4). Among the most sensitive to olaparib were Rec-1 and SDUHL4 while DOHH2 and Granta 519 were among the most resistant. The most sensitive cell lines to Humalutin were Granta 519 and SUDHL4, while the most radioresistant were WSU-DLCL2 and Rec-1.

TABLE 4
IC50 values for Humalutin
and olaparib after 72 h
incubation time after seeding.

| | IC50 | | IC50 | |
|---|---|---|---|---|
| | Humalutin | R value | olaparib | R value |
| Cell line | (μg/ml) | Humalutin | (μM) | olaparib |
| DOHH2 | 9.205 | 0.9433 | 2.06 | 0.9924 |
| Granta 519 | 2.545 | 0.99 | 2.681 | 0.792 |
| Rec-1 | 21.03 | 0.9761 | 0.7206 | 0.9279 |
| SUDHL4 | 6.484 | 0.4762 | 1.045 | 0.9173 |
| U2932 | 12.42 | 0.345 | 1.929 | 0.1376 |
| WSU-DLCL2 | 19.06 | 0.8859 | 1.261 | 0.6319 |

### Combination of Humalutin and Olaparib

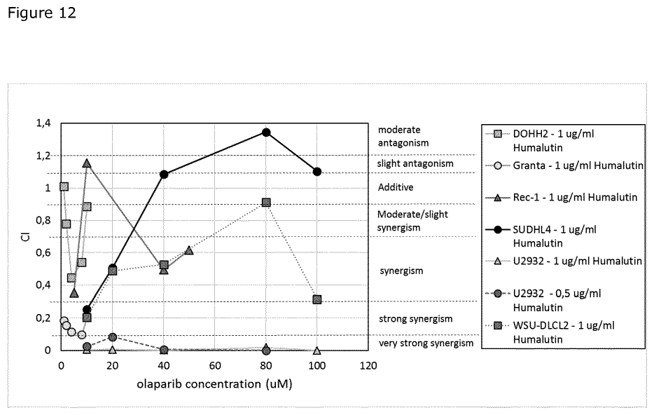

Calculation of the Combination Index (CI) by the Chou Talalay method showed synergism for most cell lines (FIG. 12). U2932 and Granta 519 showed very strong synergism for all olaparib concentrations. The rest of the cell lines showed varying degrees of synergism depending on the dose of olaparib used. In addition, SUDHL4 showed moderate antagonism for one of the olaparib concentrations tested. It is important to notice that the R values for the treatments alone were below 0.9 in some of the cell lines, which means that the results from the Chou Talalay method should be considered with care. Further repetitions of the experiment will be performed in the future to reach higher R values for the independent treatments.

Granta 519, a MCL with partial loss of ATM functionality showed low sensitivity to olaparib alone, while the synergy between Humalutin and olaparib was strong. On the other hand, the DLBCL cell line U2932 was not very sensitive to Humalutin but the synergy with olaparib was very strong. These results warrant further studies in animal models to investigate if the same synergy can be seen in vivo.

### Conclusions

Synergy between Humalutin and the PARP inhibitor olaparib was observed in all cell lines tested, with a tendency to stronger synergism at lower olaparib concentrations. Results indicate that treatment with radioimmunotherapy can sensitize lymphoma to PARP inhibitors. Further studies in animal models are warranted.

### Example 4—Combination of Betalutin and PARP Inhibitors AG-14361 and Rucaparib can Reverse Betalutin Resistance in Aggressive ABC-Like Diffuse Large B Cell Lymphoma Cell Lines U-2932 and RIVA

### Objective

Diffuse Large B-cell Lymphoma (DLBCL) is an aggressive form of Non-Hodgkin Lymphoma (NHL). The applicant is currently developing a potential targeted therapy for recurrent NHL with the antibody-radionuclide conjugate (ARC) Betalutin. U-2932 and RIVA are two Activated B-cell like DLBCL cell lines that show resistance to Betalutin treatment at clinically relevant doses. The inventors have in previously examples found that the combination treatment with External Beam Radiation and the PARP inhibitor olaparib can be synergistic. In the current study the inventors aim to explore if the combination of the radioimmunoconjugate Betalutin, as a vehicle to deliver selectively radiation to tumor cells, and selected other PARP inhibitors can reverse resistance to Betalutin treatment. To this, cells will be pre-treated with Betalutin (or not), before removal of excess Betalutin, and seeding onto 384-well plates pre-loaded with selected drugs from the Selleck Cancer Compound library. We aim to determine if drugs synergize with Betalutin to reduce viability as measured by Real Time Glo.

### Materials and Methods

### Cell Lines

DLBCL cell lines U2932 and RIVA were maintained in RPMI 1640-GlutaMAX medium (Gibco) supplemented with 15% fetal bovine serum (Biowest) and 1% penicillin-streptomycin (Gibco) at 37° C. in a humidified atmosphere containing 5% CO2. Cells were split twice a week 1:7 and diluted 2-4 days before start of the experiment, to ensure they are in exponential growth at the beginning of the experiment.

TABLE 5
Cell lines used

| | Lymphoma | | |
|---|---|---|---|
| Cell line | type | Relevant information | medium |
| U2932 | ABC-DLBCL | BCL2, BCL6, TP53, | RPMI 1640, |
| | | RB1 over-expression, | 100 U/ml |
| | | TP53 mutation | penicillin/ |
| | | | streptomycin, |
| | | | 15% FBS |
| RIVA | ABC-DLBCL | t(4; 8) MYC rearrangement, | RPMI 1640, |
| | | der(18) BCL2 amplifi-cation, | 100 U/ml |
| | | P15INK4B deletion, | penicillin/ |
| | | P16INK4A deletion, RB1 | streptomycin, |
| | | deletion/mutation | 15% FBS |

ABC-DLBCL: Activated B-cell like Diffuse Large B-Cell Lymphoma

Labeling of chHH1-Satetraxetan with ¹⁷⁷Lu

The chelator p-SCN-Bn-DOTA (satetraxetan, Macrocyclics, TX, USA) was dissolved in 0.005 M HCl, added to the antibody in a 6:1 ratio and pH-adjusted to approximately 8.5 using carbonate buffer. After 45 minutes of incubation at 37° C. the reaction was stopped by the addition of 50 μl per mg of Ab of 0.2 M glycine solution. To remove free satetraxetan the conjugated antibody was washed using Vivaspin 20 centrifuge tubes (Sartorius Stedim Biotech, Gottingen Germany) 4-5 times with NaCl 0.9%. Before labeling with ¹⁷⁷Lu the pH was adjusted to 5.3±0.3 using 0.25 M ammonium acetate buffer. Around 200 MBq of ¹⁷⁷Lu (ITG, Garching, Germany) was added to 0.25 mg of satetraxetan-chHH1, and incubated for 15 to 30 minutes at 37° C. The radiochemical purity (RCP) of the conjugate was evaluated using instant thin layer chromatography and was higher than 95%. The specific activity was set at 600 MBq/mg (dilution with cold chHH1-satetraxetan was done as required).

### Immunoreactive Fraction of Betalutin

The immunoreactivity of the radioimmunoconjugates was measured using Ramos cells and a one point modified Lindmo method. The cell concentration used was 75 million cells/ml. The immunoreactivity of the conjugates was higher than 70%.

### Cytotoxicity Studies

### Betalutin Treatment

Cells were treated in 6-well plates without shaking for 18 h with Betalutin at a final concentration of 1 μg/ml for U2932 and 0.5 μg/ml for RIVA. After treatment, PBS was added to the cells, and the cells pelleted. Cells were first resuspended in 1 ml PBS, then washed twice in PBS and finally diluted in growth medium to a final concentration of 2.5*10⁶ cells/ml.

### Real Time Glo Viability Assays.

Cells density measurements were performed the day before seeding. Based on these measurements cells were seeded in 384-well plates at a density of 3000 cells per well in a culturing volume of 25 μl (resulting in start titers: 120 000 cells/ml) using a robot. For measuring viability using Real Time Glo (Promega, WI, USA), the Cell Viability Substrate and NanoLuc® Enzyme were diluted 1:500 in growth medium, and 25 μL of diluted reagents dispensed in each well by a robot. All reagents were equilibrated to 37° C. Cells were incubated with the reaction mix for 1 h at 37° C. before the first measurement of luminescence. Measurements were repeated as often as required within 72 h after adding the reagents. A Tecan SPARK 10M plate reader (Tecan, SUI) was used to measure luminescence, with the integration time set to 1 sec.

### Plate Setup

The screen was performed in 384-well plate with selected PARP inhibitors (stock solutions 10 mM in DMSO) acquired from SelleckChem (Selleckchem, TX, USA). To include no-drug controls, the drug panel (Table 6) was divided on two plates. Due to previous observations of cells growing poorly in wells located at the edges of the plates, the two outer most rows and columns were not used. As previously mentioned three different drug concentrations were used in the screen, 10 nM and 1 μM for U2932 and 10 and 100 nM for RIVA.

TABLE 6

| Inhibitor | |
|---|---|
| Name | alternative names |
| Olaparib | AZD2281, Ku-0059436 |
| Veliparib | ABT-888 |
| Rucaparib | AG-0146999, PF-01367338 |
| AG-14361 | |
| Iniparib | BSI-201 |
| AZD2461 | |

### Data Analysis

Candidate hits were identified using the Bliss Independence test for synergy. The effect of each drug alone at each concentration was calculated as the fraction of dead cells as compared to control cells Fₐ=(1−(RLU_{drug}/avgRLU_{control}), a similar calculation was performed for the effect of Betalutin alone F_{b}=(1−(avgRLU_{Betalutin}/avgRLU_{control}). Through the following equation we found the expected additive effect of the combination of drug+Betalutin: Expected effect (E)F_{ab}=(Fₐ+F_{b}−Fₐ*F_{b}). We subtracted this expected effect from the measured effect (M)F_{ab}=(1−(RLU_{Drug+Betalutin}/avgRLU_{control}) to get a value (Diff)F_{ab} representing how the measured effect differed from the expected additive effect of the combination. This value was normalized to the survival fraction of drug alone: (Diff)F_{ab}#=(Diff)F_{ab}/(1−Fₐ). Furthermore, to get a measure of how big the well-to-well variation was, we calculated the standard deviation for the effect of Betalutin alone in the 48 control wells on each plate: (STDEV)F_{b}=(RLU_{Betalutin}/RLU_{control})−(averageRLU_{Betalutin}/averageRLU_{control}). Drugs with a value (Diff)F_{ab}#two times higher than the standard deviation of Betalutin treated controls, (STDEV)F_{b}, were scored as hits.

### Results and Discussions

### Betalutin Alone

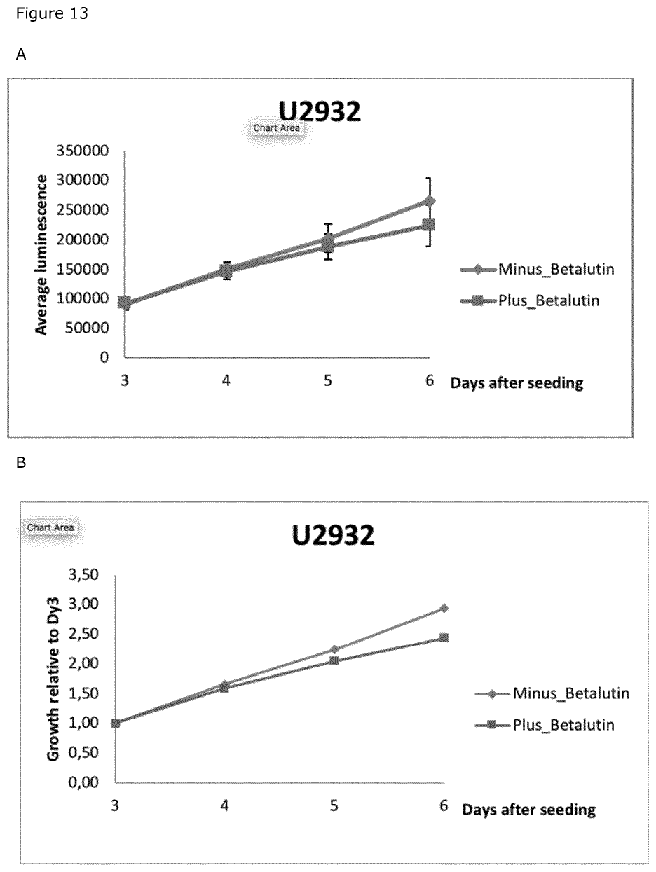

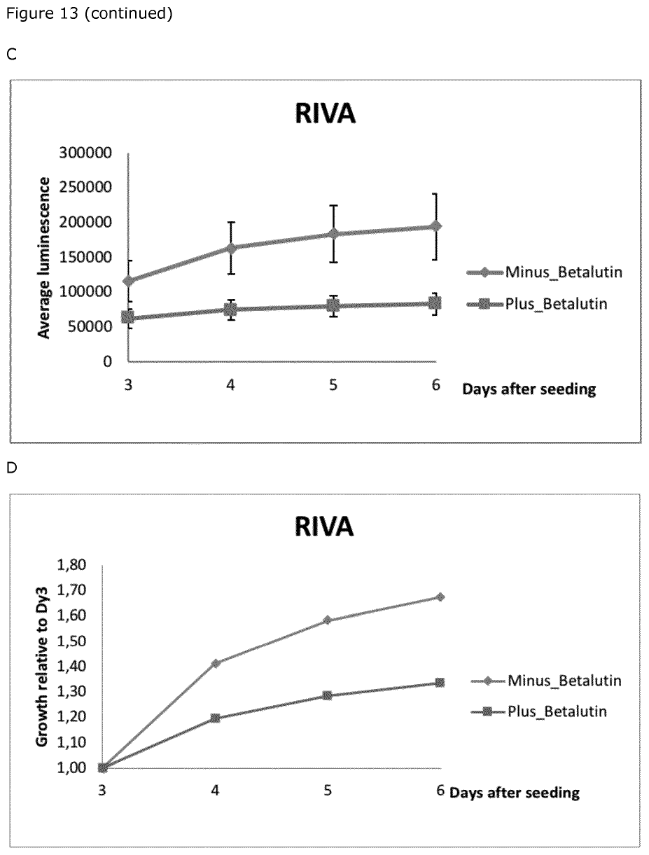

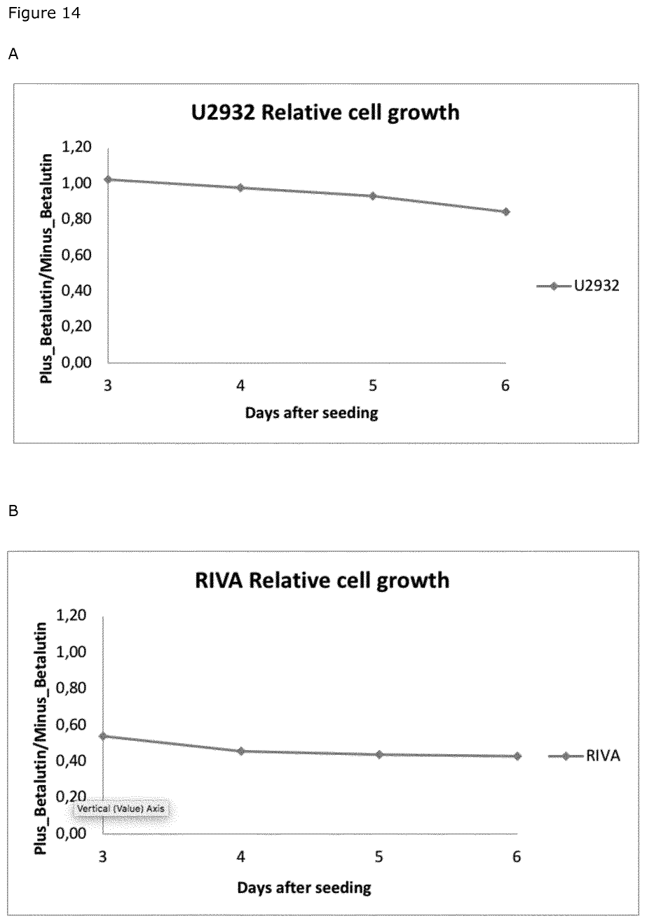

The sensitivity to Betalutin differed between U-2932 and RIVA cells. U-2932 cells were more resistant to Betalutin than RIVA cells (FIG. 1). U-2932 cells proliferated upon treatment with 1 μg/ml [600 MBq/mg] at more than 80% of the level of non-treated U2932 cells (FIG. 1A). RIVA cell had higher sensitivity to Betalutin than U-2932, but also proliferated at more than 40% of the rate of non-treated cells throughout the observation window of 4-6 days (treatment 0.5 μg/ml[600 MBq/mg]) (FIG. 13B). FIG. 14 shows the relative proliferation (luminescence) of Betalutin treated cells normalized to the proliferation rate of untreated cells at the respective days.

AG-14361 or Rucaparib Alone and Combination with Betalutin

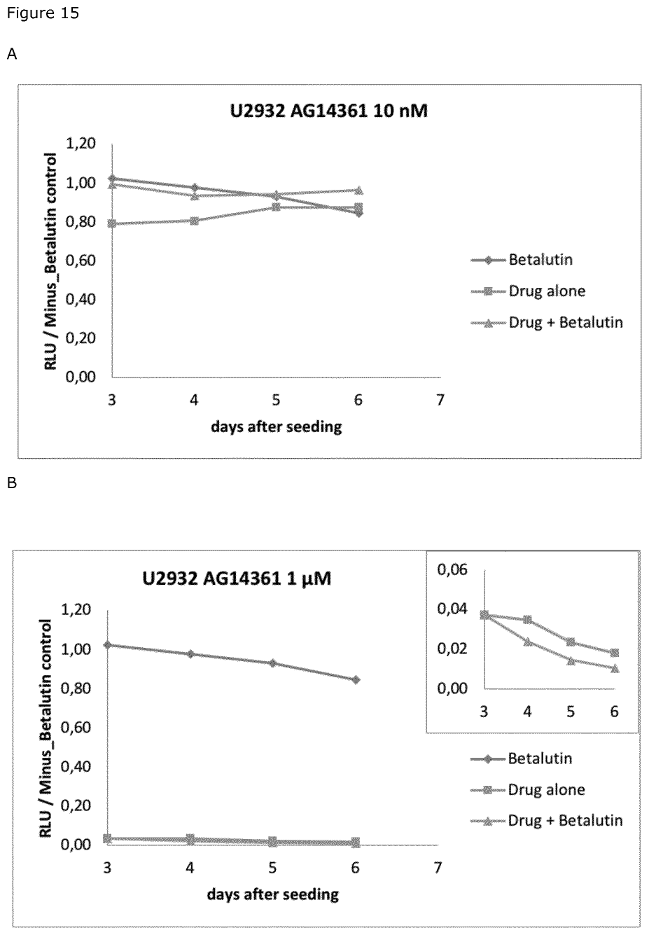

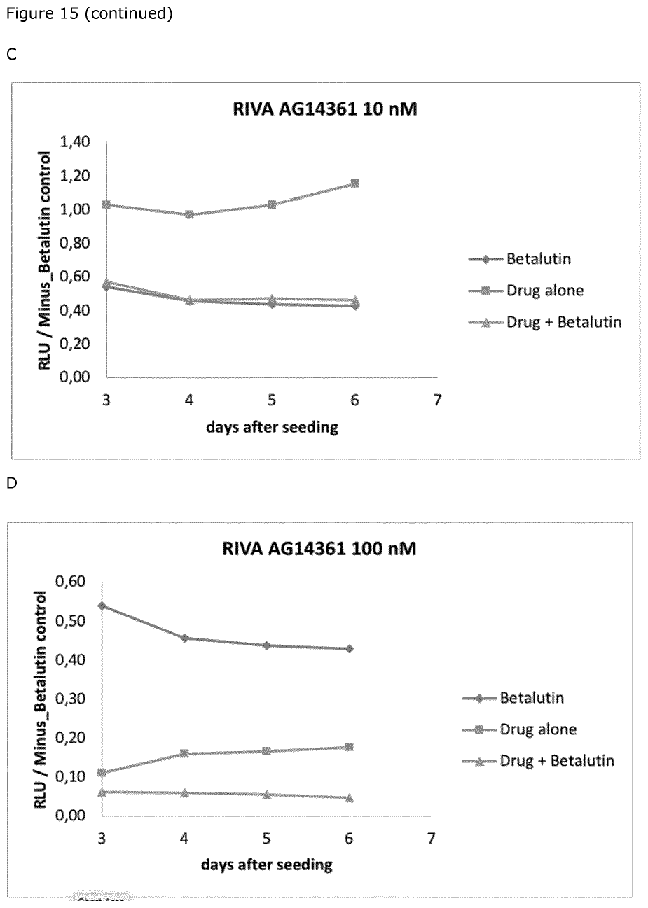

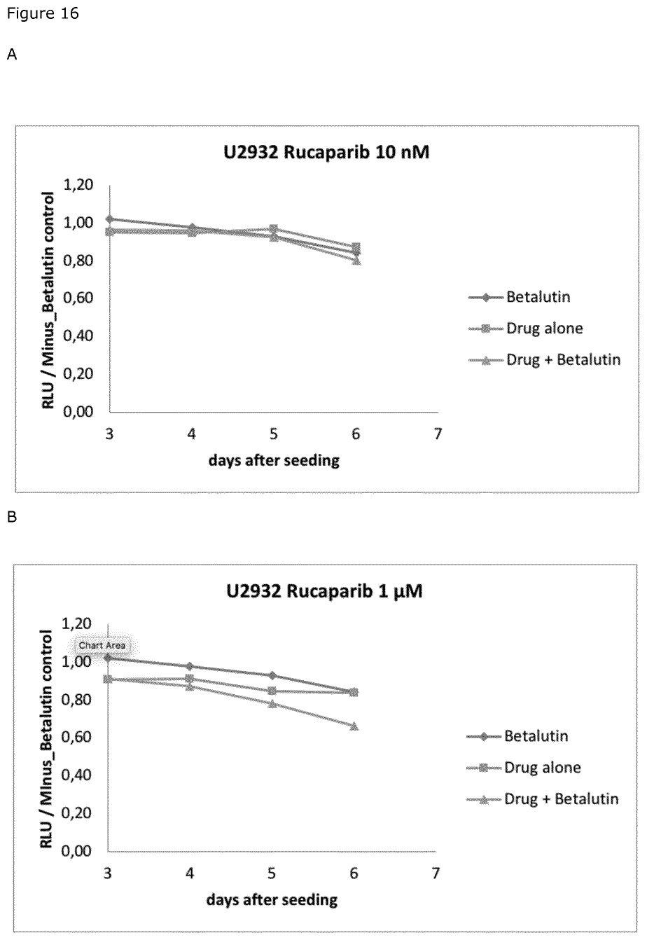

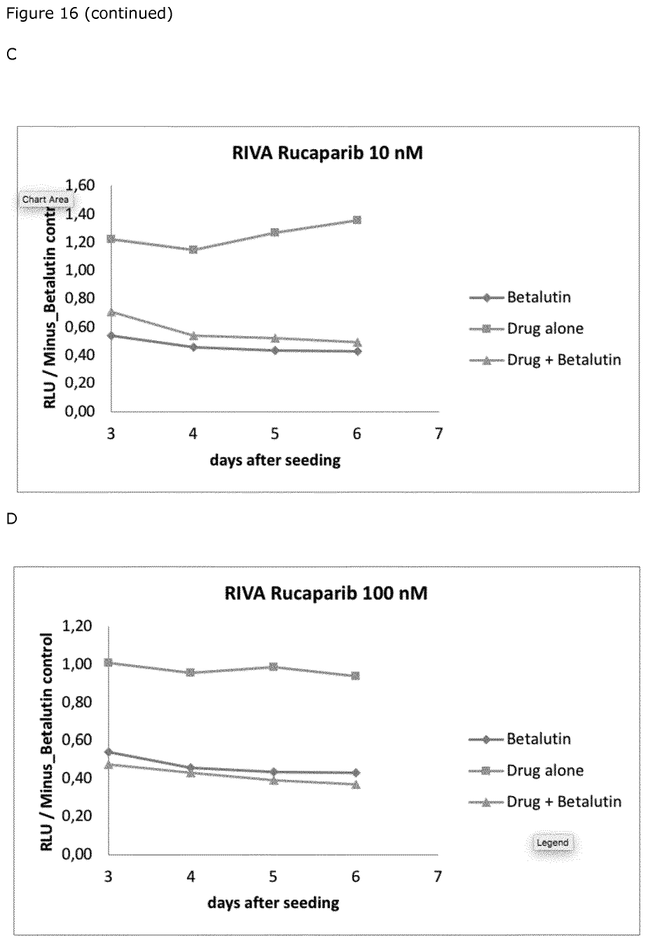

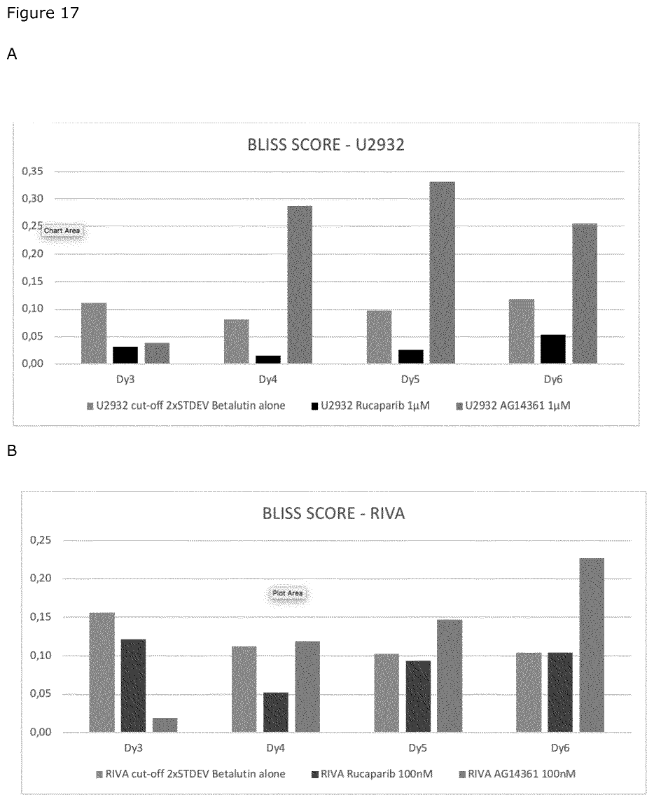

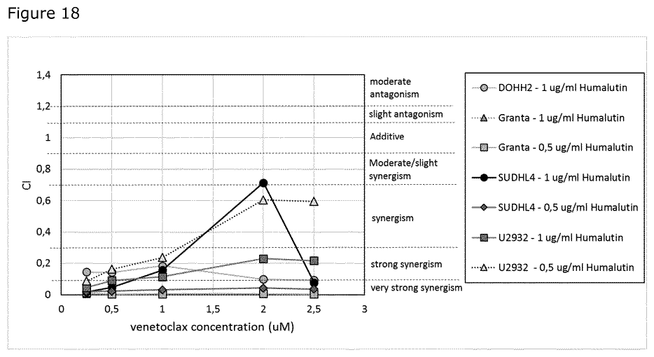

Treatment of U-2932 or RIVA cells with AG-14361 at 10 nM had no growth inhibitory effect when given alone and did not enhance the growth inhibitory effect of Betalutin (FIG. 15A,C). In contrast, AG-14361 inhibited proliferation of U-2932 and RIVA cells at 1 μM and 100 nM, respectively. Combination of AG-14361 with Betalutin showed stronger growth inhibition than AG-14361 alone (FIG. 15B,D). Treatment of U-2932 or RIVA cells with Rucaparib alone had no growth inhibitory effect at the tested concentrations (FIG. 16). Combination of Rucaparib with Betalutin showed stronger growth inhibition than Betalutin alone (FIG. 16 B,D).

The resulting growth inhibition of the tested PARP inhibitors (RIVA 100 nM; U2932 1 μM) in combination with Betalutin is larger than their expected additive effect. AG-14361 shows the strongest combinatory effect and scores at days 4, 5, and 6 with an effect size larger than two standard-deviations of Betalutin treated cells alone (BLISS test, FIG. 17).

### Conclusions

Combination of Betalutin with the PARP inhibitor AG-14361 overcomes Betalutin resistance in two cell lines of ABC-like Diffuse Large B cell lymphoma and results in growth inhibition with a more than expected additive effect, indicative for synergism. Combination of Betalutin and Rucaparib also increased the growth inhibitory effect to an extent larger than the expected additive effect, but at lower efficacy than AG-14361 under the tested concentrations.

These results indicate that combination treatment of PARP inhibitors with Betalutin radioimmunotherapy can increase the sensitivity of Betalutin resistant aggressive diffuse large B cell lymphoma. Further studies in animal models are warranted.

### Example 5 Combination of Humalutin and Venetoclax can be Synergistic

### Objective

The combination treatment with External Beam Radiation and the BH3 mimetic venetoclax can be synergistic. In the present example the aim is to explore if the combination of the radioimmunoconjugate Humalutin, (chHH1-satetraxetan labelled with ¹⁷⁷Lu) as a vehicle to deliver selectively radiation to tumor cells, and the BH3 mimetic venetoclax is also synergistic.

### Materials and Methods

### Cell Lines

The cells were grown in RPMI 1640 medium and DMEM culture media supplemented with Glutamax (Gibco, Paisley, UK), 10% heat activated FBS (Gibco) and 1% penicillin-streptomycin (Gibco). The cells were cultured at 37° C. and 5% CO₂.

Cell suspensions were diluted 1:3, 1:4 or 1:5 with pre-heated medium twice a week and diluted 2-4 days before start of the experiment, to ensure they were in exponential growth at the beginning of the experiment.

TABLE 7
Cell lines used, particulars and culture conditions.

| | Lymphoma | Relevant | |
|---|---|---|---|
| Cell line | type | information | medium |
| Granta- | MCL | Partial | DMEM |
| 519 | | ATM loss | (high glucose), |
| | | | 100 U/ml |
| | | | penicillin/ |
| | | | streptomycin, |
| | | | 10% FBS |
| DOHH2 | FL | t(14; 18), | RPMI 1640, |
| | transformed to | BH3 | 100 U/ml |
| | GCB-DLBCL | sensitive | penicillin/ |
| | | | streptomycin, |
| | | | 10% FBS |
| SUDHL4 | FL | t(14; 18), | RPMI 1640, |
| | transformed to | BH3 | 100 U/ml |
| | GCB-DLBCL | insensitive | penicillin/ |
| | | | streptomycin, |
| | | | 10% FBS |
| U2932 | ABC-DLBCL | BCL2, BCL6 | RPMI 1640, |
| | | overexpression, | 100 U/ml |
| | | BH3 sensitive | penicillin/ |
| | | | streptomycin, |
| | | | 10% FBS |

MCL: Mantle Cell Lymphoma
GCB: Germinal Center B-cell
ABC: Activated B-cell
DLBCL: Diffuse Large B-Cell Lymphoma
FL: Folicular Lymphoma

Labeling of chHH1-Satetraxetan with ¹⁷⁷Lu

The chelator p-SCN-Bn-DOTA (satetraxetan, Macrocyclics, TX, USA) was dissolved in 0.005 M HCl, added to the antibody in a 6:1 ratio and pH-adjusted to approximately 8.5 using carbonate buffer. After 45 minutes of incubation at 37° C. the reaction was stopped by the addition of 50 μl per mg of Ab of 0.2 M glycine solution. To remove free satetraxetan the conjugated antibody was washed using Vivaspin 20 centrifuge tubes (Sartorius Stedim Biotech, Gottingen Germany) 4-5 times with NaCl 0.9%. Before labeling with ¹⁷⁷Lu the pH was adjusted to 5.3±0.3 using 0.25 M ammonium acetate buffer. Around 200 MBq of ¹⁷⁷Lu (ITG, Garching, Germany) was added to 0.25 mg of satetraxetan-chHH1, and incubated for 15 to 30 minutes at 37° C. The radiochemical purity (RCP) of the conjugate was evaluated using instant thin layer chromatography and was higher than 95%. The specific activity was set at 600 MBq/mg (dilution with cold chHH1-satetraxetan was done as required).

### Immunoreactive Fraction of Humalutin

The immunoreactivity of the radioimmunoconjugates was measured using Ramos cells and a one point modified Lindmo method (1, 2). The cell concentration used was 75 million cells/ml. The immunoreactivity of the conjugates was higher than 70%.

### Cytotoxicity Studies

Cells were treated with either Humalutin, venetoclax or a combination of both and seeded into 96 well-plates. At each time point after treatment, cells were incubated with Alamar Blue (Thermo Fisher, DALL1100) for 4 hours and fluorescence measurements were performed using a multiplate reader Fluoroskan ascent FL to assess cell proliferation and viability. All experiments were done in duplicates using 2 samples in each experiment. Data were normalized to the untreated controls. The IC50 was calculated using a log scale transform and non-linear fit with top at 100% and bottom at 0%.

Treatment with Humalutin

Cells were incubated with 0.25, 0.5, 1, 2.5 or 5 μg/ml of Humalutin in cell culture flasks and incubated at 37° C./5% CO₂. After 18-20 hours cells were washed and resuspended in fresh medium. Cell bound activity after washing was measured using a calibrated gamma detector (Cobra II auto-gamma detector, Packard Instrument Company, Meriden, Conn., USA). Cell concentration and viability was also measured after washing using Guava ViaCount Cell Dispersal Reagent for Flow Cytometry (Merck GaA, Darmstadt, Germany) and measured in a Guava EasyCyte 12HT (Merck KGaA, Darmstadt, Germany) to determine how well the cells survived the incubation. Cells were seeded in 96 well plates.

Treatment with Venetoclax

Cells were seeded in 96 well plates pre-coated with 0, 0.5, 1, 2 and 2.5 μM venetoclax in 0.2 ml medium and incubated at 37° C./5% CO₂ for 72 before the cytotoxic effect was measured using alamar blue cell viability assay.

Treatment with Humalutin and Venetoclax

Cells were incubated with either 0, 0.5 μg/ml or 1 μg/ml Humalutin and the same procedure as described before for treatment with Humalutin alone and venetoclax alone was followed. Concentrations of venetoclax were set at 0, 0.5, 1, 2 and 2.5 μM.

### Statistics

The Chou-Talalay model was used for synergy calculations using the Compusyn software. R (goodness of fit) was calculated for the individual treatments and should be over 0.90 in in vitro culture experiments in order to use with confidence the calculated combination index (CI). The CI is an indication of synergy: 0-0.9 is considered synergy. Synergysm grading was used as described in WO2006004917A2.

### Results and Discussions

### Humalutin and Venetoclax Alone

The sensitivity to humalutin and venetoclax varied among the different cell lines (Table 8). Granta 519 was the most sensitive cell line to both treatments, while U2932 was the most resistant cell line to both treatments.

TABLE 8
IC50 values for Humalutin and venetoclax after
72 h incubation time after seeding.

| | IC50 | | IC50 | |
|---|---|---|---|---|
| | Humalutin | R value | venetoclax | R value |
| Cell line | (μg/ml) | Humalutin | (μM) | venetoclax |
| DOHH2 | 9.205 | 0.9433 | 2.975 | 0.9012 |
| Granta 519 | 2.545 | 0.99 | 0.3306 | 0.7583 |
| SUDHL4 | 6.484 | 0.4762 | 3.662 | 0.8608 |
| U2932 | 12.42 | 0.345 | 4.147 | 0.9602 |

### Combination of Humalutin and Venetoclax

Calculation of the Combination Index (CI) by the Chou Talalay method showed synergism for all cell lines.

Most cell lines showed very strong to strong synergism, with a tendency to stronger synergism at lower venetoclax concentrations. The highest synergy was observed for Granta 519 and SUDHL4 cell lines. It is of interest to notice that U2932 was the most resistant cell line to both treatments but showed strong synergy for the combination. These results indicate that treatment with radiation can sensitize DLBCL and MCL cells to BH3 mimetics.

### Conclusions

Synergy between Humalutin and the BH3 mimetic venetoclax was observed in all cell lines tested, with a tendency to stronger synergism at lower venetoclax concentrations. Results indicate that treatment with radioimmunotherapy can sensitize lymphoma to BH3 mimetics.

### Example 6—Combination of Betalutin with MK-1775 and PD-166285

### Objective

Explore the combination of ¹⁷⁷Lu-lilotomab satetraxetan (Betalutin) with the G2/M checkpoint inhibitors MK-1775 and PD-166285 in vitro in different cell lines, animal models and human tumor biopsies of B-cell origin.

### Materials and Methods

### Cell Lines

Ramos (Burkitt's lymphoma, BL), DOHH2 (transformed follicular lymphoma, FL) and Rec-1 (mantle cell lymphoma) cell lines were obtained from ATCC/ECACC and DSMZ. OCI-Ly8 (DLBCL) cell line was obtained from Institute of Oncology Research, Bellinzona, Switzerland. Cells were grown at 37° C. in a humidified atmosphere of 95% air/5% CO₂ in RPMI medium supplemented with 10% heat-inactivated fetal bovine serum, and antibiotics (0.1 U/ml penicillin and 100 μg/ml streptomycin). Mycoplasma contamination was routinely tested using the Mycotest assay from Life technologies (Thermo Fisher Scientific, Waltham, Mass.).

### Antibody Radiolabeling

Lilotomab (Nordic Nanovector, Oslo, Norway) conjugated with p-SCN-benzyl-DOTA (Macrocyclics, Plano, Tx, USA) were labeled with ¹⁷⁷Lu (¹⁷⁷Lu-mAbs) at a specific activity of 200 MBq/mg.

### Animals and Tumor Xenografts

Athymic Nude-Foxn1 mice (athymic mice hereafter) (6-week-old females) from Envigo (Gannat, France) were housed at 22° C. and 55% humidity with a light-dark cycle of 12 h, in pathogen-free conditions and ad libitum supply of food and water. After 1-week acclimatization, 10×10⁶ Ramos or OCI-Ly8 cells were resuspended in 100 μL of fresh serum-free medium before being injected subcutaneously in the flank of athymic mice. All animal experiments were performed in compliance with the French government guidelines and the INSERM standards for experimental animal studies (agreement B34-172-27). They were approved by the Institut de Recherche en Cancérologie de Montpellier (IRCM/INSERM) ethics committee and by the Ethics Committee of the Languedoc Roussillon region (CEEA LR France No. 36) for animal experiments (reference number: 1094).

Treatment of Mice with Tumor Xenografts

Thirteen days post-xenograft, athymic mice bearing 100-200 mm³ Ramos or 8 days post-xenograft, mice bearing 100-200 mm³ OCI-Ly8 cell tumors received one intravenous injection (100 μL) of: i) ¹⁷⁷Lu-lilotomab at 250 MBq or 500 MBq/kg; ii) 2.5 mg/kg rituximab or lilotomab; iii) ¹⁷⁷Lu-lilotomab at 250 MBq+30 mg/kg MK-1775 by gavage (twice a day) from day 1 to 5 post-injection. iv) 30 mg/kg MK-1775 by gavage (twice a day) from day 1 to 5 post-injection. Each treatment group included 6-9 mice.

Tumor growth was evaluated by measuring the tumor volume with a caliper and animal weight was determined twice a week. Mice were sacrificed by CO₂ asphyxiation when the tumor volume reached 2000 mm³, or when the weight loss was higher than 20%, or in the presence of sickness or discomfort.

### Cell Cycle Analysis

Cell cycle was assessed in 1×10⁶ Ramos, DOHH2, Rec-1 and OCI-Ly8 cells grown in 12-well plates and exposed to 0 and 6 MBq/mL of ¹⁷⁷Lu-lilotomab, or to the slightly overestimated corresponding range (0 and 40 μg/mL) of lilotomab or rituximab for 18 h. Cells were harvested at 0 h, 2 h, 18 h, 1 d, 2 d, 3 d, and fixed in 70% ethanol at −20° C. for at least 3 h. After staining with the Muse® Cell Cycle Assay Kit (Merck Millipore, Molsheim, France) using propidium iodide in the dark at room temperature for 30 min, cell cycle distribution was analyzed using a Muse® flow cytometer. The percentage of cells in the G0/G1, S and G2/M was calculated (mean of three experiments in triplicate) and the effect of WEE-1 and MYT-1 kinase inhibitors on the cell cycle was assessed.

Treatment of Human Biopsies with ¹⁷⁷Lu-Lilotomab Alone or in Combination with MK-1775 or PD-166285

Frozen human biopsies from patients with DLBCL or FL were obtained from CHU de Montpellier Plateforme CRB/Hemodiag. Typically, cells were defrosted and grown at 0.5×10⁶ cells/mL in 12-well plates at 37° C. in a humidified atmosphere of 95% air/5% CO₂. Culture medium consisted of RPMI medium supplemented with 20% heat-inactivated fetal bovine serum, antibiotics (0.1 U/ml penicillin and 100 μg/ml streptomycin), 50 ng/mL CD40L (His tagged; R&D system) and 5 μg/mL anti-his-tag antibody (R&D system, Abingdon, UK). After 5 h, they were treated with increasing amount of ¹⁷⁷Lu-lilotomab (0 to 6 MBq/mL) combined or not with MK-1775 or PD-166285 (1 μM) for 18 h. At the end of incubation, 50% of cells were analyzed by flow cytometry and the remaining cells were collected, centrifuged and washed twice with medium before being seeded in 12-well plates for 3 days more. After 3 days, cells were collected and analyzed by flow cytometry. Live/Dead fixable dead cell stain (Fisher scientific), anti-CD45, CD3, CD19, CD20 and CD10 mAbs (BD Pharmigen, Le pont de Claix, France) and anti-Kappa mAb (DAKO, Les Ulis, France) were used and analyzed by flow cytometry to determine quantity and proportion of tumor and non-tumor cells alive.

### Results and Discussion

Inhibitors of G2/M Checkpoint Release Cells from ¹⁷⁷Lu-Lilotomab Induced G2/M Arrest

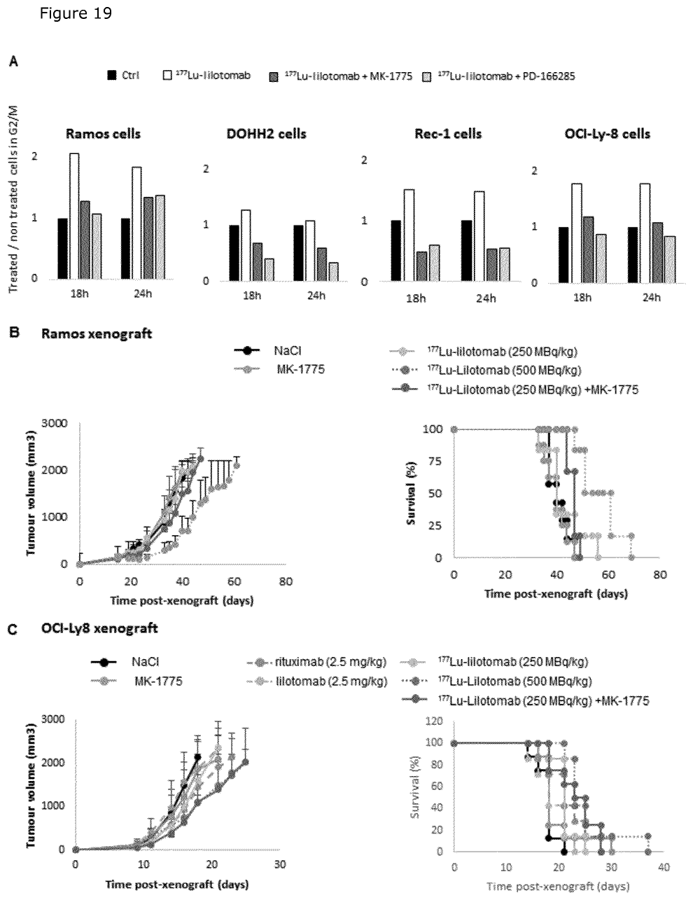

Cells are arrested in G2/M phase of the cell cycle if they are treated with ¹⁷⁷Lu-lilotomab as shown for all cell lines by the increase in treated/non-treated cells in G2/M in FIG. 19 A. Co-incubation with MK-1775 or PD-166285 and ¹⁷⁷Lu-lilotomab leads to a reduction of the fraction of G2/M cells compared with cells exposed to ¹⁷⁷Lu-lilotomab alone in all four cell lines (FIG. 19 A). Our in vitro data indicated that resistance to ¹⁷⁷Lu-lilotomab was mainly associated with arrest of cells in the G2/M phase of the cell cycle upon irradiation. The arrest was strong in Ramos cells and then progressively lower in U2932, Rec-1 cells, OCI-Ly8, and not significant in DOHH2 cells. G2/M cell cycle arrest is likely to be responsible for the strong apoptosis induction observed with ¹⁷⁷Lu-lilotomab, while rituximab-induced apoptosis involves mainly G1 arrest.

During the G2 phase of the cell cycle, CDK1 activity (the master kinase that controls the G2/M transition) becomes activated by A- and B-type cyclins. G2/M cell cycle progression is promoted by CDK1 phosphorylation at Thr161 (located in the activation loop) by the CDK7-containing CAK kinase, a trimetric protein complex consisting of CDK7, cyclin H, and MAT1. Conversely, CDK1 phosphorylation on Tyr15 and Thr14 by WEE-1 and MYT-1, respectively, blocks cells in G2/M. CDK1 Cells can be released from this block by protein phosphatase-mediated dephosphorylation of these residues. These kinases are involved in the DNA-damage response through DNA repair pathways and cell cycle checkpoints that inhibit cell cycle progression during DNA repair. In radiosensitive DOHH2 cells, CDK1 phosphorylation at Tyr15 and Thr14 decreased, whereas phosphorylation at Thr161 increased upon incubation with ¹⁷⁷Lu-lilotomab. Conversely in Ramos, Rec-1, OCI-Ly8, U2932 cells, CDK1 phosphorylation at Tyr15 and Thr14 remained high, whereas phosphorylation at Thr161 was low when measured in Ramos and Rec-1 cells.

### Inhibitors of G2/M Checkpoint Sensitizes Ramos and OCI-Ly8 Tumor Xenograft to ¹⁷⁷Lu-Lilotomab

In Ramos tumor xenograft, combination between 250 MBq/kg ¹⁷⁷Lu-lilotomab and MK-1775 significantly delayed tumor growth compared with 250 MBq/kg ¹⁷⁷Lu-lilotomab alone (p=0.001) (FIG. 19 B). Median survival was significantly increased from 40 days to 47 days (p=0.0156).

In OCI-Ly8 xenografts, lilotomab (nor MK-1775, p=0.625) had no therapeutic efficacy compared with control (p=0.475). When radiolabeled, ¹⁷⁷Lu-lilotomab (250 MBq/kg) significantly improved tumor growth delay (p=0.015) and median survival (p=0.0062). This was enhanced when ¹⁷⁷Lu-lilotomab (250 MBq/kg) was combined with MK-1775 treatment since tumor growth delay was significantly better than with ¹⁷⁷Lu-lilotomab alone (p=0.05). It must be noted that combination was as efficient as 500 MBq/kg ¹⁷⁷Lu-lilotomab alone (p=0.7070) (FIG. 19 C).

Therapeutic Cytotoxicity of ¹⁷⁷Lu-Lilotomab in DLBC and FL Human Biopsies is Improved by Combination with Inhibitors of G2/M Checkpoint

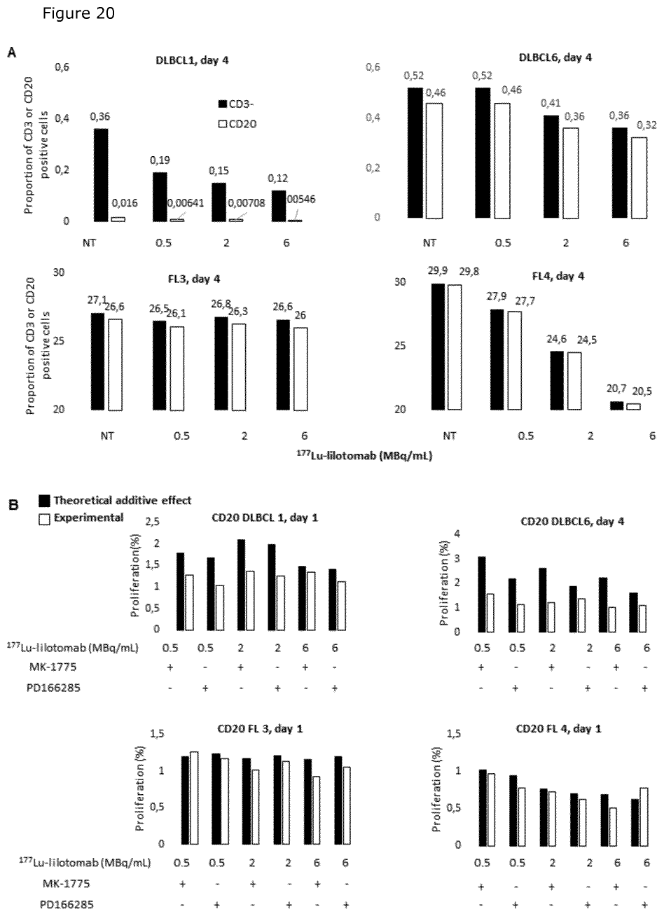

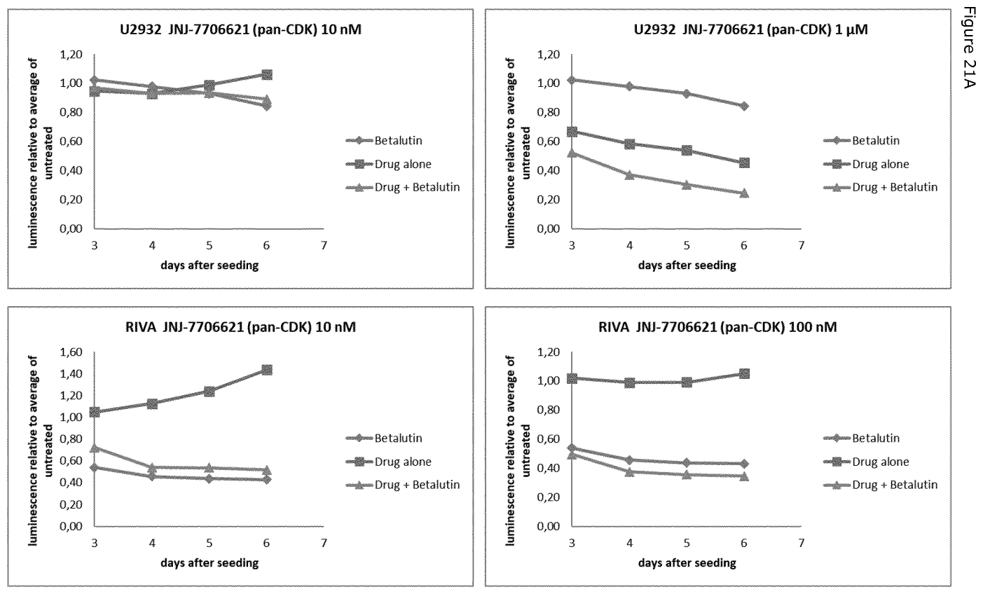

Flow cytometry analysis of cell surface markers (CD3- and CD20) was performed on alive cells isolated from 4 patient biopsies and exposed for 18 h to ¹⁷⁷Lu-lilolotmab or to the combination ¹⁷⁷Lu-lilotomab+MK-1775 or ¹⁷⁷Lu-lilotomab+PD-166285 (FIG. 20 A). We observed that the initial proportion of cells negative to CD3 (CD3-) and positive to CD20 was higher for FL (27.1-29.9%) than for DLBC (0.36-0.52%). ¹⁷⁷Lu-lilotomab reduced the proportion of tumor cells in all the tumors at day 4 (FIG. 20 A).

The effect of G2/M cell cycle arrest inhibitors on ¹⁷⁷Lu-lilotomab cytotoxicity was shown to depend on the ability for the cells to progress through cell cycle. Then, analysis was done either at the end of exposure (day 1) or 3 days later (day 4) (FIG. 20 B). For the 4 biopsies, we calculated what would be theoretical proliferation rate considering additive effect between cytotoxic effects of MK-1775 (or PD-166285) and ¹⁷⁷Lu-lilotomab taken alone. MK-1775 and PD-166285 were shown to increase the cytotoxicity of ¹⁷⁷Lu-lilotomab through synergistic cytotoxic mechanisms (FIG. 20 B).

### Conclusion

We confirmed in vitro and in vivo in Ramos and OCI-Ly8 xenograft models and in 4 human biopsies the role of CDK1 phosphorylation at Tyr15 and Thr14 in G2/M cell cycle arrest and cell death by using WEE-1 and MYT-1 inhibitors. Specifically, WEE-1 inhibition (by MK-1775) sensitized Ramos, Rec-1 and OCI-Ly8 cells to ¹⁷⁷Lu-lilotomab, whereas concomitant WEE-1 and MYT-1 inhibition (by PD-166285) had no additive effect. Similar trend was observed when considering CD3−/CD20+ cells isolated from human biopsies. This suggests that the increased radiosensitivity is mainly determined by WEE-1 activity. For results obtained in biopsies, it must be kept in mind that radiation sensitivity is intimately linked to proliferation index which is a limitation for cells isolated from biopsies.

Combination of ¹⁷⁷Lu-lilotomab with G2/M cell cycle arrest inhibitors would enhance its therapeutic efficacy and may allow to decrease injected amount of radioactivity.

### Example 7—Cell Cycle Kinase Inhibitors Potentiate the Effect of ¹⁷⁷Lu-Lilotomab Satetraxetan in Treatment of Aggressive Diffuse Large B-Cell Lymphoma Cell Lines

### Objective

We reported earlier the differential sensitivity profiles of human diffuse large B-cell lymphoma (DLBCL) cell lines to treatment with ¹⁷⁷Lu-Lilotomab satetraxetan (Betalutin) (see Example 2). U-2932 and RIVA are two aggressive DLBCL cell lines of the activated B-cell like subtype, that show resistance to Betalutin treatment at clinically relevant doses. We have explored if the combination of the radioimmunoconjugate Betalutin, as a vehicle to deliver selectively radiation to tumor cells, and selected inhibitors of mitotic cell cycle kinases can reverse resistance to Betalutin treatment. To this, cells were pre-treated with Betalutin (or not), before removal of excess Betalutin, and seeded onto 384-well plates pre-loaded with selected drugs from a Cancer Compound library (Selleck). Viability was monitored using a luminescence assay (RealTimeGlo). In the screen, we identified drugs that had more than an additive effect in inhibition of proliferation, when combined with Betalutin (examples 2 and 4). In this example, selected candidate hits were tested in extended dose-response experiments for evaluation of synergistic interaction with Betalutin.

### Materials and Methods

### Cell Lines

DLBCL cell lines U2932 and RIVA were maintained in RPMI 1640-GlutaMAX medium (Gibco) supplemented with 15% fetal bovine serum (Biowest) and 1% penicillin-streptomycin (Gibco) at 37° C. in a humidified atmosphere containing 5% CO₂. Cells were split twice a week 1:7 and diluted 2-4 days before start of the experiment, to ensure they are in exponential growth at the beginning of the experiment.

TABLE 9
Cell lines used

| | Lymphoma | Relevant | |
|---|---|---|---|
| Cell line | type | information | medium |
| U2932 | ABC- | BCL2, BCL6, | RPMI 1640, |
| | DLBCL | TP53, RB1 | 100 U/ml |
| | | over-expression, | penicillin/ |
| | | TP53 mutation | streptomycin, |
| | | | 15% FBS |
| RIVA | ABC- | t(4; 8) MYC | RPMI 1640, |
| | DLBCL | rearrangement, | 100 U/ml |
| | | der(18) BCL2 | penicillin/ |
| | | amplification, | streptomycin, |
| | | P15INK4B | 15% FBS |
| | | deletion, | |
| | | P16INK4A | |
| | | deletion, | |
| | | RB1 deletion/ | |
| | | mutation | |

Labeling of Lilotomab with ¹⁷⁷Lu

The chelator p-SCN-Bn-DOTA (satetraxetan, Macrocyclics, TX, USA) was dissolved in 0.005 M HCl, added to the antibody in a 6:1 ratio and pH-adjusted to approximately 8.5 using carbonate buffer. After 45 minutes of incubation at 37° C. the reaction was stopped by the addition of 50 μl per mg of Ab of 0.2 M glycine solution. To remove free satetraxetan the conjugated antibody was washed using Vivaspin 20 centrifuge tubes (Sartorius Stedim Biotech, Gottingen Germany) 4-5 times with NaCl 0.9%. Before labeling with ¹⁷⁷Lu the pH was adjusted to 5.3±0.3 using 0.25 M ammonium acetate buffer. Around 200 MBq of ¹⁷⁷Lu (ITG, Garching, Germany) was added to 0.25 mg of lilotomab-satetraxetan, and incubated for 15 to 30 minutes at 37° C. The radiochemical purity (RCP) of the conjugate was evaluated using instant thin layer chromatography and was higher than 95%. The specific activity was set at 600 MBq/mg (dilution with cold lilotomab-satetraxetan was done as required).

### Immunoreactive Fraction of Betalutin

The immunoreactivity of the radioimmunoconjugates was measured using Ramos cells and a one point modified Lindmo method. The cell concentration used was 75 million cells/ml. The immunoreactivity of the conjugates was higher than 70%.

### Cytotoxicity Studies

### Betalutin Treatment

Cells were treated in 6-well plates without shaking for 18 h with Betalutin at a final concentration of 1 μg/ml for U2932 and 0.5 μg/ml for RIVA. After treatment, PBS was added to the cells, and the cells pelleted. Cells were first resuspended in 1 ml PBS, then washed twice in PBS and finally diluted in growth medium to a final concentration of 2.5*10⁶ cells/ml.

### Real Time Glo Viability Assays.

Cells density measurements were performed the day before seeding. Based on these measurements cells were seeded in 384-well plates at a density of 3000 cells per well in a culturing volume of 25 μl (resulting in start titers: 120 000 cells/ml) using a robot. For measuring viability using Real Time Glo (Promega, WI, USA), the Cell Viability Substrate and NanoLuc® Enzyme were diluted 1:500 in growth medium, and 25 μL of diluted reagents dispensed in each well by a robot. All reagents were equilibrated to 37° C. Cells were incubated with the reaction mix for 1 h at 37° C. before the first measurement of luminescence. Measurements were repeated as often as required within 72 h after adding the reagents. A Tecan SPARK 10M plate reader (Tecan, SUI) was used to measure luminescence at 37° C., with the integration time set to 1 sec.

### Plate Setup

The screen was performed in 384-well plate with selected cell cycle kinase inhibitors (stock solutions 10 mM in DMSO) acquired from SelleckChem (Selleckchem, TX, USA). To include no-drug controls, the drug panel (Table 10) was divided on two plates. Due to previous observations of cells growing poorly in wells located at the edges of the plates, the two outer-most rows and columns were not used. For the primary screen three different drug concentrations were used, 10 nM and 1 μM for U2932 and 10 and 100 nM for RIVA. In validation screen experiments drugs were used at 1, 5, 10, 20, 40, 80, 160, 320, 640, and 1280 nM. For additional combination experiments of JNJ-7706621 and Betalutin, cells were pre-treated with Betalutin as described above, diluted in fresh media and seeded into 384-well plates. JNJ-7706621 was then added to the cells using a Tecan D300e microdispenser (Tecan, SUI).

TABLE 10

| Inhibitor name | reported targets |
|---|---|
| JNJ-7706621 | pan-CDK (CDK1, |
| | CDK2, CDK3, CDK4, |
| | CDK6), AURA, AURB |
| MLN8237 | AURA |
| (Alisertib) | |
| BI2536 | PLK1 |
| GSK461364 | PLK1 |

### Data Analysis

Candidate hits were identified using the Bliss Independence test for drug interaction. The effect of each drug alone at each concentration was calculated as the fraction of dead cells as compared to control cells Fₐ=(1−(RLU_{drug}/avgRLU_{control}), a similar calculation was performed for the effect of Betalutin alone F_{b}=(1−(avgRLU_{Betalutin}/avgRLU_{control}). Through the following equation we found the expected additive effect of the combination of drug+Betalutin: Expected effect (E)F_{ab}=(Fₐ+F_{b}−Fₐ*F_{b}). We subtracted this expected effect from the measured effect (M)F_{ab}=(1−(RLU_{Drug+Betalutin}/avgRLU_{control}) to get a value (Diff)F_{ab} representing how the measured effect differed from the expected additive effect of the combination. This value was normalized to the survival fraction of drug alone: (Diff)F_{ab}#=(Diff)F_{ab}/(1−Fₐ). Furthermore, to get a measure of size of well-to-well variation, we calculated the standard deviation for the effect of Betalutin alone in the 48 control wells on each plate: (STDEV)F_{b}=(RLU_{Betalutin}/RLU_{control})−(averageRLU_{Betalutin}/averageRLU_{control}). Drugs with a value (Diff)F_{ab}#two times higher than the standard deviation of Betalutin treated controls, (STDEV)F_{b}, were scored as hits.

Chou-Talalay model was used for synergy calculations using the Compusyn software. R (goodness of fit to median-effect curve) was calculated for the individual treatments and should be over 0.90 in in vitro culture experiments in order to use the calculated combination index (CI). The CI is an indication of synergy: 0-0.9 is considered synergy. Synergism grading was used as described in WO2006004917A2.

### Results and Discussion

We reported earlier the resistance of two aggressive diffuse large B-cell lymphoma cell lines, U-2932 and RIVA, to Betalutin treatment (Example 2; FIGS. 13-14). Here we used these cell line models to test the effect of cell cycle kinase inhibitors alone or in combination with Betalutin.

U-2932 and RIVA cells were either pre-treated with Betalutin for 18 hrs or not, washed and seeded into microtiter plates pre-printed with cell cycle kinase inhibitors. RealTimeGlo was added at day 3 to monitor proliferation capacity through days 3 to 6. Luminescence read-outs at days 5 and 6 were used for comparative statistical analysis of effect sizes of single and combination treatment. At these time points single treatment with Betalutin inhibited proliferation capacity of U-2932 and RIVA cells by less than 10% and about 50%, respectively.

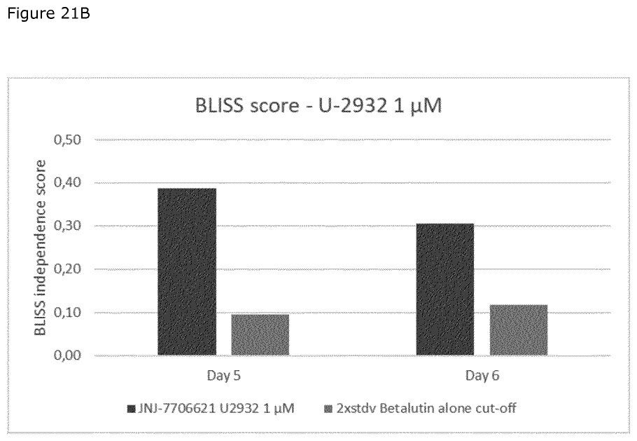

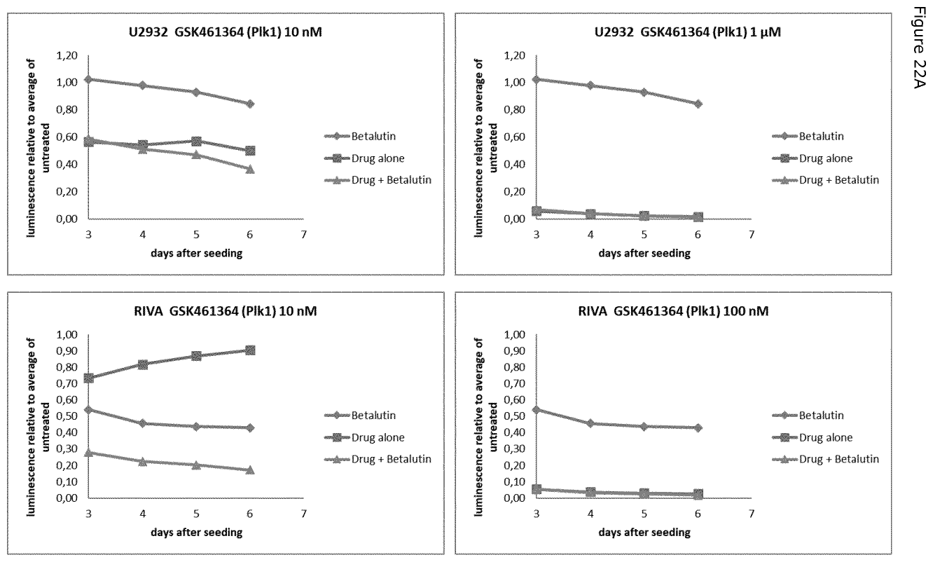

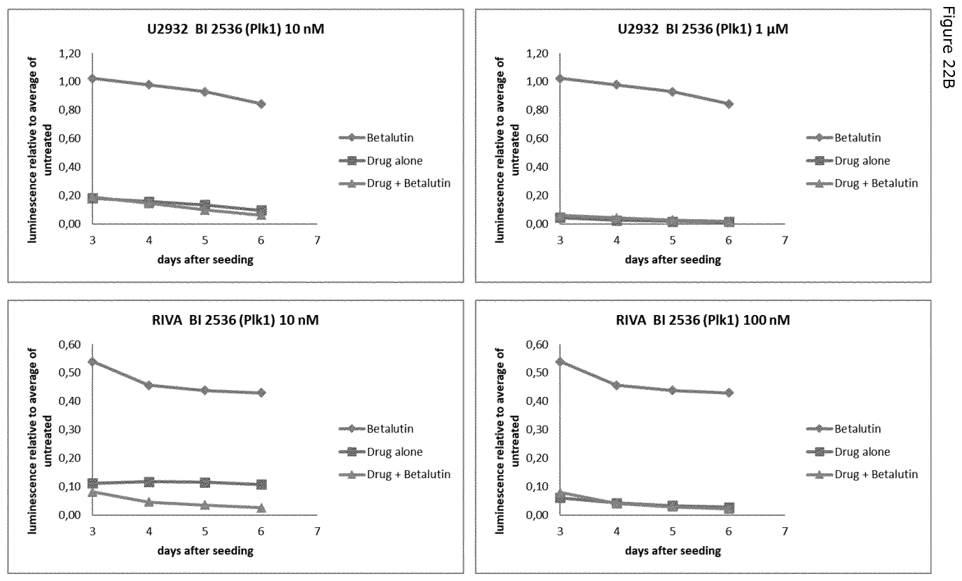

Mono-treatment of U-2932 or RIVA cells with the pan-CDK/AURA/AURB inhibitor JNJ-7706621 had no growth inhibitory effect at 10 and 100 nM dose, respectively (FIG. 21A). Growth of U-2932 cells was inhibited by about 40% five days into treatment with JNJ-7706621 at 1 μM. Betalutin pre-treatment of U-2932 resulted in a combined growth inhibition of more than 60%. BLISS analysis showed that the growth inhibitory effect of the combination was significantly greater than the expected additive effect of the individual drugs (FIG. 3B). Significance is scored as an effect size larger than twice the standard deviation of the effect of Betalutin treatment alone.

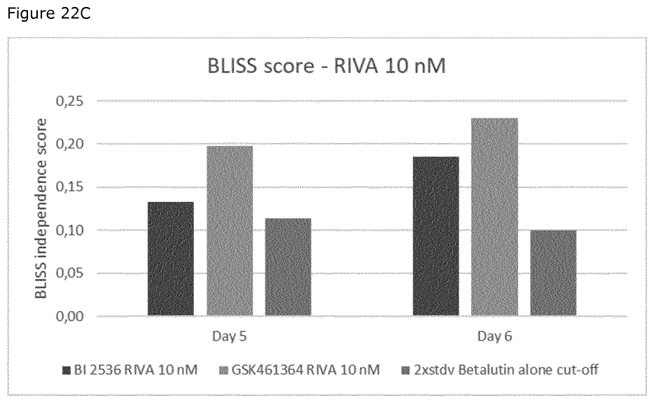

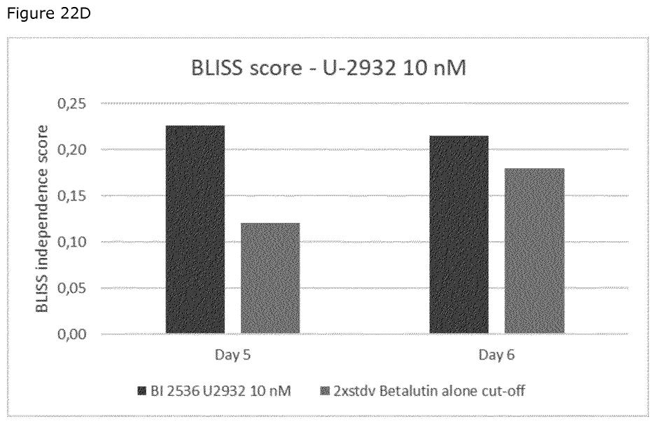

U-2932 or RIVA cells were highly sensitive to inhibition of PLK1 (FIG. 22). Both tested PLK1 inhibitors, GSK461364 and BI2536, blocked proliferation at more than 90% at highest tested concentrations (U-2932: 1 μM; RIVA: 100 nM). In both cell lines GSK461364 (FIG. 22A) showed less potency than BI2536 (FIG. 22B) when tested at 10 nM concentration. In this screening assay BI2536 alone inhibited proliferation by more than 80%. BLISS analysis revealed for both cell lines that the growth inhibitory effect of the combination treatment of Betalutin and GSK461364 was significantly greater than the expected additive effect of the individual single treatments (FIG. 22C). Similar results were found for combination of Betalutin and BI2536 treatment, but significance was only reached in U-2932 cells (FIG. 22D).

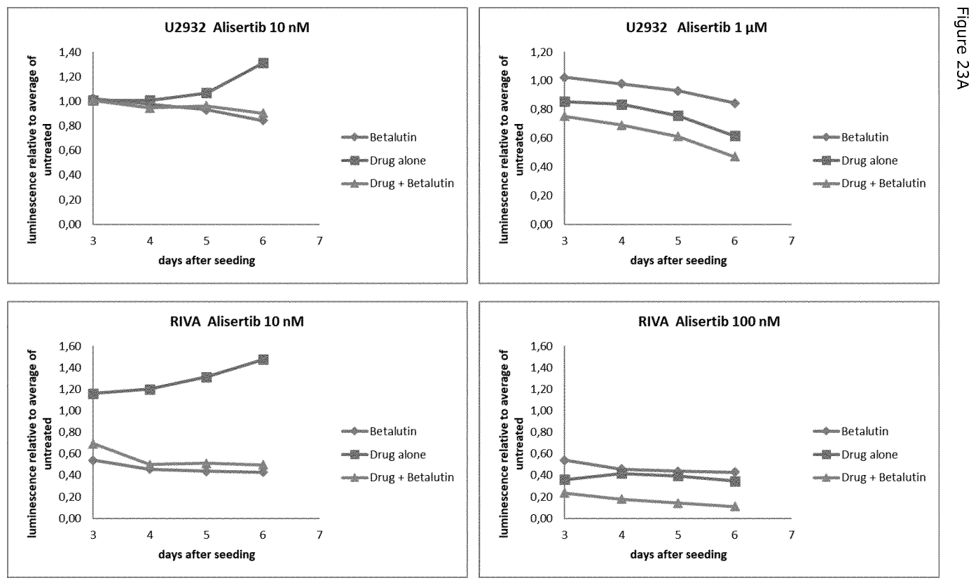

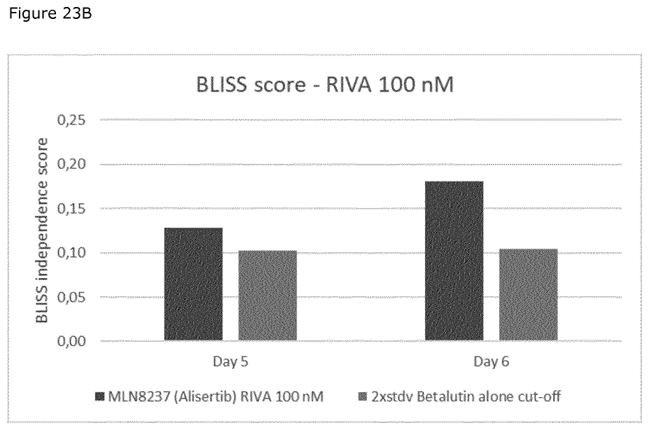

Aurora B kinase inhibitor MLN8237 (Alisertib) was insufficient to inhibit proliferation of U2932 or RIVA cells, when added at 10 nM (FIG. 23A). Growth inhibition was evident in both cell lines at higher drug concentrations, with about 60%) in RIVA cells (day 6, 100 nM) and about 40% in U-2932 cells (day 6, 1 μM). Growth inhibition was enhanced in both cell lines by co-treatment with Betalutin. In RIVA cells the combination of MLN8237 at 100 nM and Betalutin had significantly greater effect than the expected additive effect of the combination (FIG. 23B).

To conclude, a limited screen in two Betalutin treatment resistant U-2932 and RIVA cell identified examples in which the combined treatment of Betalutin with selected cell cycle kinase inhibitors has a larger effect than the expected additive effect of the combination. These examples support the conclusion that cell cycle kinase inhibitors can potentiate the treatment effect of Betalutin.

### Validation Screen

To validate the results of the initial screen, we performed a refined combination treatment screen in the most resistant cell line, U-2932. Here, the combination of three different doses of Betalutin was tested either alone or in combination with 11 different doses of the selected cell cycle kinase inhibitors. Dose-response curves were recorded at four consecutive days and the effects of combination treatments at day 5 tested for synergy using the Chou-Thalaly theorem.

U-2932 cells were pre-treated with Betalutin at 0.5, 1, or 2 μg/ml for 18 hrs and cells washed prior to seeding (in triplicates) into microtiter wells pre-printed with cell cycle kinase inhibitors in a 12-step gradient ranging from 0 to 1280 nM at final concentration. Betalutin-untreated cells were used as control. RealTimeGlo was added at day three and luminescence read daily until day six. Betalutin pre-treatment had a dose dependent growth inhibitory effect, but even at highest dose cells kept about 70%) of the proliferation potential as compared to untreated cells (FIG. 24).

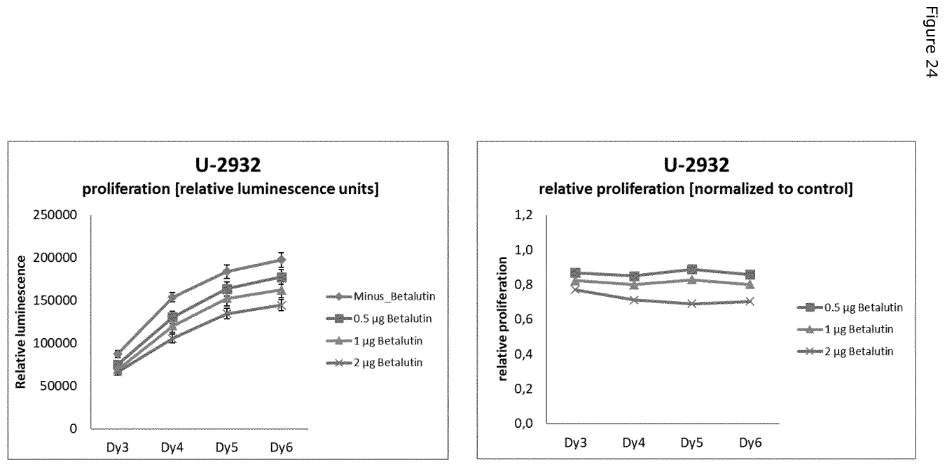

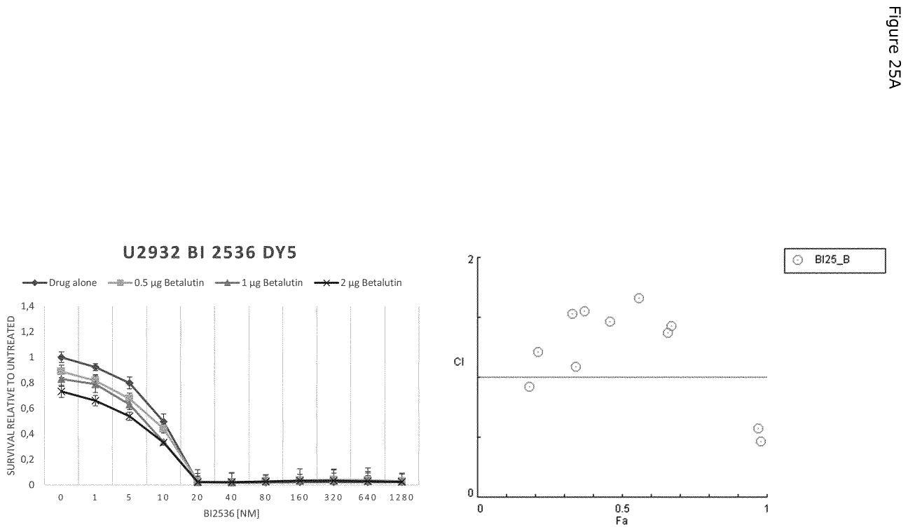

Monotreatment of U-2932 cells with the PLK1 inhibitors BI2536 or GSK461364 blocked proliferation almost completely at concentrations above 40 nM (FIG. 25. Confirming the results of the primary screen, U-2932 cells were more sensitive to BI2536 than GSK461364. CI's were calculated for the combinations of all three Betalutin doses and BI2536 (range 1-20 nM) or GSK461364 (range 1-40 nM), respectively. Synergism of Betalutin and BI2536 was only evident at 40 nM concentration (FIG. 25A; r=0.87)). Synergism of Betalutin and GSK461364 was evident at concentrations of 20 and 40 nM (FIG. 5B; r=0.98).

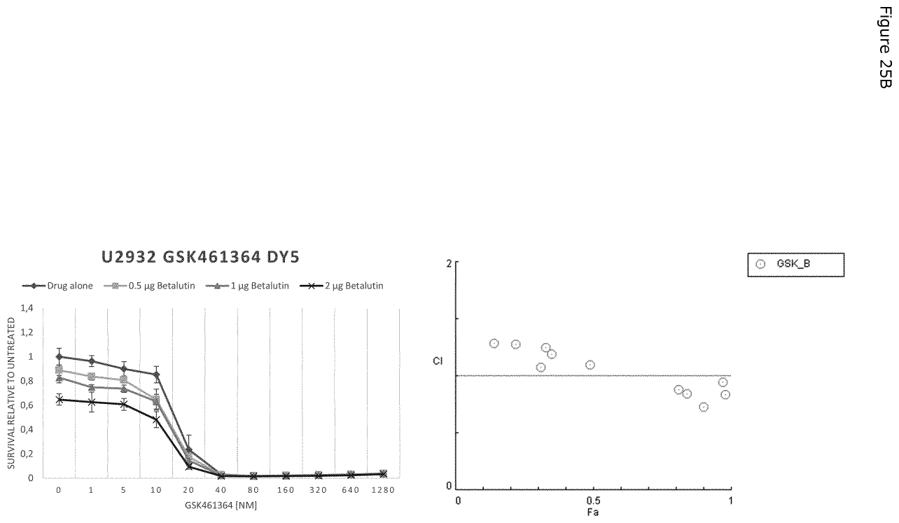

Monotreatment of U-2932 cells with the Aurora B kinase inhibitor MLN8237 (Alisertib) impaired proliferation at concentrations up to 160 nM (FIG. 26). Addition of MLN8237 at concentrations above 160 nM reversed the inhibitory effect, likely due to inhibition of secondary target of anti-proliferative function. Combination indices were calculated for combinations of Betalutin and MLN8237 in the range from 10 to 80 nM. Synergism was evident for all tested Betalutin concentrations in combination with MLN8237 at 20 to 80 nM range (r=1.00).

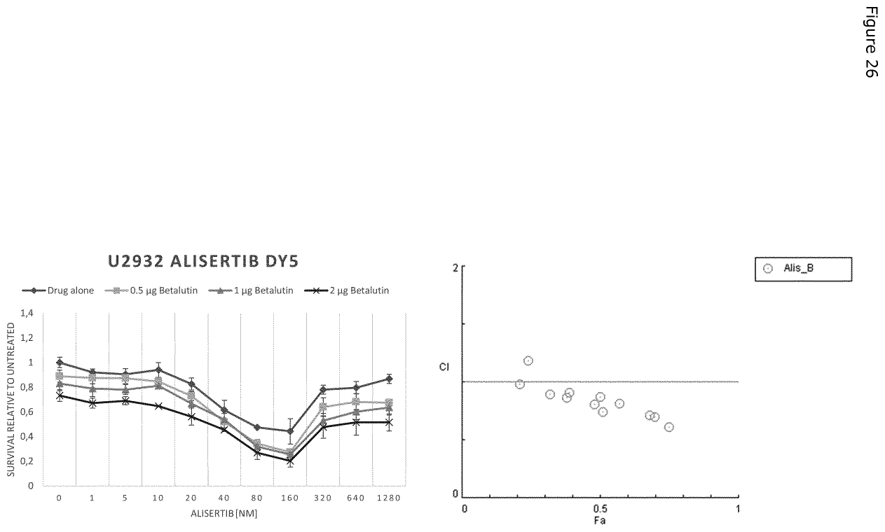

Similar to the results to the primary screen, lower concentrations of JNJ-7706621 did not inhibit proliferation of U-2932 cells (FIG. 27). Growth impairment was not evident at concentrations below 160 nM and reached 30%) at the highest tested dose (1280 nM). In contrast, combination of Betalutin pre-treatment with the pan-CDK inhibitor JNJ-7706621 had a strong growth inhibitory effect at JNJ-7706621 doses above 160 nM, reaching almost 80%) inhibition at 640 nM. The combination index was calculated for JNJ-7706621 doses in the range from 80 to 1280 nM in combination with either 0.5, 1, or 2 μg/ml Betalutin. Synergy was found for all tested combinations (FIG. 27; r=1.00)).

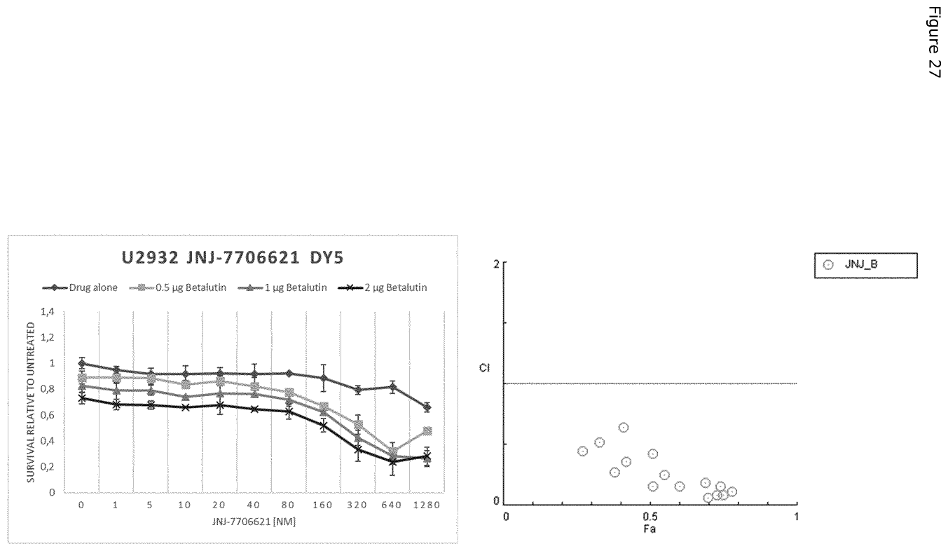

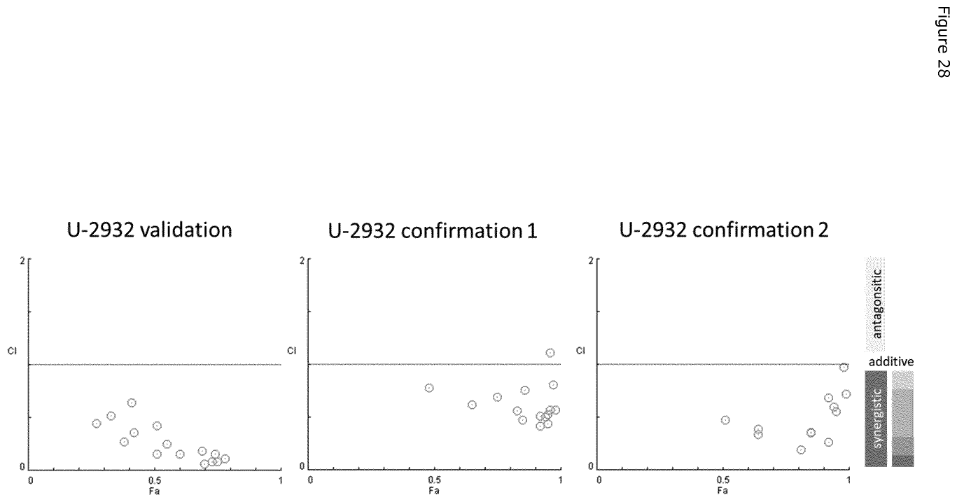

This result was confirmed in two additional, independent, experiments. Here, U-2932 cells were pre-treated with Betalutin at either 0.5, 1, and 2.5 μg/ml (confirmation 1) or 1, 2.5, and 5 μg/ml concentration (confirmation 2). Instead of wash, cells were diluted in fresh medium prior to seeding in triplicates on microtiter wells. JNJ-7706621 was then added using a Tecan D300e dispenser at 100, 266, 707, 1800 and 5000 nM final concentration. Proliferation was assessed using RealTimeGlo as in previous examples and the combination index calculated for all data points read at day 5 (FIG. 28). Synergistic interaction of Betalutin and JNJ-7706621 was observed for all but two combinations (r=0.99 and r=0.96). These exceptions showed additive effect and occurred at highest effect size.

### Conclusions

Comprehensive validation experiments confirm the results of the primary screen. Furthermore, statistical analysis of the collective effect of different combinations of Betalutin and tested cell cycle kinase inhibitors identified drug concentration ranges at which synergism is evident. These results strongly support the claim that addition of cell cycle kinase inhibitors targeting CDKs, PLK1, and/or Aurora kinases can potentiate the treatment effect of Betalutin, reversing resistance in cell line models of aggressive diffuse large B cell lymphoma.

### Example 8: Synergy Between Humalutin and Venetoclax in DLBCL Cell Lines

### Objective

The current study aims to explore if the combination of the anti-CD37 radioimmunoconjugate Humalutin (¹⁷⁷Lu-chHH1.1), as a vehicle to deliver selectively radiation to tumour cells, and the BCL-2 inhibitor venetoclax is synergistic when cell survival is measured 5 days after treatment. Previous studies had shown strong synergy in different Mantle Cell (MC) and Diffuse Large B-Cell Lymphoma (DLBCL) when cell survival was measured 3 days after treatment. The current study is focused on 3 DLBCL cell lines: SUDHL-4, SUDHL-6 and U2932.

### Materials and Methods

### Cell Lines

Cells were grown in RPMI 1640 medium culture media supplemented with Glutamax (Gibco, Paisley, UK), 10-20% heat activated FBS (Gibco) and 1% penicillin-streptomycin (Gibco). The cells were cultured at 37° C. and 5% CO₂.

Cell suspensions were diluted 1:3 to 1:5 with pre-heated medium twice a week and diluted 2-4 days before start of the experiment, to ensure they were in exponential growth at the beginning of the experiment.

TABLE 11
Cell lines used, particulars and culture conditions.

| | Lymphoma | Relevant | |
|---|---|---|---|
| Cell line | type | information | medium |
| SUDHL4 | FL | t(14; 18), | RPMI 1640, |
| | transformed | BH3 | 100 U/ml |
| | to GCB- | insensitive | penicillin/ |
| | DLBCL | | streptomycin, |
| | | | 10% FBS |
| SUDHL6 | GCB- | t(14; 18), | RPMI 1640, |
| | DLBCL | BH3 | 100 U/ml |
| | | sensitive | penicillin/ |
| | | | streptomycin, |
| | | | 20% FBS |
| U2932 | ABC-DLBCL | BCL2, | RPMI 1640, |
| | | BCL6 | 100 U/ml |
| | | over- | penicillin/ |
| | | expression, | streptomycin, |
| | | BH3 | 10% FBS |
| | | sensitive | |

GCB: Germinal Center B-cell
DLBCL: Diffuse Large B-Cell Lymphoma
ABC: Activated B-cell
FL: Follicular Lymphoma

Labelling of chHH1-Satetraxetan with ¹⁷⁷Lu

The chelator p-SCN-Bn-DOTA (satetraxetan, Macrocyclics, TX, USA) was dissolved in 0.005 M HCl, added to the antibody in a 6:1 ratio and pH-adjusted to approximately 8.5 using carbonate buffer. After 45 minutes of incubation at 37° C. the reaction was stopped by the addition of 50 μl per mg of Ab of 0.2 M glycine solution. To remove free satetraxetan the conjugated antibody was washed using Vivaspin 20 centrifuge tubes (Sartorius Stedim Biotech, Gottingen Germany) 4-5 times with NaCl 0.9%. Before labelling with ¹⁷⁷Lu the pH was adjusted to 5.3±0.3 using 0.25 M ammonium acetate buffer. Around 200 MBq of ¹⁷⁷Lu (ITG, Garching, Germany) was added to 0.25 mg of satetraxetan-chHH1, and incubated for 15 to 30 minutes at 37° C. The radiochemical purity (RCP) of the conjugate was evaluated using instant thin layer chromatography and was higher than 95%. The specific activity was set at 600 MBq/mg (dilution with cold chHH1-satetraxetan was done as required).

### Immunoreactive Fraction of Humalutin

The immunoreactivity of the radioimmunoconjugates was measured using Ramos cells and a one point modified Lindmo method. The cell concentration used was 75 million cells/ml. The immunoreactivity of the conjugates was higher than 70%).

### Cytotoxicity Studies

Cells were treated with either Humalutin, venetoclax or a combination of both and seeded into 96 well-plates. At each time point after treatment, cells were incubated with Alamar Blue (Thermo Fisher, DALL1100) for 4 hours and fluorescence measurements were performed using a multiplate reader Fluoroskan ascent FL to assess cell proliferation and viability. All experiments were done in duplicates using 2 samples in each experiment. Data were normalized to the untreated controls. The IC50 was calculated using a log scale transform and non-linear fit with top at 100% and bottom at 0% using Graphpad Prism 8 software (Graphpad Software, San Diego, Calif.).

Treatment with Humalutin

Cells were incubated with 0.25, 0.5, 1, 2.5 or 5 μg/ml of Humalutin in cell culture flasks and incubated at 37° C./5% CO₂. After 18-20 hours cells were washed and resuspended in fresh medium. Cell bound activity after washing was measured using a calibrated gamma detector (Cobra II auto-gamma detector, Packard Instrument Company, Meriden, Conn., USA). Cell concentration and viability were also measured after washing using Guava ViaCount Cell Dispersal Reagent for Flow Cytometry (Merck GaA, Darmstadt, Germany) and measured in a Guava EasyCyte 12HT (Merck KGaA, Darmstadt, Germany) to determine how well the cells survived the incubation. Cells were seeded in 96 well plates. Alamar blue viability measurements were performed 120 h after seeding.

Treatment with Venetoclax

Cells were seeded in 96 well plates pre-coated with 0, 0.5, 1, 2 and 2.5 μM venetoclax in 0.2 ml medium and incubated at 37° C./5% CO₂ for 120 h before the cytotoxic effect was measured using Alamar blue cell viability assay.

Treatment with Humalutin and Venetoclax

Cells were incubated with either 0 (control), 0.5 μg/ml or 1 μg/ml Humalutin for 18 to 20 hours. Cells were then washed and resuspended in fresh medium. Cell bound activity after washing was measured using a calibrated gamma detector (Cobra II auto-gamma detector, Packard Instrument Company, Meriden, Conn., USA). Cell concentration and viability were also measured after washing using Guava ViaCount Cell Dispersal Reagent for Flow Cytometry (Merck GaA, Darmstadt, Germany) and measured in a Guava EasyCyte 12HT (Merck KGaA, Darmstadt, Germany) to determine how well the cells survived the incubation. Cells were then seeded in 96 well plates pre-coated with venetoclax at concentrations between 0.5 and 2.5 μM in 0.2 ml medium and incubated at 37° C./5% CO₂ for 120 hours. Cytotoxic effect was measured using Alamar blue cell viability assay.

### Statistics

The Chou-Talalay model was used for synergy calculations using the Compusyn software. R (goodness of fit) was calculated for the individual treatments and should be over 0.90 in in vitro culture experiments in order to use the calculated combination index (CI) with confidence. The CI is an indication of synergy: 0-0.9 is considered synergy. Synergism grading was used as described in Table 13, WO2006004917A2.

### Results and Discussions

### Humalutin and Venetoclax Alone

The sensitivity to Humalutin and venetoclax was very similar between SUDHL-6 and U2932 while SUDHL-4 was more resistant to both drugs (Table 12, FIG. 1).

TABLE 12
IC50 values for Humalutin and venetoclax
after 120 h incubation time after seeding.

| | IC50 | | IC50 | |
|---|---|---|---|---|
| | Humalutin | R value | venetoclax | R value |
| | at 120 h | Humalutin | at 120 h | venetoclax |
| Cell line | (μg/ml) | at 120 h | (μM) | at 120 h |
| SUDHL-4 | 3.96 | 0.85 | 2.42 | 0.89 |
| SUDHL-6 | 2.63 | 0.98 | 1.16 | 0.96 |
| U2932 | 2.26 | 0.75 | 1.14 | 0.97 |

IC50s values as reported by Graphpad Prism 8; R values as reported by Compusyn software.

### Combination of Humalutin and Venetoclax

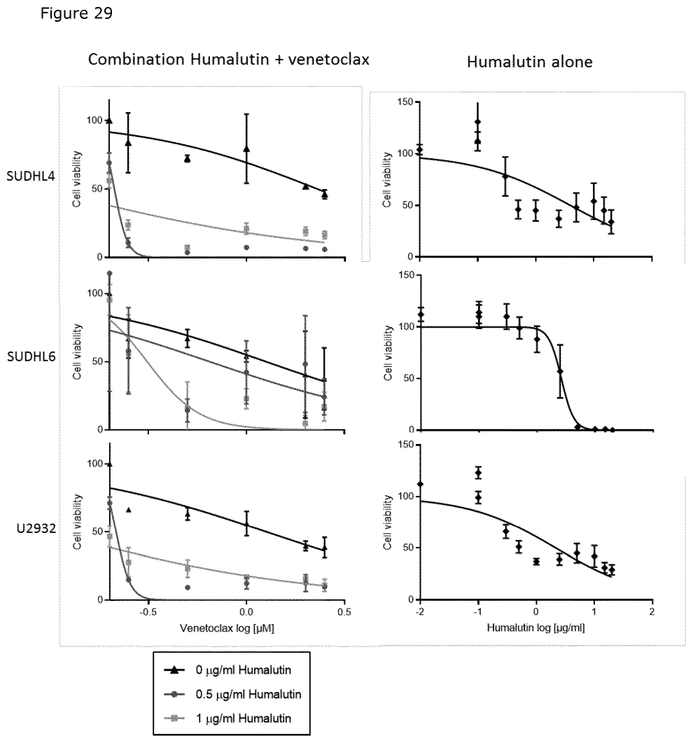

FIG. 29 presents the drug response curves of the combination of both treatment (and of each treatment alone). Calculation of the Combination Index (CI) by the Chou Talalay method showed varying degrees of synergism depending on the cell line and the dose of venetoclax and Humalutin used (Table 13). It is important to notice that the R values for the treatments alone were below 0.9 for the fitting of Humalutin IC50 in the U2932 cell line, which means that the results from the Chou Talalay method should be taken with care.

It is interesting to notice that SUDHL-4 was the most resistant cell line to both Humalutin and venetoclax (in line with being BH3 insensitive) while it showed the stronger synergy.

TABLE 13
Combination Index calculated using the Chou-Talalay method for combination of
0.5 μg/ml or 1 μg/ml Humalutin with different doses of venetoclax.

| | | SUDHL-4 | | SUDHL-6 | | U2932 | |
|---|---|---|---|---|---|---|---|
| Venetoclax | 0.25 | 0.01 | 0.06 | 0.63 | 0.82 | 0.02 | 0.12 |
| | 0.5 | 0.003 | 0.01 | 0.14 | 0.28 | 0.01 | 0.10 |
| | 1 | 0.01 | 0.09 | 0.71 | 0.41 | 0.03 | 0.08 |
| | 2.5 | 0.02 | 0.12 | 0.47 | 0.41 | 0.03 | 0.06 |
| | | 0.5 | 1 | 0.5 | 1 | 0.5 | 1 |

Humalutin μg/ml

| Color code | CI |
|---|---|
| Very strong synergism | <0.1 |
| Strong synergism | 0.1-0.3 |
| Synergism | 0.3-0.7 |
| Moderate/slight synergism | 0.7-0.9 |
| Additive | 0.9-1.1 |

### Conclusions

Synergy between Humalutin and the BCL-2 inhibitor venetoclax was observed in the three DLBCL cell lines tested: SUDHL-4, SUDHL-6 and U2932 5 days after treatment with Humalutin. Results indicate that treatment with radioimmunotherapy can sensitize lymphoma to BCL-2 inhibitors. Further studies in animal models are warranted.

### Example 9: Synergy Between Humalutin and Olaparib in DLBCL Cell Lines

### Objective

The current study aims to explore if the combination of the radioimmunoconjugate Humalutin (¹⁷⁷Lu-chHH1.1), as a vehicle to deliver radiation selectively to tumour cells, and the PARP inhibitor olaparib is synergistic when cell survival is measured 5 days after treatment. Previous studies had shown strong synergy in different Mantle Cell (MCL) and Diffuse Large B-Cell Lymphoma (DLBCL) when cell survival was measured 3 days after treatment. The current study focused on 3 DLBCL cell lines: DOHH2, SUDHL-4 and U2932 and the MCL cell line Granta 519.

### Materials and Methods

### Cell Lines

Cells were grown in RPMI 1640 or DMEM medium culture media supplemented with Glutamax (Gibco, Paisley, UK), 10% heat activated FBS (Gibco) and 1% penicillin-streptomycin (Gibco). The cells were cultured at 37° C. and 5% CO₂.

Cell suspensions were diluted 1:3 to 1:5 with pre-heated medium twice a week and diluted 2-4 days before start of the experiment, to ensure they were in exponential growth at the beginning of the experiment.

TABLE 14
Cell lines used, particulars and culture conditions.

| | Lymphoma | Relevant | |
|---|---|---|---|
| Cell line | type | information | medium |
| Granta | MCL | Partial | DMEM |
| 519 | | ATM | (high glucose), |
| | | loss | 100 U/ml |
| | | | penicillin/ |
| | | | streptomycin, |
| | | | 10% FBS |
| DOHH2 | FL | t(14; 18), | RPMI 1640, |
| | transformed | BH3 | 100 U/ml |
| | to GCB- | sensitive | penicillin/ |
| | DLBCL | | streptomycin, |
| | | | 10% FBS |
| SUDHL4 | FL | t(14; 18), | RPMI 1640, |
| | transformed | BH3 | 100 U/ml |
| | to GCB- | insensitive | penicillin/ |
| | DLBCL | | streptomycin, |
| | | | 10% FBS |
| U2932 | ABC- | BCL2, | RPMI 1640, |
| | DLBCL | BCL6 | 100 U/ml |
| | | over- | penicillin/ |
| | | expression, | streptomycin, |
| | | BH3 | 10% FBS |
| | | sensitive | |

MCL: Mantle Cell Lymphoma
GCB: Germinal Center B-cell
DLBCL: Diffuse Large B-Cell Lymphoma
ABC: Activated B-cell
FL: Follicular Lymphoma

Labelling of chHH1-satetraxetan with ¹⁷⁷Lu

The chelator p-SCN-Bn-DOTA (satetraxetan, Macrocyclics, TX, USA) was dissolved in 0.005 M HCl, added to the antibody in a 6:1 ratio and pH-adjusted to approximately 8.5 using carbonate buffer. After 45 minutes of incubation at 37° C. the reaction was stopped by the addition of 50 μl per mg of Ab of 0.2 M glycine solution. To remove free satetraxetan the conjugated antibody was washed using Vivaspin 20 centrifuge tubes (Sartorius Stedim Biotech, Gottingen Germany) 4-5 times with NaCl 0.9%. Before labelling with ¹⁷⁷Lu the pH was adjusted to 5.3±0.3 using 0.25 M ammonium acetate buffer. Around 200 MBq of ¹⁷⁷Lu (ITG, Garching, Germany) was added to 0.25 mg of satetraxetan-chHH1, and incubated for 15 to 30 minutes at 37° C. The radiochemical purity (RCP) of the conjugate was evaluated using instant thin layer chromatography and was higher than 95%. The specific activity was set at 600 MBq/mg (dilution with cold chHH1-satetraxetan was done as required).

### Immunoreactive Fraction of Humalutin

The immunoreactivity of the radioimmunoconjugates was measured using Ramos cells and a one point modified Lindmo method. The cell concentration used was 75 million cells/ml. The immunoreactivity of the conjugates was higher than 70%.

### Cytotoxicity Studies

Cells were treated with either Humalutin, olaparib or a combination of both and seeded into 96 well-plates. At each time point after treatment, cells were incubated with Alamar Blue (Thermo Fisher, DALL1100) for 4 hours and fluorescence measurements were performed using a multiplate reader Fluoroskan ascent FL to assess cell proliferation and viability. All experiments were done in duplicates using 2 samples in each experiment. Data were normalized to the untreated controls. The IC50 was calculated using a log scale transform and non-linear fit with top at 100% and bottom at 0% using Graphpad Prism 8 software (Graphpad Software, San Diego, Calif.).

Treatment with Humalutin

Cells were incubated with 0.25, 0.5, 1, 2.5 or 5 μg/ml of Humalutin in cell culture flasks and incubated at 37° C./5% CO₂. After 18-20 hours cells were washed and resuspended in fresh medium. Cell bound activity after washing was measured using a calibrated gamma detector (Cobra II auto-gamma detector, Packard Instrument Company, Meriden, Conn., USA). Cell concentration and viability were also measured after washing using Guava ViaCount Cell Dispersal Reagent for Flow Cytometry (Merck GaA, Darmstadt, Germany) and measured in a Guava EasyCyte 12HT (Merck KGaA, Darmstadt, Germany) to determine how well the cells survived the incubation. Cells were seeded in 96 well plates. Alamar blue viability measurements were performed 120 h after seeding.

Treatment with Olaparib

Cells were seeded in 96 well plates pre-coated with concentrations ranging from 1 to 100 μM olaparib in 0.2 ml medium and incubated at 37° C./5% CO₂ for 120 h before the cytotoxic effect was measured using Alamar blue cell viability assay.

Treatment with Humalutin and Olaparib

Cells were incubated with either 0 (control), 0.5 μg/ml or 1 μg/ml Humalutin for 18 to 20 hours. Cells were then washed and resuspended in fresh medium. Cell bound activity after washing was measured using a calibrated gamma detector (Cobra II auto-gamma detector, Packard Instrument Company, Meriden, Conn., USA). Cell concentration and viability were also measured after washing using Guava ViaCount Cell Dispersal Reagent for Flow Cytometry (Merck GaA, Darmstadt, Germany) and measured in a Guava EasyCyte 12HT (Merck KGaA, Darmstadt, Germany) to determine how well the cells survived the incubation. Cells were then seeded in 96 well plates pre-coated with olaparib at concentrations between 1 and 100 μM in 0.2 ml medium and incubated at 37° C./5% CO₂ for 120 hours. Cytotoxic effect was measured using Alamar blue cell viability assay.

### Statistics

The Chou-Talalay model was used for synergy calculations using the Compusyn software. R (goodness of fit) was calculated for the individual treatments and should be over 0.90 in in vitro culture experiments in order to use the calculated combination index (CI) with confidence. The CI is an indication of synergy: 0-0.9 is considered synergy. Synergism grading was used as described in Table 16, WO2006004917A2.

### Results and Discussions

### Humalutin and Olaparib Alone

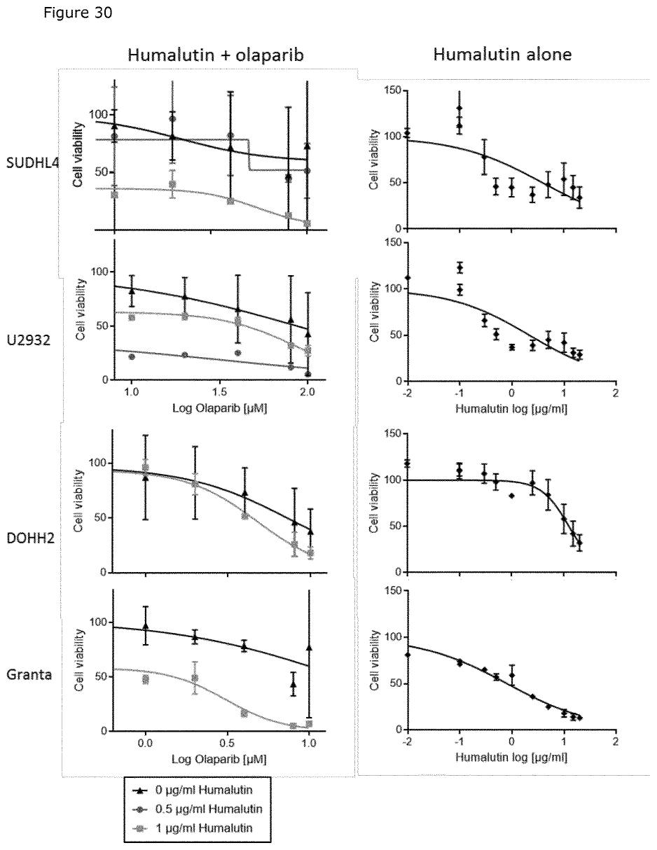

The sensitivity to Humalutin and olaparib varied among the different cell lines (Table 15, FIG. 30). DOHH2 was the most resistant cell line to Humalutin while the most sensitive to olaparib. U2932 was the most sensitive to Humalutin while the most resistant to olaparib.

TABLE 15
IC50 values for Humalutin and olaparib after
120 h incubation time after seeding.

| | IC50 | | IC50 | |
|---|---|---|---|---|
| | Humalutin | R value | olaparib | R value |
| | at 120 h | Humalutin | at 120 h | olaparib |
| Cell line | (μg/ml) | at 120 h | (μM) | at 120 h |
| Granta 519 | 0.81 | 0.96 | 15.04 | 0.87 |
| DOHH2 | 12.5 | 0.87 | 7.56 | 0.98 |
| SUDHL-4 | 3.96 | 0.85 | 22.12 | 0.85 |
| U2932 | 2.26 | 0.75 | 89.08 | 0.99 |

IC50s values as reported by Graphpad Prism 8; R values as reported by Compusyn software.

### Combination of Humalutin and Olaparib

FIG. 1 presents the drug response curves of the combination of both treatments (and of each treatment alone). Calculation of the Combination Index (CI) by the Chou Talalay method showed varying degrees of synergism depending on the cell line and the dose of olaparib and Humalutin used (Table 16). It is important to notice that the R values for the treatments alone were below 0.9 for the fitting of Humalutin IC50 in the U2932 cell line, which means that the results from the Chou Talalay method could be unprecise. Note that for a few combinations of Humalutin and olaparib different levels of antagonism were observe in SUDHL-4, DOHH2 and Granta cell lines. The highest synergy was found for SUDHL-4 treated with 1 μg/ml Humalutin.

TABLE 16
Combination Index calculated using the Chou-Talalay
method for combination of 0.5 μg/ml or 1 μg/ml
Humalutin with different doses of olaparib.

| | | SUDHL-4 | | U2932 | |
|---|---|---|---|---|---|
| Venetoclax | 10 | 2.43 | 0.09 | 0.50 | 0.09 |
| μM | 40 | 3.77 | 0.11 | 0.91 | 0.23 |
| | 80 | 0.50 | 0.06 | 0.54 | 0.12 |
| | 100 | 0.83 | 0.02 | 0.49 | 0.07 |
| | | 0.5 | 1 | 0.5 | 1 |

| | | Humalutin |
|---|---|---|
| | | μg/ml |
| | DOHH2 | Granta |
| 2 | 1.34 | 1.77 |
| 4 | 0.64 | 0.15 |
| 8 | 0.43 | 0.06 |
| 10 | 0.34 | 0.10 |
| | 1 | 1 |

Humalutin
μg/ml

| Colour code | CI |
|---|---|
| Very strong synergism | <0.1 |
| Strong synergism | 0.1-0.3 |
| Synergism | 0.3-0.7 |
| Moderate/slight synergism | 0.7-0.9 |
| Additive | 0.9-1.1 |
| Slight/Moderate antagonism | 1.1-1.45 |
| Antagonism | 1.45-3.3 |
| Strong antagonism | 3.3-10 |

### Conclusions

Synergy between Humalutin and the PARP inhibitor olaparib was observed in all the cell lines tested: SUDHL-4, SUDHL-6, U2932 (DLBCL) and Granta 519 (MCL) 5 days after treatment with Humalutin. Results indicate that treatment with radioimmunotherapy can sensitize lymphoma to PARP inhibitors. Further studies in animal models are warranted.

### Items

1. A composition comprising:
A: a radioimmunoconjugate comprising a monoclonal HH1 antibody and a radionuclide, such as ¹⁷⁷Lu-chHH1.1, ¹⁷⁷Lu-lilotomab, and/or ²¹²Pb-chHH1.1, and
B: an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor.
   2. The composition according to item 1, wherein ¹⁷⁷Lu-chHH1.1, ¹⁷⁷Lu-lilotomab,
   and/or ²¹²Pb-chHH1.1 is linked through a chelating linker.
   3. The composition according to items 1-2, wherein the chelating linker selected from
   the group consisting of p-SCN-benzyl-DOTA, DOTA-NHS-ester, p-SCN-Bn-DTPA, p-SCN-benzyl-TCMC and CHX-A″-DTPA.
   4. The composition according to items 1-3, wherein the chelating linker is
   satetraxetan, also known as p-SCN-benzyl-DOTA.
   5. The composition according to items 1-4, wherein A is ¹⁷⁷Lu-chHH1.1.
   6. The composition according to items 1-5, wherein A is ¹⁷⁷Lu-lilotomab.
   7. The composition according to items 1-6 wherein A is ²¹²Pb-chHH1.1.
   8. The composition according to items 1-7, wherein B is a protein or molecule capable of leading to progression of the cell cycle through the G2/M checkpoint or a protein or molecule capable of inhibiting progression through Mitosis.
   9. The composition according to items 1-8, wherein the protein or molecule leads to lower WEE-1 mediated phosphorylation of cyclin-dependent kinase-1 (CDK1) and progression of the cell cycle through the G2/M checkpoint.
   10. The composition according to items 1-8, wherein the protein or molecule leads to lower MYT-1 mediated phosphorylation of cyclin-dependent kinase-1 (CDK1) and progression of the cell cycle through the G2/M cell cycle arrest.
   11. The composition according to items 1-8, wherein the protein or molecule leads to higher CDK7-containing CAK kinase mediated phosphorylation of cyclin-dependent kinase-1 (CDK1).
   12. The composition according to items 1-8, wherein the protein or molecule is an inhibitor of an AURORA-kinase.
   13. The composition according to items 1-8, wherein the protein or molecule is an inhibitor of protein 14-3-3.
   14. The composition according to items 1-13, wherein the protein or molecule is an inhibitor of proteins involved in G2/M cell cycle arrest, or a protein or molecule capable of inhibiting progression through Mitosis.
   15. The composition according to items 1-14, wherein the protein or molecule is selected from the group consisting of MK-1775, PD-166285, AMG 900, AT7519, AZD7762, CYC116, flavopiridol, GSK461364, JNJ-7706621, LY2603618, NSC 23766, NU6027, PHA-793887, Tosyl-L-Arginine Methyl Ester (TAME), BI6727 (Volasertib), ON-01910 (Rigosertib), HA-1077 (Fasudil), SCH727965 (Dinaciclib), LY2835219, LEE011, Salirasib, K-115 (Ripasudil), PD0332991 (Palbociclib), BI2536, MLN8237, or a 14-3-3 inhibitor, such as difopein.
   16. The composition according to items 1-7, wherein B is a protein or molecule which is a PARP inhibitor.
   17. The composition according to items 1-7 and 16, wherein the PARP inhibitor is selected from the group consisting of olaparib (AZD2281, Ku-0059436), Veliparib (ABT-888), Rucaparib (AG-014699, PF-01367338), Talazoparib (BMN 673), AG-14361, INO-1001 (3-aminobenzamide), A-966492, P334 HCl, Niraparib (MK-4827), UPF 1069, ME0328, NMS-P118, E7449, Picolinamide, benzamide, niraparib (MK-4827) tosylate, NU1025, iniparib (BSI-201), AZD2461, and BGP-15 2HCl.
   18. The composition according to items 1-7, wherein B is a protein or molecule which is a BCL2 inhibitor.
   19. The composition according to items 1-17, wherein the composition is formulated as a pharmaceutical composition.
   20. The composition according to item 19, wherein the pharmaceutical composition comprises one or more pharmaceutically acceptable carriers or adjuvants.
   21. The composition according to items 1-20, for use as a medicament.
   22. The composition for use according to item 21, wherein the medicament is against Non-Hodgkin's lymphoma (NHL).
   23. The composition for use according to item 22, wherein the NHL is selected from the group consisting of transformed follicular lymphoma, diffuse large B-cell lymphoma, mantle cell lymphoma, marginal zone lymphoma, chronic lymphatic leukemia, cutaneous T-cell lymphoma, lymphoplasmacytic lymphoma, marginal zone B-cell lymphoma, MALT lymphoma, small cell lymphocytic lymphoma, Burkitt lymphoma, anaplastic large cell lymphoma, lymphoblastic lymphoma, peripheral T-cell lymphoma, transplant induced lymphoma.
   24. The composition for use according to items 22-23, wherein the use is for a combination therapy where the composition is followed by simultaneous or post-treatment with antibody therapy, immunoconjugate therapy or a combination thereof.
   25. The composition for the use according to anyone of items 22-24, wherein the composition is followed by anti-CD20 antibody therapy in a single administration or in a repeated administration pattern.
   26. The composition for the use according to item 25, wherein the anti-CD20 antibody is rituximab, obinutuzumab or ofatumumab.
   27. A combination of
A: ¹⁷⁷Lu-lilotomab, ¹⁷⁷Lu-chHH1.1, and/or ²¹²Pb-chHH1.1, and
B: an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor,
   for use as a medicament.
   28. The combination of:
   A: ¹⁷⁷Lu-lilotomab, ¹⁷⁷Lu-chHH1.1, and/or ²¹²Pb-chHH1.1, and
   B: an additional drug which can be a protein or molecule capable of leading to progression of the cell cycle through the G2/M checkpoint, a protein or molecule capable of inhibiting progression through Mitosis, a protein or molecule which is a BCL2 inhibitor, or a protein or molecule which is a PARP inhibitor,
   for use according to item 27, wherein the medicament is against Non-Hodgkin's lymphoma.
   29. The combination for use according to item 28, wherein the NHL is transformed follicular lymphoma, diffuse large B-cell lymphoma, mantle cell lymphoma, marginal zone lymphoma, chronic lymphatic leukemia, cutaneous T-cell lymphoma, lymphoplasmacytic lymphoma, marginal zone B-cell lymphoma, MALT lymphoma, small cell lymphocytic lymphoma, Burkitt lymphoma, anaplastic large cell lymphoma, lymphoblastic lymphoma, peripheral T-cell lymphoma, transplant induced lymphoma.
   30. The combination for use according to items 27-29, wherein the use is for a combination therapy where the composition is followed by simultaneous or post-treatment with antibody therapy, immunoconjugate therapy or a combination thereof.
   31. The combination for the use according to anyone of items 27-30, wherein the composition is followed by anti-CD20 antibody therapy in a single administration or in a repeated administration pattern.
   32. The combination for the use according to item 31, wherein the anti-CD20 antibody is rituximab, obinutuzumab, ofatumumab or rituximab biosimilars like Rixathon or Truxima.
   33. The combination for the use according to anyone of items 27-32, wherein ¹⁷⁷Lu-lilotomab, ¹⁷⁷Lu-chHH1.1, and/or ²¹²Pb-chHH1.1 are linked through a chelating linker.
   34. The combination for the use according to anyone of items 26-32, wherein the chelating linker selected from the group consisting of p-SCN-benzyl-DOTA, DOTA-NHS-ester, p-SCN-Bn-DTPA, p-SCN-benzyl-TCMC and CHX-A″-DTPA.
   35. The combination for the use according to anyone of items 27-34, wherein the chelating linker is satetraxetan, also known as p-SCN-benzyl-DOTA.
   36. The combination for the use according to anyone of items 27-35, wherein the protein or molecule leads to lower WEE-1 mediated phosphorylation of cyclin-dependent kinase-1 (CDK1).
   38. The combination for the use according to anyone of items 27-35, wherein the protein or molecule leads to lower MYT-1 mediated phosphorylation of cyclin-dependent kinase-1 (CDK1).
   39. The combination for the use according to anyone of items 27-35, wherein the protein or molecule leads to higher CDK7-containing CAK kinase mediated phosphorylation of cyclin-dependent kinase-1 (CDK1).
   40. The combination for the use according to anyone of items 27-37, wherein the protein or molecule is an inhibitor of G2 cell cycle arrest or inhibitor of M phase progression.
   41. The combination for the use according to anyone of items 27-40, wherein the protein or molecule is selected from the group consisting of MK-1775, PD-166285, AMG 900, AT7519, AZD7762, CYC116, flavopiridol, GSK461364, JNJ-7706621, LY2603618, NSC 23766, NU6027, PHA-793887, Tosyl-L-Arginine Methyl Ester (TAME), BI6727 (Volasertib), ON-01910 (Rigosertib), HA-1077 (Fasudil), SCH727965 (Dinaciclib), LY2835219, LEE011, Salirasib, K-115 (Ripasudil), PD0332991 (Palbociclib), BI2536, MLN8237 (Alisertib), or a protein 14-3-3 inhibitor, such as difopein.
   42. The combination for the use according to anyone of items 27-41, wherein the protein or molecule is MK-1775.
   43. The combination for the use according to anyone of items 27-41, wherein the protein or molecule is PD-166285.
   44. The combination for the use according to anyone of items 27-43, wherein A and B is formulated in one or more pharmaceutical compositions.
   45. The combination for the use according to anyone of items 27-44, wherein the pharmaceutical composition comprises one or more pharmaceutically acceptable carriers or adjuvants.
   46. A composition comprising ¹⁷⁷Lu-lilotomab satetraxetan for use in the treatment of Non-Hodgkin's lymphoma showing reduced inhibitory CDK1 phosphorylation.
   47. The composition according to item 45, wherein the reduced inhibitory CDK1 phosphorylation is from lower WEE-1 mediated phosphorylation of cyclin-dependent kinase-1 (CDK1).
   48. The composition according to item 45, wherein the reduced inhibitory CDK1 phosphorylation is from lower MYT-1 mediated phosphorylation of cyclin-dependent kinase-1 (CDK1).
   49. A composition comprising ¹⁷⁷Lu-lilotomab satetraxetan for use in the treatment of Non-Hodgkin's lymphoma showing higher CDK7-containing CAK kinase mediated phosphorylation of cyclin-dependent kinase-1 (CDK1).
   50. The composition for use according to items 44-50, wherein the NHL is transformed follicular lymphoma, diffuse large B-cell lymphoma, mantle cell lymphoma, marginal zone lymphoma, chronic lymphatic leukemia, cutaneous T-cell lymphoma, lymphoplasmacytic lymphoma, marginal zone B-cell lymphoma, MALT lymphoma, small cell lymphocytic lymphoma, Burkitt lymphoma, anaplastic large cell lymphoma, lymphoblastic lymphoma, peripheral T-cell lymphoma, transplant induced lymphoma.
   51. The composition according to item 14 or the combination according to item 41, wherein the protein or molecule is MK-1775.
   51. The composition according to item 14 or the combination according to item 41, wherein the protein or molecule is PD-166285.
   53. The composition according to item 14 or the combination according to item 41, wherein the protein or molecule is AMG 900.
   54. The composition according to item 14 or the combination according to item 41, wherein the protein or molecule is AZD7762.
   55. The composition according to item 14 or the combination according to item 41, wherein the protein or molecule is JNJ7706621.
   56. The composition according to item 14 or the combination according to item 41, wherein the protein or molecule is CYC116.
   57. The composition according to item 14 or the combination according to item 41, wherein the protein or molecule is AT7519.
   58. The composition according to item 14 or the combination according to item 41, wherein the protein or molecule is LY2603618.
   59. The composition according to item 14 or the combination according to item 41, wherein the protein or molecule is flavopiridol.
   609. The composition according to item 14 or the combination according to item 41, wherein the protein or molecule is GSK461364.
   61. The composition according to item 14 or the combination according to item 41, wherein the protein or molecule is NSC 23766.
   62. The composition according to item 14 or the combination according to item 41, wherein the protein or molecule is NU6027.
   63. The composition according to item 14 or the combination according to item 41, wherein the protein or molecule is PHA-793887.
   64. The composition according to item 14 or the combination according to item 41, wherein the protein or molecule is Tosyl-L-Arginine.
   65. The composition according to item 14 or the combination according to item 41, wherein the protein or molecule is Methyl Ester (TAME).
   66. The composition according to item 14 or the combination according to item 41, wherein the protein or molecule is BI6727 (Volasertib).
   67. The composition according to item 14 or the combination according to item 41, wherein the protein or molecule is ON-01910 (Rigosertib).
   68. The composition according to item 14 or the combination according to item 41, wherein the protein or molecule is HA-1077 (Fasudil).
   69. The composition according to item 14 or the combination according to item 41, wherein the protein or molecule is SCH727965 (Dinaciclib).
   70. The composition according to item 14 or the combination according to item 41, wherein the protein or molecule is LY2835219.
   71. The composition according to item 14 or the combination according to item 41, wherein the protein or molecule is LEE011.
   72. The composition according to item 14 or the combination according to item 41, wherein the protein or molecule is Salirasib.
   73. The composition according to item 14 or the combination according to item 41, wherein the protein or molecule is K-115 (Ripasudil).
   74. The composition according to item 14 or the combination according to item 41, wherein the protein or molecule is PD0332991 (Palbociclib).
   75. The composition according to item 14 or the combination according to item 41, wherein the protein or molecule is a protein 14-3-3 inhibitor.
   76. The composition according to item 14 or the combination according to item 41, wherein the protein or molecule is difopein.
   77. The composition according to item 16 or the combination according to claim for use according to items 27-28, wherein the PARP inhibitor is olaparib (AZD2281, Ku-0059436).
   78. The composition according to item 16 or the combination according to claim for use according to items 27-28, wherein the PARP inhibitor is Veliparib (ABT-888).
   79. The composition according to item 16 or the combination according to claim for use according to items 27-28, wherein the PARP inhibitor is Rucaparib (AG-014699, PF-01367338).
   80. The composition according to item 16 or the combination according to claim for use according to items 27-28, wherein the PARP inhibitor is Talazoparib (BMN 673).
   81. The composition according to item 18 or the combination according to claim for use according to items 27-28, wherein the PARP inhibitor is AG-14361.
   82. The composition according to item 16 or the combination according to claim for use according to items 27-28, wherein the PARP inhibitor is INO-1001 (3-aminobenzamide).
   83. The composition according to item 16 or the combination according to claim for use according to items 27-28, wherein the PARP inhibitor is A-966492.
   84. The composition according to item 16 or the combination according to item for use according to items 27-28, wherein the PARP inhibitor is P334 HCl.
   85. The composition according to item 16 or the combination according to item for use according to items 27-28, wherein the PARP inhibitor is Niraparib (MK-4827).
   86. The composition according to item 16 or the combination according to item for use according to items 27-28, wherein the PARP inhibitor is UPF 1069.
   87. The composition according to item 16 or the combination according to item for use according to items 27-28, wherein the PARP inhibitor is ME0328.
   88. The composition according to item 16 or the combination according to item for use according to items 27-28, wherein the PARP inhibitor is NMS-P118.
   89. The composition according to item 16 or the combination according to item for use according to items 27-28, wherein the PARP inhibitor is E7449.
   90. The composition according to item 16 or the combination according to item for use according to items 27-28, wherein the PARP inhibitor is Picolinamide.
   91. The composition according to item 16 or the combination according to item for use according to items 27-28, wherein the PARP inhibitor is benzamide.
   92. The composition according to item 16 or the combination according to item for use according to items 27-28, wherein the PARP inhibitor is niraparib (MK-4827) tosylate.
   93. The composition according to item 16 or the combination according to item for use according to items 27-28, wherein the PARP inhibitor is NU1025.
   94. The composition according to claim 16 or the combination according to item for use according to items 27-28, wherein the PARP inhibitor is iniparib (BSI-201).
   95. The composition according to item 16 or the combination according to item for use according to claims 27-28, wherein the PARP inhibitor is AZD2461.
   96. The composition according to item 16 or the combination according to item for use according to claims 27-28, wherein the PARP inhibitor is BGP-15 2HCl.
   97. The composition according to item 16 or the combination according to item 41, wherein the protein or molecule is BI2536.
   98. The composition according to item 16 or the combination according to item 41, wherein the protein or molecule is MLN8237 (Alisertib).
   99. The composition according to item 18 or the combination according to item for use according to items 27-28, wherein BCL2 inhibitor is selected from the group consisting of venetoclax (ABT-199, GDC-0199), obatoclax mesylate (GX15-070), HA14(1), ABT-263 (navitoclax), ABT-737, TW-37, AT101, sabutoclax, WEHI-539, A-1155463, gossypolk and AT-101, apogossypol, S1, 2-methoxyantimycin A3, BXI-61, BXI-72, TW37, MIM1, UMI-77, and gambogic acid.
   100. The composition according to item 18 or the combination according to item for use according to claims 27-28, wherein BCL2 inhibitor is venetoclax (ABT-199, GDC-0199).
   101. The composition according to item 18 or the combination according to item for use according to items 27-28, wherein BCL2 inhibitor is obatoclax mesylate (GX15-070).
   102. The composition according to item 18 or the combination according to claim for use according to items 27-28, wherein BCL2 inhibitor is HA14(1).
   103. The composition according to item 18 or the combination according to item for use according to items 27-28, wherein BCL2 inhibitor is ABT-263 (navitoclax).
   104. The composition according to item 18 or the combination according to item for use according to items 27-28, wherein BCL2 inhibitor is TW-37.
   105. The composition according to item 18 or the combination according to item for use according to items 27-28, wherein BCL2 inhibitor is AT101.
   106. The composition according to item 18 or the combination according to item for use according to items 27-28, wherein BCL2 inhibitor is sabutoclax.
   107. The composition according to item 18 or the combination according to item for use according to items 27-28, wherein BCL2 inhibitor is gambogic acid.
   108. The composition according to item 18 or the combination according to item for use according to items 27-28, wherein BCL2 inhibitor is WEHI-539.
   109. The composition according to item 18 or the combination according to item for use according to items 27-28, wherein BCL2 inhibitor is A-1155463.
   110. The composition according to item 18 or the combination according to item for use according to items 27-28, wherein BCL2 inhibitor is gossypol and AT-101.
   111. The composition according to item 18 or the combination according to item for use according to items 27-28, wherein BCL2 inhibitor is apogossypol.
   112. The composition according to item 18 or the combination according to item for use according to items 27-28, wherein BCL2 inhibitor is S1.
   113. The composition according to item 18 or the combination according to item for use according to items 27-28, wherein BCL2 inhibitor is 2-methoxyantimycin A3.
   114. The composition according to item 18 or the combination according to item for use according to items 27-28, wherein BCL2 inhibitor is BXI-61.
   115. The composition according to item 18 or the combination according to item for use according to items 27-28, wherein BCL2 inhibitor is BXI-72.
   116. The composition according to item 18 or the combination according to item for use according to items 27-28, wherein BCL2 inhibitor is TW37.
   117. The composition according to item 18 or the combination according to item for use according to items 27-28, wherein BCL2 inhibitor is MIM1.
   118. The composition according to item 18 or the combination according to item for use according to items 27-28, wherein BCL2 inhibitor is UMI-77.
   119. The composition for use according to claim 23 or 50, or the combination for use according to item 28, wherein the NHL is transformed follicular lymphoma.
   120. The composition for use according to item 23 or 50, or the combination for use according to item 28, wherein the NHL is diffuse large B-cell lymphoma.
   121. The composition for use according to item 23 or 50, or the combination for use according to item 28, wherein the NHL is mantle cell lymphoma.
   122. The composition for use according to item 23 or 50, or the combination for use according to item 28, wherein the NHL is marginal zone lymphoma.
   123. The composition for use according to item 23 or 50, or the combination for use according to item 28, wherein the NHL is chronic lymphatic leukemia.
   124. The composition for use according to item 23 or 50, or the combination for use according to item 28, wherein the NHL is cutaneous T-cell lymphoma.
   125. The composition for use according to item 23 or 50, or the combination for use according to item 28, wherein the NHL is lymphoplasmacytic lymphoma.
   126. The composition for use according to item 23 or 50, or the combination for use according to item 28, wherein the NHL is marginal zone B-cell lymphoma.
   127. The composition for use according to item 23 or 50, or the combination for use according to item 28, wherein the NHL is MALT lymphoma.
   128. The composition for use according to item 23 or 50, or the combination for use according to item 28, wherein the NHL is small cell lymphocytic lymphoma.
   129. The composition for use according to item 23 or 50, or the combination for use according to item 28, wherein the NHL is Burkitt lymphoma.
   130. The composition for use according to item 23 or 50, or the combination for use according to item 28, wherein the NHL is anaplastic large cell lymphoma.
   131. The composition for use according to item 23 or 50, or the combination for use according to item 28, wherein the NHL is lymphoblastic lymphoma.
   132. The composition for use according to item 23 or 50, or the combination for use according to item 28, wherein the NHL is peripheral T-cell lymphoma.
   133. The composition for use according to item 23 or 50, or the combination for use according to item 28, wherein the NHL is transplant induced lymphoma.

1. A method of inhibiting a cancer in a subject comprising administering a combination of:
A: a radioimmunoconjugate comprising a monoclonal HH1 antibody and a radionuclide, and
B: a BCL2 inhibitor to a subject, which has a cancer.

2-20. (canceled)

21. A composition comprising:
A: a radioimmunoconjugate comprising a monoclonal HH1 antibody, and
B: a BCL2 inhibitor.

22. The method according to claim 1, wherein the radioimmunoconjugate comprising a monoclonal HH1 antibody is a chimeric IgG1 HH1 (chHH1.1) or a murine HH1 (HH1, lilotomab).

23. The method according to claim 1, wherein the radionuclide is ¹⁷⁷Lu or ²¹²Pb.

24. The method according to claim 1, wherein the monoclonal HH1 antibody and the radionuclide are linked through a chelating linker.

25. The method according to claim 24, wherein the chelating linker is selected from the group consisting of p-SCN-benzyl-DOTA, DOTA-NHS-ester, p-SCN-Bn-DTPA, p-SCN-benzyl-TCMC and CHX-A″-DTPA.

26. The method of claim 24, wherein the chelating linker is satetraxetan (p-SCN-benzyl-DOTA).

27. The method of claim 1, wherein A is ¹⁷⁷Lu-chHH1.1.

28. The method of claim 1, wherein A is ¹⁷⁷Lu-lilotomab.

29. The method of claim 1, wherein A is ²¹²Pb-chHH1.1.

30. The method of claim 1, wherein the BCL2 inhibitor is selected from the group consisting of venetoclax (ABT-199, GDC-0199), obatoclax mesylate (GX15-070), HA14(1), ABT-263 (navitoclax), ABT-737, TW-37, AT101, sabutoclax, WEHI-539, A-1155463, gossypolk and AT-101, apogossypol, S1, 2-methoxyantimycin A3, BXI-61, BXI-72, TW37, MIM1, UMI-77, and gambogic acid.

31. The method of claim 30, wherein the BCL2 inhibitor is venetoclax (ABT-199, GDC-0199).

32. The composition of claim 21, wherein the composition is formulated as a pharmaceutical composition.

33. The composition of claim 32, wherein the pharmaceutical composition comprises one or more pharmaceutically acceptable carriers or adjuvants.

34. The method of claim 1, wherein the cancer is a Non-Hodgkin's lymphoma (NHL).

35. The method of claim 34, wherein the NHL is selected from the group consisting of transformed follicular lymphoma, diffuse large B-cell lymphoma, mantle cell lymphoma, marginal zone lymphoma, chronic lymphatic leukemia, cutaneous T-cell lymphoma, lymphoplasmacytic lymphoma, marginal zone B-cell lymphoma, MALT lymphoma, small cell lymphocytic lymphoma, Burkitt lymphoma, anaplastic large cell lymphoma, lymphoblastic lymphoma, peripheral T-cell lymphoma, and transplant induced lymphoma.

36. The method of claim 1, further comprising administering an antibody therapy, immunoconjugate therapy or a combination thereof to said subject simultaneously or post-treatment with said combination.

37. The method of claim 36, wherein the said antibody therapy is an anti-CD20 antibody therapy administered in a single administration or in a repeated administration pattern.

38. The method of claim 37, wherein the anti-CD20 antibody is rituximab, obinutuzumab or ofatumumab.
